SEQUENCE LISTING (77 sequences)

SEQ ID NO: 1 - protein, 115 aa, synthetic construct
  REGION 1..115: note = Synthetic: hu1E4 (ver.1)
QVQLVQSGAEVKKPGASVKVSCKASGYTFTNYLIEWVRQAPGQGLEWMGVINPGSGGTNYNEKFKGRVTMTRDTSISTAYMELSRLRSDDTAVYYCASGNYDTYWGQGTLVTVSS

SEQ ID NO: 2 - protein, 115 aa, synthetic construct
  REGION 1..115: note = Synthetic: hu1E4 (ver.2)
QVQLVQSGAEVKKPGASVKVSCKASGYTFTNYLIEWVRQAPGQGLEWIGVINPGSGGTNYNEKFKGKVTLTADKSISTAYMELSRLRSDDTAVYYCASGNYDTYWGQGTLVTVSS

SEQ ID NO: 3 - protein, 115 aa, synthetic construct
  REGION 1..115: note = Synthetic: hu1E4 (ver.3)
QVQLVQSGAEVKKPGASVKVSCKASGYAFTNYLIEWVKQAPGQGLEWIGVINPGSGGTNYNEKFKGKVTLTADKSISTAYMELSRLRSDDTAVYYCASGNYDTYWGQGTLVTVSS

SEQ ID NO: 4 - protein, 115 aa, synthetic construct
  REGION 1..115: note = Synthetic: hu1E4 (ver.4)
QVQLVQSGAEVVKPGASVKVSCKASGYAFTNYLIEWVKQRPGQGLEWIGVINPGSGGTNYNEKFKGKATLTADKSISTAYMELSRLRSDDTAVYYCASGNYDTYWGQGTLVTVSS

SEQ ID NO: 5 - protein, 115 aa, synthetic construct
  REGION 1..115: note = Synthetic: hu1E4 (ver.5)
QVQLVQSGAEVKKPGASVKVSCKASGYAFTNYLIEWVKQAPGQGLEWIGVINPGSGGTNYNEKFKGRATLTRDTSISTAYMELSRLRSDDTAVYFCASGNYDTYWGQGTLVTVSS

SEQ ID NO: 6 - protein, 115 aa, synthetic construct
  REGION 1..115: note = Synthetic: hu1E4 (ver.6)
QVQLVQSGAEVKKPGASVKVSCKASGYAFTNYLIEWVKQAPGQGLEWIGVINPGSGGTNYNEKFKGRATLTADTSISTAYMELSRLRSDDTAVYFCASGNYDTYWGQGTLVTVSS

SEQ ID NO: 7 - protein, 115 aa, synthetic construct
  REGION 1..115: note = Synthetic: hu1E4 (ver.7)
QVQLVQSGAEVKKPGASVKVSCKASGYAFTNYLIEWVKQAPGQGLEWMGVINPGSGGTNYNEKFKGKATLTADKSISTAYMELSRLRSDDTAVYYCASGNYDTYWGQGTLVTVSS

SEQ ID NO: 8 - protein, 115 aa, synthetic construct
  REGION 1..115: note = Synthetic: hu1E4 (ver.8)
QVQLVQSGAEVKKPGASVKVSCKASGYTFTNYLIEWVRQAPGQGLEWMGVINPGSGGTNYNEKFKGRVTMTADKSISTAYMELSRLRSDDTAVYYCASGNYDTYWGQGTLVTVSS

SEQ ID NO: 9 - protein, 115 aa, synthetic construct
  REGION 1..115: note = Synthetic: hu1E4 (ver.9)
QVQLVQSGAEVKKPGASVKVSCKASGYTFTNYLIEWVRQAPGQGLEWIGVINPGSGGTNYNEKFKGRATLTADKSISTAYMELSRLRSDDTAVYYCASGNYDTYWGQGTLVTVSS

SEQ ID NO: 10 - protein, 115 aa, synthetic construct
  REGION 1..115: note = Synthetic: ch1E4 (CPF)_VH
QVQLQQSGAELVRPGTSVKVSCKASGYAFTNYLIEWVKQRPGQGLEWIGVINPGSGGTNYNEKFKGKATLTADKSSSTAYMQLSSLTSDDSAVYFCASGNYDTYWGQGTLVTVSA

SEQ ID NO: 11 - protein, 112 aa, synthetic construct
  REGION 1..112: note = Synthetic: hu1E4 (VL1)
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQSTHVPRTFGQGTKLEIK

SEQ ID NO: 12 - protein, 112 aa, synthetic construct
  REGION 1..112: note = Synthetic: hu1E4 (VL2)
DVVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQSTHVPRTFGQGTKLEIK

SEQ ID NO: 13 - protein, 112 aa, synthetic construct
  REGION 1..112: note = Synthetic: hu1E4 (VL3)
DVVMTQSPLSLPVTLGQPASISCRSSQSLVHSNGNTYLHWFQQRPGQSPRRLIYKVSNRFSGVPDRFSGSGSGTDFTLKISRVEAEDVGVYYCSQSTHVPRTFGGGTKVEIK

SEQ ID NO: 14 - protein, 112 aa, synthetic construct
  REGION 1..112: note = Synthetic: hu1E4 (VL4)
DVVMTQSPLSLPVTLGQPASISCRSSQSLVHSNGNTYLHWYQQRPGQSPRLLIYKVSNRFSGVPDRFSGSGSGTDFTLKISRVEAEDVGVYYCSQSTHVPRTFGGGTKVEIK

SEQ ID NO: 15 - protein, 112 aa, synthetic construct
  REGION 1..112: note = Synthetic: hu1E4 (VL5)
DVVMTQSPLSLPVTLGQPASISCRSSQSLVHSNGNTYLHWYQQRPGQSPRLLIYKVSNRFSGVPDRFSGSGSGTDFTLKISRVEAEDVGVYFCSQSTHVPRTFGGGTKVEIK

SEQ ID NO: 16 - protein, 112 aa, synthetic construct
  REGION 1..112: note = Synthetic: ch1E4 (CPF)_VL
DVVMTQTPLSLPVSLGDQASISCRSSQSLVHSNGNTYLHWYLQKPGQSPKLLIYKVSNRFSGVPDRFSGSGSGTDFTLKISRVEAEDLGVYFCSQSTHVPRTFGGGTKLEIK

SEQ ID NO: 17 - DNA, 345 bp, synthetic construct
  misc_feature 1..345: note = Synthetic: hu1E4 (ver.1)
  source 1..345: mol_type = other DNA
caggtgcaactggtccaatctggtgccgaagtgaaaaagcccggtgcaagtgttaaagtttcctgcaaagcatctggctatacttttactaattatctgatagaatgggtacgacaagctccaggtcaaggtcttgaatggatgggcgtgattaacccagggagtggaggaactaattacaatgagaagttcaaaggacgagtgacaatgacccgtgataccagtattagcaccgcatacatggagttgagcaggcttcgtagcgacgacaccgcagtatattattgtgcttccggcaattatgatacctattgggggcaggggactctcgtaaccgtctccagt

SEQ ID NO: 18 - DNA, 345 bp, synthetic construct
  misc_feature 1..345: note = Synthetic: hu1E4 (ver.2)
  source 1..345: mol_type = other DNA
caagttcagcttgtgcaaagcggagctgaggtcaaaaaaccaggagcatctgtgaaagtgtcatgcaaagcatctggttacacttttactaactatctcattgagtgggtcagacaggctccaggacaagggttggaatggattggggtcatcaaccctgggtcaggggggacaaattacaatgaaaaatttaaagggaaggtgacactcacagcagataagtctatttcaaccgcttatatggaactttcccgccttaggagcgatgatacagcagtctattattgcgcttcagggaactacgacacttactgggggcaaggcacactcgttacagtgtcctcc

SEQ ID NO: 19 - DNA, 345 bp, synthetic construct
  misc_feature 1..345: note = Synthetic: hu1E4 (ver.3)
  source 1..345: mol_type = other DNA
caagtgcagctggtgcagtctggcgcagaagtgaagaaacctggggcttctgtgaaggtcagctgtaaagcttctgggtatgcttttacaaactatctcatagaatgggtcaaacaagctccaggtcagggcctggagtggatcggtgtaatcaaccctggtagtggcggaacaaactacaatgagaagttcaaaggcaaagtgactcttaccgccgacaaaagcatttctacagcttatatggagttgtcaaggttgagaagcgacgacacagcagtttactattgcgcttcaggaaattatgacacatactggggtcagggtacactggtgactgtgtcctct

SEQ ID NO: 20 - DNA, 345 bp, synthetic construct
  misc_feature 1..345: note = Synthetic: hu1E4 (ver.4)
  source 1..345: mol_type = other DNA
caagtccaattggtccagagtggagctgaagttgttaaaccaggagcaagcgttaaagttagttgtaaggcatcaggttacgccttcaccaactacttgattgaatgggtaaagcaacgtcccggtcagggccttgagtggattggggttataaatcctggttctgggggaactaattacaacgagaagttcaagggcaaggccacccttactgccgacaagtccataagcaccgcctatatggaattgagcagattgcgaagtgatgacaccgccgtctactattgtgcttcaggtaattatgatacctattgggggcaaggaacactggtgaccgtatcttcc

SEQ ID NO: 21 - DNA, 345 bp, synthetic construct
  misc_feature 1..345: note = Synthetic: hu1E4 (ver.5)
  source 1..345: mol_type = other DNA
caagtccaactggttcagtctggagctgaagtaaaaaagcctggggccagtgtgaaagtatcctgcaaagccagcggctacgcatttactaattatctgattgaatgggtcaagcaggcaccaggacagggtctggaatggattggggttataaatccaggaagtggggggactaattacaatgagaaatttaaaggccgagcaaccttgacacgcgatacctcaataagtaccgcatatatggaactcagtcgtctgcgaagtgatgataccgctgtttatttctgtgctagtggcaactatgacacatactggggacagggtacattggtaacagtgagtagc

SEQ ID NO: 22 - DNA, 345 bp, synthetic construct
  misc_feature 1..345: note = Synthetic: hu1E4 (ver.6)
  source 1..345: mol_type = other DNA
caagtgcagctggtacagtccggagctgaagtcaagaaaccaggggcctcagtgaaagtttcatgtaaggcttctggatatgcctttaccaactaccttatcgagtgggttaaacaggccccagggcaaggattggagtggatcggggtcatcaatccaggatcaggtggtacaaactataatgagaaattcaagggtagagctactctgacagccgatacaagtatcagcactgcatatatggagttgagccgtctccgcagcgacgacacagccgtatatttctgtgcatctggcaattatgacacatactggggccagggaactcttgtcaccgtctcctca

SEQ ID NO: 23 - DNA, 345 bp, synthetic construct
  misc_feature 1..345: note = Synthetic: hu1E4 (ver.7)
  source 1..345: mol_type = other DNA
caggtccaattggtgcaatcaggggccgaggtcaaaaaacctggagcaagtgttaaagtgagctgtaaggcctctggctacgcctttacaaattatctcatcgaatgggtaaagcaagcaccagggcaaggactcgaatggatgggcgtgattaacccaggatcagggggaaccaattacaacgaaaagtttaaggggaaggctacactgactgctgataaatccatatctactgcatacatggaactgagccggctccgttctgacgacactgctgtctattattgcgcttctggcaattatgatacttattgggggcaaggcactctggttacagtcagctcc

SEQ ID NO: 24 - DNA, 345 bp, synthetic construct
  misc_feature 1..345: note = Synthetic: hu1E4 (ver.8)
  source 1..345: mol_type = other DNA
caagtccagctcgtacaaagtggtgccgaggttaagaaacctggtgctagtgtgaaagtctcatgcaaggctagtggctacacttttaccaattacctgattgaatgggtccgacaagccccaggtcagggattggagtggatgggagtcattaaccctggctcaggtggcactaattataacgaaaagtttaaaggccgtgtgactatgaccgccgacaagagcatctctactgcatacatggaactcagccgtctgcgttctgatgatactgctgtgtattattgtgcatctgggaactacgacacttattggggacagggcactcttgtaaccgtatcctct

SEQ ID NO: 25 - DNA, 345 bp, synthetic construct
  misc_feature 1..345: note = Synthetic: hu1E4 (ver.9)
  source 1..345: mol_type = other DNA
caagtgcagttggtgcaaagtggcgctgaagttaagaagcctggtgcttcagtcaaagtatcttgcaaagctagtggttacacctttaccaactacctgatcgaatgggtcaggcaggcacccggacaaggtttggaatggattggagtcataaaccccggctccggaggaactaactacaacgagaaattcaaaggcagggccactctcaccgctgataaaagtatttcaactgcatatatggaactttctaggcttcggtcagatgacactgctgtctactattgtgcatccggcaattacgatacttattggggccaggggacattggtcacagtgtcttct

SEQ ID NO: 26 - DNA, 345 bp, synthetic construct
  misc_feature 1..345: note = Synthetic: ch1E4 (CPF)_VH
  source 1..345: mol_type = other DNA
caagtccaactccaacaaagtggagcagagttggtgcgcccaggcacaagtgtcaaagttagctgtaaggcttcaggctatgcatttacaaattacttgattgagtgggtgaaacagcgacccggacaaggcttggaatggatcggtgtcatcaatccaggatctgggggaacaaattataacgaaaaattcaaggggaaagccacactcaccgccgataaaagttcatccaccgcatatatgcagcttagctctctcacatctgacgatagcgcagtgtatttctgcgcatccggtaattacgatacttattggggccaaggtactctggtgaccgttagcgcc

SEQ ID NO: 27 - DNA, 336 bp, synthetic construct
  misc_feature 1..336: note = Synthetic: hu1E4 (VL1)
  source 1..336: mol_type = other DNA
gacattgtgatgactcagactccccttagtcttagtgttacccccgggcaaccagcatcaatatcatgccgcagctcacagagtctggttcactctaatggcaacacttatcttcactggtatctccagaaacccggtcaatctccacaactgttgatctacaaagtgtccaatcgcttttcaggcgtgcccgaccgatttagtggtagcgggtcagggaccgactttactctgaaaatttcaagagtagaggctgaagatgtaggagtctactactgctctcaaagtacccatgttcccagaacattcggccagggcaccaaactggagattaag

SEQ ID NO: 28 - DNA, 336 bp, synthetic construct
  misc_feature 1..336: note = Synthetic: hu1E4 (VL2)
  source 1..336: mol_type = other DNA
gatgtagtgatgacacaaacccctttgtcattgagcgtcacccccggacagcccgcttccattagttgccgaagctcacagagtctcgtccactccaacggtaacacatacctccactggtacttgcagaagcctggacagagcccccaattgctgatatataaagtgtcaaacagatttagcggggtgcccgatagattctccggtagcggctcaggcaccgactttacattgaagatcagtcgcgtcgaagcagaggatgttggcgtgtattattgcagtcaaagcacacatgttcctaggacttttgggcagggtactaagttggaaataaag

SEQ ID NO: 29 - DNA, 336 bp, synthetic construct
  misc_feature 1..336: note = Synthetic: hu1E4 (VL3)
  source 1..336: mol_type = other DNA
gatgtagtaatgactcaatctccacttagcctccccgtgacactcggccaaccagcaagcatatcctgtcgttccagccagtccctcgtgcattctaacggcaacacctacttgcattggttccaacagcgtccaggacagagtcctcggcgtttgatatataaggtgtccaaccgattcagcggggttcctgataggttttctggatctggttcaggcaccgatttcactttgaaaatatctcgcgttgaggccgaagacgtaggtgtgtattactgttcccagtccacccatgtgcctcgaaccttcgggggaggtacaaaagtggaaatcaaa

SEQ ID NO: 30 - DNA, 336 bp, synthetic construct
  misc_feature 1..336: note = Synthetic: hu1E4 (VL4)
  source 1..336: mol_type = other DNA
gatgtggtaatgacacagtcccctctctcattgcccgtgacactggggcagcccgcttccattagctgtcgaagtagtcagagcttggttcatagcaatggtaatacatatttgcactggtatcaacaacgtccaggccaatcaccacgactccttatttacaaagtctctaaccggtttagcggtgtaccagaccgtttctctggttcagggagtgggaccgatttcactcttaaaattagtcgggtcgaagccgaagacgttggcgtctactattgcagccagtcaactcatgtaccacgaactttcggcggcggtaccaaggttgagattaag

SEQ ID NO: 31 - DNA, 336 bp, synthetic construct
  misc_feature 1..336: note = Synthetic: hu1E4 (VL5)
  source 1..336: mol_type = other DNA
gatgttgtgatgacacagtccccactctctctgcccgtcacattgggtcaacccgcaagcatctcctgtcgttccagccagagtcttgtacattccaatggcaatacttacctccattggtaccaacagagacccggtcaaagccctcgtctcttgatatataaggtttcaaacaggttctcaggggtacccgaccgattcagcggttcagggtccgggacagacttcaccctcaaaattagtcgtgtggaggctgaggacgtgggtgtatatttctgtagccagagtacccatgtacctcgcacctttgggggaggcaccaaggttgaaattaaa

SEQ ID NO: 32 - DNA, 336 bp, synthetic construct
  misc_feature 1..336: note = Synthetic: ch1E4 (CPF)_VL
  source 1..336: mol_type = other DNA
gacgttgtaatgactcaaacaccactttctttgccagtatcacttggagatcaagcttctatctcatgtaggagctcccagtcactggtgcattcaaacggaaacacataccttcactggtatctgcaaaaaccaggccaatcaccaaagcttttgatatacaaagtgtcaaatcgttttagcggagtaccagaccgattcagcgggagcggaagcgggactgacttcacacttaagattagcagagtggaagccgaagacctgggtgtctatttttgcagccagagcacccatgtcccccgcacattcggaggtggaaccaagctggagataaaa

SEQ ID NO: 33 - protein, 5 aa, synthetic construct
  REGION 1..5: note = Synthetic: HCDR1
NYLIE

SEQ ID NO: 34 - protein, 17 aa, synthetic construct
  REGION 1..17: note = Synthetic: HCDR2
VINPGSGGTNYNEKFKG

SEQ ID NO: 35 - protein, 6 aa, synthetic construct
  REGION 1..6: note = Synthetic: HCDR3
GNYDTY

SEQ ID NO: 36 - protein, 16 aa, synthetic construct
  REGION 1..16: note = Synthetic: LCDR1
RSSQSLVHSNGNTYLH

SEQ ID NO: 37 - protein, 7 aa, synthetic construct
  REGION 1..7: note = Synthetic: LCDR2
KVSNRFS

SEQ ID NO: 38 - protein, 9 aa, synthetic construct
  REGION 1..9: note = Synthetic: LCDR3
SQSTHVPRT

SEQ ID NO: 39 - protein, 330 aa, synthetic construct
  REGION 1..330: note = Synthetic: 1E4 hIgG1 heavy chain constant region (knob)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 40 - protein, 330 aa, synthetic construct
  REGION 1..330: note = Synthetic: 1E4 hIgG1 heavy chain constant region (hole)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 41 - protein, 330 aa, synthetic construct
  REGION 1..330: note = Synthetic: 1E4 hIgG1 heavy chain constant region (knob) (M428L)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVLHEALHNHYTQKSLSLSPGK

SEQ ID NO: 42 - protein, 330 aa, synthetic construct
  REGION 1..330: note = Synthetic: 1E4 hIgG1 heavy chain constant region (hole) (M428L)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVLHEALHNHYTQKSLSLSPGK

SEQ ID NO: 43 - protein, 107 aa, synthetic construct [sequence omitted]

SEQ ID NO: 44 - protein, 15 aa, synthetic construct
  REGION 1..15: note = Synthetic: (G4S)3 Linker - linker between 1E4 hIgG1 and Grabody B scFv
GGGGSGGGGSGGGGS

SEQ ID NO: 45 - protein, 110 aa, synthetic construct
  REGION 1..110: note = Synthetic: Gra-body B anti-IGF1R scFv, VL
QSVLTQPPSASGTPGQRVTISCTGSSSNIGSNDVSWYQQLPGTAPKLLIYANVNRPSGVPDRFSGSKSGTSASLAISGLRSEDEADYYCGTWAGSLNAYVFGCGTKLTVL

SEQ ID NO: 46 - protein, 13 aa, synthetic construct
  REGION 1..13: note = Synthetic: Gra-body B anti-IGF1R scFv, LCDR1
TGSSSNIGSNDVS

SEQ ID NO: 47 - protein, 7 aa, synthetic construct
  REGION 1..7: note = Synthetic: Gra-body B anti-IGF1R scFv, LCDR2
ANVNRPS

SEQ ID NO: 48 - protein, 11 aa, synthetic construct
  REGION 1..11: note = Synthetic: Gra-body B anti-IGF1R scFv, LCDR3
GTWAGSLNAYV

SEQ ID NO: 49 - protein, 20 aa, synthetic construct
  REGION 1..20: note = Synthetic: (G4S)4 Linker between VL and VH
GGGGSGGGGSGGGGSGGGGS

SEQ ID NO: 50 - protein, 121 aa, synthetic construct
  REGION 1..121: note = Synthetic: Gra-body B anti-IGF1R scFv, VH
EVQLLESGGGLVQPGGSLRLSCAASGFTFSSYDMSWVRQAPGKCLEWVSAISYDNANTYYADSVKGRFTISRDNSKNTLYLQMNSLRAEDTAVYYCAKGVLTTLMNWFDYWGQGTLVTVSS

SEQ ID NO: 51 - protein, 10 aa, synthetic construct
  REGION 1..10: note = Synthetic: Gra-body B anti-IGF1R scFv, HCDR1
GFTFSSYDMS

SEQ ID NO: 52 - protein, 17 aa, synthetic construct
  REGION 1..17: note = Synthetic: Gra-body B anti-IGF1R scFv, HCDR2
AISYDNANTYYADSVKG

SEQ ID NO: 53 - protein, 12 aa, synthetic construct
  REGION 1..12: note = Synthetic: Gra-body B anti-IGF1R scFv, HCDR3
GVLTTLMNWFDY

SEQ ID NO: 54 - protein, 251 aa, synthetic construct
  REGION 1..251: note = Synthetic: Gra-body B anti-IGF1R scFv, Full scFv sequence
QSVLTQPPSASGTPGQRVTISCTGSSSNIGSNDVSWYQQLPGTAPKLLIYANVNRPSGVPDRFSGSKSGTSASLAISGLRSEDEADYYCGTWAGSLNAYVFGCGTKLTVLGGGGSGGGGSGGGGSGGGGSEVQLLESGGGLVQPGGSLRLSCAASGFTFSSYDMSWVRQAPGKCLEWVSAISYDNANTYYADSVKGRFTISRDNSKNTLYLQMNSLRAEDTAVYYCAKGVLTTLMNWFDYWGQGTLVTVSS

SEQ ID NO: 55 - protein, 596 aa, synthetic construct
  REGION 1..596: note = Synthetic: hIgG1- Grabody B constant region, incl. Grabody B scFv (hole)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGKGGGGSGGGGSGGGGSQSVLTQPPSASGTPGQRVTISCTGSSSNIGSNDVSWYQQLPGTAPKLLIYANVNRPSGVPDRFSGSKSGTSASLAISGLRSEDEADYYCGTWAGSLNAYVFGCGTKLTVLGGGGSGGGGSGGGGSGGGGSEVQLLESGGGLVQPGGSLRLSCAASGFTFSSYDMSWVRQAPGKCLEWVSAISYDNANTYYADSVKGRFTISRDNSKNTLYLQMNSLRAEDTAVYYCAKGVLTTLMNWFDYWGQGTLVTVSS

SEQ ID NO: 56 - protein, 596 aa, synthetic construct
  REGION 1..596: note = Synthetic: hIgG1 (M428L)-Grabody B constant region, incl. Grabody B scFv (hole)
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVLHEALHNHYTQKSLSLSPGKGGGGSGGGGSGGGGSQSVLTQPPSASGTPGQRVTISCTGSSSNIGSNDVSWYQQLPGTAPKLLIYANVNRPSGVPDRFSGSKSGTSASLAISGLRSEDEADYYCGTWAGSLNAYVFGCGTKLTVLGGGGSGGGGSGGGGSGGGGSEVQLLESGGGLVQPGGSLRLSCAASGFTFSSYDMSWVRQAPGKCLEWVSAISYDNANTYYADSVKGRFTISRDNSKNTLYLQMNSLRAEDTAVYYCAKGVLTTLMNWFDYWGQGTLVTVSS

SEQ ID NO: 57 - protein, 711 aa, synthetic construct
  REGION 1..711: note = Synthetic: hu1E4 IgG1 (V1_VL1) x Grabody B (M428L) heavy chain with Grabody B scFv (hole)
QVQLVQSGAEVKKPGASVKVSCKASGYTFTNYLIEWVRQAPGQGLEWMGVINPGSGGTNYNEKFKGRVTMTRDTSISTAYMELSRLRSDDTAVYYCASGNYDTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVLHEALHNHYTQKSLSLSPGKGGGGSGGGGSGGGGSQSVLTQPPSASGTPGQRVTISCTGSSSNIGSNDVSWYQQLPGTAPKLLIYANVNRPSGVPDRFSGSKSGTSASLAISGLRSEDEADYYCGTWAGSLNAYVFGCGTKLTVLGGGGSGGGGSGGGGSGGGGSEVQLLESGGGLVQPGGSLRLSCAASGFTFSSYDMSWVRQAPGKCLEWVSAISYDNANTYYADSVKGRFTISRDNSKNTLYLQMNSLRAEDTAVYYCAKGVLTTLMNWFDYWGQGTLVTVSS

SEQ ID NO: 58 - protein, 445 aa, synthetic construct
  REGION 1..445: note = Synthetic: hu1E4 IgG1 (V1_VL1) x Grabody B (M428L) heavy chain without Grabody B (knob)
QVQLVQSGAEVKKPGASVKVSCKASGYTFTNYLIEWVRQAPGQGLEWMGVINPGSGGTNYNEKFKGRVTMTRDTSISTAYMELSRLRSDDTAVYYCASGNYDTYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVLHEALHNHYTQKSLSLSPGK

SEQ ID NO: 59 - protein, 219 aa, synthetic construct
  REGION 1..219: note = Synthetic: Light Chain
DIVMTQTPLSLSVTPGQPASISCRSSQSLVHSNGNTYLHWYLQKPGQSPQLLIYKVSNRFSGVPDRFSGSGSGTDFTLKISRVEAEDVGVYYCSQSTHVPRTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 60 - protein, 140 aa, synthetic construct
  REGION 1..140: note = Synthetic: Homo sapiens alpha-synuclein
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 61 - protein, 30 aa, Homo sapiens
  SITE 1..30: note = Human VH1-02 FR1
QVQLVQSGAEVKKPGASVKVSCKASGYTFT

SEQ ID NO: 62 - protein, 14 aa, Homo sapiens
  SITE 1..14: note = Human VH1-02 FR2
WVRQAPGQGLEWMG

SEQ ID NO: 63 - protein, 32 aa, Homo sapiens
  SITE 1..32: note = Human VH1-02 FR3
RVTMTRDTSISTAYMELSRLRSDDTAVYYCAR

SEQ ID NO: 64 - protein, 8 aa, synthetic construct
  REGION 1..8: note = Synthetic: Hu1E4V1 HCDR1 (IMGT)
GYTFTNYL

SEQ ID NO: 65 - protein, 8 aa, synthetic construct
  REGION 1..8: note = Synthetic: Hu1E4V1 HCDR2 (IMGT)
INPGSGGT

SEQ ID NO: 66 - protein, 8 aa, synthetic construct
  REGION 1..8: note = Synthetic: Hu1E4V1 HCDR3 (IMGT)
ASGNYDTY

SEQ ID NO: 67 - protein, 11 aa, synthetic construct
  REGION 1..11: note = Synthetic: Hu1E4VL1 LCDR1 (IMGT)
QSLVHSNGNTY

SEQ ID NO: 68 - skipped

SEQ ID NO: 69 - protein, 9 aa, synthetic construct
  REGION 1..9: note = Synthetic: Hu1E4VL1 LCDR3 (IMGT)
SQSTHVPRT

SEQ ID NO: 70 - protein, 5 aa, synthetic construct
  REGION 1..5: note = Synthetic: Peptide linker
  REPEAT 1..5: note = repeats n times where n is an integer of at least 1
GSGGS

SEQ ID NO: 71 - protein, 5 aa, synthetic construct
  REGION 1..5: note = Synthetic: Peptide linker
  REPEAT 1..5: note = repeats n times where n is an integer of at least 1
GGGGS

SEQ ID NO: 72 - protein, 4 aa, synthetic construct
  REGION 1..4: note = Synthetic: Peptide linker
  REPEAT 1..4: note = repeats n times where n is an integer of at least 1
GGGS

SEQ ID NO: 73 - protein, 7 aa, synthetic construct
  REGION 1..7: note = Synthetic: Peptide linker
AAEPKSS

SEQ ID NO: 74 - protein, 17 aa, synthetic construct
  REGION 1..17: note = Synthetic: Peptide linker
AAEPKSSDKTHTCPPCP

SEQ ID NO: 75 - protein, 4 aa, synthetic construct
  REGION 1..4: note = Synthetic: Peptide linker
GGGG

SEQ ID NO: 76 - protein, 14 aa, synthetic construct
  REGION 1..14: note = Synthetic: Peptide linker
GGGGDKTHTCPPCP

SEQ ID NO: 77 - protein, 1367 aa, Homo sapiens
  SITE 1..1367: note = Human IGF1R
MKSGSGGGSPTSLWGLLFLSAALSLWPTSGEICGPGIDIRNDYQQLKRLENCTVIEGYLHILLISKAEDYRSYRFPKLTVITEYLLLFRVAGLESLGDLFPNLTVIRGWKLFYNYALVIFEMTNLKDIGLYNLRNITRGAIRIEKNADLCYLSTVDWSLILDAVSNNYIVGNKPPKECGDLCPGTMEEKPMCEKTTINNEYNYRCWTTNRCQKMCPSTCGKRACTENNECCHPECLGSCSAPDNDTACVACRHYYYAGVCVPACPPNTYRFEGWRCVDRDFCANILSAESSDSEGFVIHDGECMQECPSGFIRNGSQSMYCIPCEGPCPKVCEEEKKTKTIDSVTSAQMLQGCTIFKGNLLINIRRGNNIASELENFMGLIEVVTGYVKIRHSHALVSLSFLKNLRLILGEEQLEGNYSFYVLDNQNLQQLWDWDHRNLTIKAGKMYFAFNPKLCVSEIYRMEEVTGTKGRQSKGDINTRNNGERASCESDVLHFTSTTTSKNRIIITWHRYRPPDYRDLISFTVYYKEAPFKNVTEYDGQDACGSNSWNMVDVDLPPNKDVEPGILLHGLKPWTQYAVYVKAVTLTMVENDHIRGAKSEILYIRTNASVPSIPLDVLSASNSSSQLIVKWNPPSLPNGNLSYYIVRWQRQPQDGYLYRHNYCSKDKIPIRKYADGTIDIEEVTENPKTEVCGGEKGPCCACPKTEAEKQAEKEEAEYRKVFENFLHNSIFVPRPERKRRDVMQVANTTMSSRSRNTTAADTYNITDPEELETEYPFFESRVDNKERTVISNLRPFTLYRIDIHSCNHEAEKLGCSASNFVFARTMPAEGADDIPGPVTWEPRPENSIFLKWPEPENPNGLILMYEIKYGSQVEDQRECVSRQEYRKYGGAKLNRLNPGNYTARIQATSLSGNGSWTDPVFFYVQAKTGYENFIHLIIALPVAVLLIVGGLVIMLYVFHRKRNNSRLGNGVLYASVNPEYFSAADVYVPDEWEVAREKITMSRELGQGSFGMVYEGVAKGVVKDEPETRVAIKTVNEAASMRERIEFLNEASVMKEFNCHHVVRLLGVVSQGQPTLVIMELMTRGDLKSYLRSLRPEMENNPVLAPPSLSKMIQMAGEIADGMAYLNANKFVHRDLAARNCMVAEDFTVKIGDFGMTRDIYETDYYRKGGKGLLPVRWMSPESLKDGVFTTYSDVWSFGVVLWEIATLAEQPYQGLSNEQVLRFVMEGGLLDKPDNCPDMLFELMRMCWQYNPKMRPSFLEIISSIKEEMEPGFREVSFYYSEENKLPEPEELDLEPENMESVPLDPSASSSSLPLPDRHSGHKAENGPGPGVLVLRASFDERQPYAHMNGGRKNERALPLPQSSTC

(19) United States
(12) Patent Application Publication
(10) Pub. No.: US 2025/0059266 A1
(43) Pub. Date: Feb. 20, 2025

(54) ANTIBODIES FOR TREATING ALPHA-SYNUCLEINOPATHIES

(71) Applicant: ABL BIO INCORPORATED, Seongnam-si (KR)

(72) Inventors: Sungwon AN, Seongnam-si (KR); Dongin KIM, Seongnam-si (KR); Jung-Won SHIN, Seongnam-si (KR); Donghwan KIM, Seongnam-si (KR); Hyesu YUN, Seongnam-si (KR); Jinhyung AHN, Seongnam-si (KR); Byungje SUNG, Seongnam-si (KR); Yong-Gyu SON, Seongnam-si (KR); Jinwon JUNG, Seongnam-si (KR); Bora LEE, Seongnam-si (KR); Daehae SONG, Seongnam-si (KR); Youngdon PAK, Seongnam-si (KR); Kyungjin PARK, Seongnam-si (KR); Juhee KIM, Seongnam-si (KR)

(73) Assignee: ABL BIO INCORPORATED, Seongnam-si (KR)

(21) Appl. No.: 18/938,970

(22) Filed: Nov. 6, 2024

Related U.S. Application Data
(62) Division of application No. 17/743,177, filed on May 12, 2022.

(30) Foreign Application Priority Data
May 12, 2021 (KR) 10-2021-0061407

(51) Int. Cl. C07K 16/18; A61K 39/00; A61P 25/16; C07K 16/28

(52) U.S. Cl. CPC C07K 16/18; A61P 25/16; C07K 16/2863; A61K 2039/505; C07K 2317/24; C07K 2317/31; C07K 2317/35; C07K 2317/565; C07K 2317/567; C07K 2317/622; C07K 2317/92

(57) ABSTRACT

The present disclosure provides isolated binding proteins such as humanized antibodies and antigen-binding fragments thereof that target alpha-synuclein, including multispecific isolated binding proteins that target both alpha-synuclein and insulin-like growth factor 1 receptor. Also provided are methods of using the binding proteins to treat alpha-synucleinopathies.

### CROSS-REFERENCE TO RELATED APPLICATION

This application is a divisional of U.S. patent application Ser. No. 17/743,177, filed May 12, 2022, which claims priority to KR Application No. 10-2021-0061407, filed May 12, 2021. The disclosures of those applications are incorporated by reference herein in their entirety.

### SEQUENCE LISTING

The instant application contains a Sequence Listing that has been submitted electronically in XML format. This Sequence Listing is part of the specification and is hereby incorporated by reference in its entirety. The XML copy, created on Nov. 6, 2024, is named 122548.D1015.xml and is 110,974 bytes in size.

### BACKGROUND

Alpha-synuclein (α-syn) is a neuronal protein involved in vesicle trafficking, synaptic transmission in the brain, and DNA repair. Alpha-synucleinopathies, also called synucleinopathies, are neurodegenerative diseases characterized by the abnormal accumulation of insoluble α-syn in the brain. They include Parkinson's disease, dementia with Lewy bodies, multiple system atrophy, and Alzheimer's disease with amygdala Lewy bodies. Alpha-synucleinopathies are widespread and are a significant cause of human suffering and death. For example, approximately 10 million people worldwide are living with Parkinson's disease. The increasing pathological accumulation of α-syn has been correlated with the progression of disease severity (Henderson et al., Neurosci Lett. (2019) 709:134316). One therapeutic goal in treating α-synucleinopathies is to reduce the abnormal accumulation of α-syn in the brain.

One approach to preventing or reducing the pathological accumulation of α-syn is to develop α-syn-targeting antibodies, typically based on immunoglobulin G (IgG). Although such antibodies have been developed, they have significant clinical limitations (Vaikath et al., J Neurochem. (2019) 150:612-25). One key problem is the inefficiency of therapeutic antibodies to cross the blood brain barrier (BBB), an endothelial cell barrier that limits the passage of molecules in the blood into the brain. Further, previously developed α-syn-targeting antibodies do not differentiate between the physiological, monomeric form of α-syn and the disease-associated oligomeric or protofibril forms of α-syn (Lashuel et al., Nat Rev Neurosci. (2013) 14(1):38-48; Vaikath, supra). In fact, the reduction of the monomeric form of α-syn has been implicated in some forms of neuropathology (Gorbatyuk et al., Mol Ther. (2010) 18(8):1450-7). Thus, there remains a need for improved treatment of α-synucleinopathies.

### SUMMARY

Described herein are recombinant α-syn-binding proteins such as anti-α-syn antibodies and antigen-binding fragments thereof that are useful in treating alpha-synucleinopathies. In one aspect, the present disclosure provides a humanized antibody or antigen-binding fragment thereof that binds to human alpha-synuclein, wherein the antibody or the antigen-binding fragment comprises: a heavy chain variable region (VH) comprising (i) heavy chain complementarity-determining regions (CDR) 1-3 set forth in SEQ ID NOs:33-35, respectively, and (ii) heavy chain framework regions (FRs) 1, 2, and/or 3 derived from a human VH1-02 gene; and a light chain variable region (VL) comprising light chain CDR1-3 set forth in SEQ ID NOs:36-38, respectively. The FR1, FR2, or FR3 derived from the human VH1-02 gene may contain no more than six (e.g., 6, 5, 4, 3, 2, 1, or 0) mutations (e.g., substitutions) relative to the corresponding FR encoded by the human VH1-02 gene. In some embodiments, the antibody or antigen-binding fragment comprises heavy chain FR4 derived from a human JH1, JH4, or JH5 gene.

In one aspect, the present disclosure provides a humanized antibody or antigen-binding fragment thereof that binds to human alpha-synuclein, wherein the antibody or the antigen-binding fragment comprises: a heavy chain variable region (VH) comprising heavy chain complementarity-determining regions (CDR) 1-3 set forth in SEQ ID NOs:64-66, respectively; and/or a light chain variable region (VL) comprising light chain CDR1-3 set forth in SEQ ID NOs:67-69, respectively.

In some embodiments, the antibody or antigen-binding fragment herein has a VH comprising any one of SEQ ID NOs:1 to 9 and a VL comprising any one of SEQ ID NOs:11 to 15. In further embodiments, the VH and the VL comprise: SEQ ID NOs:1 and 11, SEQ ID NOs:2 and 12, SEQ ID NOs:3 and 12, SEQ ID NOs:4 and 12, SEQ ID NOs:7 and 12, SEQ ID NOs:5 and 13, SEQ ID NOs:5 and 15, SEQ ID NOs:6 and 13, SEQ ID NOS:6 and 14, SEQ ID NOs:5 and 14, SEQ ID NOS:8 and 14, or SEQ ID NOs:9 and 14, respectively. In certain embodiments, the VH comprises SEQ ID NO:1, or the VL comprises SEQ ID NO:11. In certain embodiments, the VH comprises SEQ ID NO:1 and the VL comprises SEQ ID NO:11.

In some embodiments, the antibody or antigen-binding fragment herein comprises a human kappa light chain constant region (e.g., SEQ ID NO:43).

In some embodiments, the antigen-binding fragment herein is a single-chain variable fragment (scFv).

In some embodiments, the antibody or antigen-binding fragment is bispecific. The bispecific antibody or antigen-binding fragment may comprise a portion that binds insulin-like growth factor 1 receptor (IGF1R). In some embodiments, the IGF1R-binding portion comprises a VH and a VL, wherein the VH comprises heavy chain CDR1-3 set forth in SEQ ID NOs:51-53, respectively, and the VL comprises light chain CDR1-3 set forth in SEQ ID NOs:46-48, respectively. In further embodiments, the VH and the VL of the IGF1R-binding portion comprise SEQ ID NOs:50 and 45, respectively. In certain embodiments, the IGF1R-binding portion is an scFv (e.g., SEQ ID NO:54).

In some embodiments, the IGF1R-binding portion is fused to the C-terminus of both heavy chains of the antibody.

In some embodiments, the IGF1R-binding portion is fused to the C-terminus of only one heavy chain of the antibody.

In some embodiments, the anti-α-syn antibody of the present disclosure comprises a human IgG1 constant region. This constant region may optionally comprise mutations relative to wildtype human IgG1 sequence. For example, one heavy chain of the antibody may comprise one or more knob mutations (e.g., T366W), while the other heavy chain of the antibody may comprise one or more hole mutations (e.g., T366S, L368A, and Y407V) (all Eu numbering). In further embodiments, the knob heavy chain comprises SEQ ID NO:39 and/or the hole heavy chain comprises SEQ ID NO:40. In some embodiments, the heavy chains of the antibody further comprise an M428L mutation (Eu numbering). Thus, in some embodiments, the knob heavy chain may comprise SEQ ID NO:41 and the hole heavy chain may comprise SEQ ID NO:42.

In some embodiments, the bispecific antibody herein comprises an IGF1R-binding portion fused at the C-terminus of the knob heavy chain. For example, the IGF1R-binding portion is located at the C-terminus of the hole heavy chain, optionally wherein the hole heavy chain comprises SEQ ID NO:55 or 56.

In certain embodiments, the bispecific antibody of the present invention comprises a heavy chain comprising SEQ ID NO:57 and a heavy chain comprising SEQ ID NO:58; and two light chains, each comprising SEQ ID NO:59.

The monospecific or multiple specific (e.g., bispecific) antibodies or antigen-binding fragments of the present disclosure may bind to aggregated or oligomeric alpha-synuclein, optionally with a K_{D} no more than 200 pM, 100 pM, 50 pM, 30 PM, 20 PM or 10 pM, and optionally do not bind specifically to monomeric alpha-synuclein.

In another aspect, the present disclosure provides a pharmaceutical composition comprising a monospecific or multispecific (e.g., bispecific) anti-α-syn antibody or antigen-binding fragment herein and a pharmaceutically acceptable carrier.

In another aspect, the present disclosure provides one or more nucleic acid molecules, such as expression constructs, encoding the present antibody or antigen-binding fragment. In some embodiments, the nucleic acid molecule(s) comprise a nucleotide sequence selected from SEQ ID NOs:17-25 and a nucleotide sequence selected from SEQ ID NOs:27-31. Also provided are host cells (e.g., mammalian host cells) comprising these nucleic acid molecules; and methods of producing the antibody or antigen-binding fragment, by culturing the host cell under conditions that allow expression of the antibody or antigen-binding fragment, and isolating the antibody or antigen-binding fragment from the cell culture.

In another aspect, the present disclosure provides a method of treating an alpha-synucleinopathy (e.g., Parkinson's disease, dementia with Lewy bodies, multiple system atrophy, or Alzheimer's disease with amygdala Lewy bodies) in a human subject in need thereof, comprising administering a therapeutically effective amount of the antibody or antigen-binding fragment herein to the subject. Also provided are antibodies or antigen-binding fragments or pharmaceutical compositions for use in these methods; and use of the antibodies or antigen-binding fragments in the manufacture of a medicament for use in such treatment methods.

### BRIEF DESCRIPTION OF THE FIGURES

FIG. 1 is a graph showing the binding of α-synuclein (α-syn) pre-formed fibrils (PFF) by the indicated monospecific chimeric 1E4 antibody (ch1E4) or monospecific humanized 1E4 antibodies (hu1E4; specifically, hu1E4(V1_VL1), hu1E4(V2_VL2), hu1E4(V3_VL2), hu1E4(V7_VL2), and hu1E4(V4_VL2)) at the indicated antibody concentrations (nM) in ELISA. Mean Value: mean optical density at a wavelength of 450 nm, an indication of the binding.

FIG. 2 is a graph showing the binding of α-syn PFF by monospecific chimeric antibody ch1E4 and monospecific hu1E4 antibodies (hu1E4(V8_VL4) and hu1E4(V9_VL4)) in ELISA. “hIgG1”: An unrelated human IgG1 as a negative control.

FIG. 3 is a graph showing the binding of α-syn PFF by monospecific ch1E4 and bispecific hu1E4 antibodies based on Grabody B (an anti-IGF1R scFv) at the indicated antibody concentrations in ELISA. The bispecific antibodies (BsAbs) were hu1E4(V1_VL1)×Grabody B; hu1E4(V2_VL2)×Grabody B; hu1E4(V3_VL2)×Grabody B; hu1E4(V4_VL2)×Grabody B; and hu1E4(V7_VL2)×Grabody B.

FIG. 4 is a graph showing the binding of α-syn PFF by ch1E4 and bispecific hu1E4 antibodies based on Grabody B at the indicated antibody concentrations in ELISA. The BsAbs were hu1E4(V5_VL3)×Grabody B; hu1E4(V5_VL5)×Grabody B; hu1E4(V6_VL3)×Grabody B antibody; hu1E4(V6_VL4)×Grabody B; and hu1E4(V5_VL4)×Grabody B.

FIG. 5 is a graph showing the binding of α-syn PFF by recombinant mouse 1E4, ch1E4, and hu1E4(V1_VL1) at the indicated concentrations in ELISA.

FIG. 6 is a graph showing the binding of α-syn PFF by BsAb hu11F11 (ver.2)×Grabody and BsAb hu1E4(V1_VL1)×Grabody B at the indicated antibody concentrations in ELISA.

FIG. 7 is a graph showing the binding of oligomeric α-syn by the present antibodies and previously known antibodies in ELISA. The antibodies were hu11F11(ver.2); chimeric 9E4 antibody (ch9E4, aka prasinezumab; Roche/Prothena); hu1E4(V1_VL1); hu1E4(V1_VL1)×Grabody B; NI-202 (Biogen); and mouse hybridoma 1E4 (hy1E4).

FIG. 8 is a graph showing the binding of oligomeric α-syn by BsAbs hu11F11(ver.2)×Grabody B and hu1E4(V1_VL1)×Grabody B in ELISA.

FIG. 9 is a set of plots showing the binding of α-syn PFF by BsAbs hu11F11(ver.2)×Grabody B and hu1E4(V1_VL1)×Grabody B as measured by surface plasmon resonance (SPR). Each plot shows the resonance units (RU) across time (seconds). The data were used to calculate the quantitative properties of α-syn PFF/antibody binding, such as the association rate constant (kₐ), dissociation rate constant (k_{d}), and equilibrium dissociation constant (K_{D}).

FIG. 10 is a set of plots showing the binding of α-syn PFF by hu1E4(V1_VL1)×Grabody B and commercially available (monospecific) anti-α-syn antibodies (BA149 (BioArctic), ch9E4, and NI-202) as measured by SPR.

FIG. 11 is a set of plots showing the binding of monomeric α-syn by hu1E4(V1_VL1)×Grabody B, ch9E4, and hIgG1 (negative control) as measured by SPR.

FIG. 12 is a graph showing the phagocytosis of extracellular α-syn PFF by BV-2 cells (microglial cells) in the presence of ch1E4, ch9E4, or hIgG1 (negative control). “gMFI”: geometric mean fluorescence intensity, as measured using fluorescence activated cell sorting (FACS) analysis.

FIG. 13 is a graph showing the phagocytosis of extracellular α-syn PFF by THP-1 cells (monocytes) in the presence of ch1E4×Grabody B or hIgG1 (negative control).

FIG. 14 is a histogram showing the phagocytosis of extracellular α-syn PFF by BV2 cells in the presence of hu1E4(V1_VL1)×Grabody B, hu1E4(V7_VL2)×Grabody B antibody, hu1E4(V5_VL5)×Grabody B, or hIgG1 at different concentrations.

FIG. 15 is a graph showing the phagocytosis of extracellular α-syn PFF by BV2 cells in the presence of ch1E4 (monospecific), hu1E4(V1_VL1)×Grabody B, or hIgG1 at different concentrations.

FIG. 16 is a graph showing the phagocytosis of extracellular α-syn PFF by BV2 cells in the presence of hu1E4(V1_VL1)×Grabody B, ch9E4, or hIgG1 at different concentrations.

FIG. 17 is a graph showing the phagocytosis of extracellular α-syn PFF by BV2 cells in the presence of hu1E4(V1_VL1)×Grabody B, hu11F11(ver.2)×Grabody B, or hIgG1 at different concentrations.

FIG. 18 is a set of images showing the immunohistochemistry of α-syn in mouse brain sections containing the cerebral cortex, striatum, and substantia nigra (SN), including the substantia nigra pars compacta (SNpc) and substantia nigra pars reticularis (SNpr). The brain sections came from mThy-1 mice, which overexpress human α-syn, and were stained with ch1E4, hu11F11, NI-202, ch9E4, or BA149 antibody.

FIG. 19 is a set of images showing the immunohistochemistry of α-syn in mouse brain sections containing the amygdala, the Cornu Ammonis (CA3) of the hippocampus, and the dentate gyrus (DG) of the hippocampus, including the granular cell layer of the dentate gyrus (GrDG) and the polymorph layer of the dentate gyrus (PoDG). The brain sections were stained with ch1E4, hu11F11, NI-202, ch9E4, and BA149. Brain tissue came from mThy-1 mice, which overexpress human α-syn.

FIG. 20 is a set of images showing the immunohistochemistry of phosphorylated α-syn in postmortem brain tissue from a human patient diagnosed with Parkinson's disease. The phosphorylated α-syn binding abilities of ch1E4 and the commercially available Syn303 antibody (BioLegend) were compared in adjacent sections.

FIG. 21 is a set of images showing the distribution of ch1E4 and ch1E4×Grabody B in the cerebral cortex, CA3 of the hippocampus, and substantia nigra of mThy-1 mice. The amounts of anti-α-syn antibodies (as quantified by the levels of human IgG1, as both mono- and bi-specific ch1E4 contain a human IgG1 constant region) are shown in bar graphs on the right.

FIG. 22 is a set of images showing the immunohistochemistry of phosphorylated α-syn in mThy-1 mice following in vivo administration of ch1E4 or ch1E4×Grabody B. The images show the cerebral cortex, amygdala, and dentate gyrus of the hippocampus. Quantifications of the phosphorylated α-syn are shown in bar graphs on the right.

FIG. 23 is a set of images and bar graphs showing the immunohistochemistry of phosphorylated α-syn in mThy-1 mice following in vivo administration of hu11F11×Grabody B, hu1E4(V1_VL1)×Grabody B, and negative control IgG (IgG). Wildtype (WT) mice did not overexpress human α-syn.

FIG. 24 is a set of graph and images showing the amelioration of α-syn propagation in dopaminergic neurons derived from induced pluripotent stem cells (iPSCs) from Parkinson's disease patients. The cells were treated with monospecific hu1E4(V1_VL1) and hu11F11 antibodies at doses of 1 μg/ml, 3 μg/ml, and 5 μg/ml. The cells were labeled using DAPI (blue) and dopaminergic neurons were localized by staining for tyrosine hydroxylase (TH; green); α-syn is shown in red.

### DETAILED DESCRIPTION

The present disclosure provides isolated binding proteins, such as antibodies and antigen-binding fragments thereof, that bind α-synuclein. These binding proteins comprise humanized antibody heavy chain variable regions (VH) and light chain variable regions (VL) containing specific antigen-binding sequences.

These binding proteins such as antibodies and antigen-binding fragments are advantageous as therapeutics for alpha-synucleinopathies because they bind preferentially to disease-associated aggregated, oligomeric, and/or phosphorylated forms of α-syn, as opposed to monomeric α-syn. α-syn monomers exist in large amounts in the brain and blood of a healthy person, and they play an important role in regulating neurotransmitter release. But only a trace amount of the disease-associated α-syn aggregates is typically found in the brains of alpha-synucleinopathy patients. When an anti-α-syn antibody binds well to both α-syn monomers and aggregates, a significant amount of the antibody would bind to the monomers in the body, because the monomers are present in a much larger amount. As a result, the antibody would have little effect on removing the pathogenic aggregates. Further, if a therapeutic anti-α-syn antibody binds to the monomer form to a significant extent, the normal physiological functions of α-syn may be negatively affected. Therefore, the preferential binding of the present α-syn-binding proteins for the disease-associated forms is important in treating α-syn-related diseases.

Further, the present inventors have surprisingly discovered that the present binding proteins such as humanized antibodies bind to the disease-associated forms of α-syn with higher affinity than the parental mouse antibody from which they are derived. These humanized antibodies also are expected to have low immunogenicity in human patients because no or very few back mutations were required during the humanization process to maintain the antigen-binding affinity of the engineered antibodies.

The present disclosure also provides multispecific (e.g., bispecific) binding proteins such as antibodies that bind to both α-syn and IGF1R, where the IGF1R-binding portions of the binding proteins significantly improve the proteins' ability to cross the brain-blood barrier, improving the patient's exposure to the binding proteins at disease sites.

The present binding proteins are superior to previously known anti-α-syn antibodies in demonstrating high-affinity binding to aggregated and oligomeric α-syn. This superiority is preserved in the anti-α-syn/IGF1R bispecific format.

Unless otherwise indicated, α-synuclein herein refers to human α-synuclein. An human α-synuclein polypeptide sequence is available under UniProt Accession No. Q6QBS3 (SEQ ID NO:60). Unless otherwise indicated, IGF1R herein refers to human IGF1R. A human IGF1R polypeptide sequence is available under UniProt Accession No. P08069 (SEQ ID NO:77).

### I. α-Syn-Binding Proteins

The α-syn-binding proteins herein include chimeric or humanized anti-α-syn antibodies with murine-originated antigen-binding domains. The term “antibody” herein includes monospecific and multispecific (e.g., bispecific) antibodies. The term “antibody” (Ab) or “immunoglobulin” (Ig), as used herein, may refer to a tetramer comprising two heavy (H) chains and two light (L) chains interconnected by disulfide bonds. Each heavy chain is comprised of a heavy chain variable region or domain (VH) and a heavy chain constant region (CH). Each light chain is composed of a light chain variable region or domain (VL) and a light chain constant region (CL). The VH and VL domains can be subdivided further into regions of hypervariability, termed “complementarity-determining regions” (CDRs), interspersed with regions that are more conserved, termed “framework regions” (FRs). Each VH and VL is composed of three CDRs (HCDR herein designates a CDR from the heavy chain; and LCDR herein designates a CDR from the light chain) and four FRs, arranged from amino-terminus to carboxyl-terminus in the following order: FR1, CDR1, FR2, CDR2, FR3, CDR3, and FR4.

The precise amino acid sequence boundaries of a given CDR or FR can be defined by several well-known systems, including those described by Kabat et al., 5th Ed., Public Health Service, National Institutes of Health, Bethesda, MD (1991) (“Kabat” system); Al-Lazikani et al., J Mol Biol. (1997) 273:927-48) (“Chothia” system); MacCallum et al., J Mol Biol. (1996) 262:732-45 (“contact” system); Lefranc et al., Dev Comp Immunol. (2003) 27(1):55-77 (“IMGT” system); Honegger and Plückthun, J Mol Biol. (2001) 309(3):657-70 (“Aho” system); and Whiteleggand Rees, Protein Eng. (2000) 13(12):819-24 (“AbM” system). The boundaries of a given CDR or FR may vary depending on the system used. For example, the Kabat system is based on sequence alignments, while the Chothia system is based on structural information. Numbering for both the Kabat and Chothia systems is based upon the most common antibody region sequence lengths, with insertions accommodated by insertion letters, for example, “30a.” The two systems place certain insertions and deletions (“indels”) at different positions, resulting in differential numbering. The contact system is based on analysis of complex crystal structures and is similar in many respects to the Chothia system. In certain embodiments, the CDRs of the antibodies described herein can be defined by a system selected from Kabat, Chothia, IMGT, Aho, AbM, or combinations thereof.

The antibodies provided herein may be of any immunoglobulin isotype, such as IgG (e.g., IgG1, IgG2, IgG3, or IgG4). The antibodies herein preferably comprise a human IgG (e.g., IgG1) constant region. In some embodiments, the IgG constant region may comprise mutations that improve the therapeutic potential of the antibody, such as mutations that reduce or eliminate effector functions of the antibody (see, e.g., Wang et al., Protein Cell (2018) 9(1):63-73). For example, the monospecific or multispecific antibody herein may comprise a human IgG1 constant region with the mutation L235E, “LALA” mutations (L234A/L235A), or “LALAGA” mutations (L234A/L235A/G237A) (Eu numbering). The IgG constant region may comprise mutations that improve the serum half-life of the antibody, such as the M428L mutation (Eu numbering). The IgG constant region may comprise mutations that improve manufacturing and yield of the antibody; see, e.g., description below regarding knob-in-hole mutations for bispecific antibodies. Such mutated human constant regions are still considered “human” constant regions herein.

In preferred embodiments, the binding proteins of the present disclosure are humanized antibodies, e.g., humanized IgG1 or IgG4 antibodies. A “humanized” antibody is an antibody in which all or substantially all CDR amino acid residues are derived from non-human CDRs (e.g., mouse) and all or substantially all FR amino acid residues are derived from human FRs (i.e., acceptor). A humanized antibody may also include at least a portion of an antibody heavy and/or light chain constant region derived from a human antibody. Compared to the non-human parental antibody from which a humanized antibody is derived, a humanized antibody has reduced immunogenicity to humans. To retain the specificity and affinity of the parental antibody, some FR residues in the human acceptor may be substituted with corresponding residues from the non-human parental antibody (back mutations).

In some embodiments, the binding proteins herein are antigen-binding fragments of full (tetrameric) antibodies. The term “antigen-binding fragment” or “antigen-binding portion” herein encompasses genetically engineered and/or otherwise modified forms of immunoglobulins that do not have the conventional full-length tetrameric structure. The term encompasses intrabodies, peptibodies, diabodies, triabodies, tetrabodies, Fv, Fab, Fab′, Fab′-SH, F(ab′)₂, single-chain antibody molecules (e.g., scFv or sFv), tandem di-scFv, and tandem tri-scFv.

The α-syn-binding proteins of the present disclosure are derived from mouse monoclonal antibody 1E4. Humanized versions of the mouse 1E4 parental antibody are termed hu1E4 antibodies herein. In some embodiments, the binding proteins such as hu1E4 antibodies or antigen-binding fragments thereof comprise one or more (e.g., two or three) of the HCDRs of one of the following humanized VH sequences. These sequences are aligned below with the human germline gene VH1-02 used as the acceptor for humanization. Kabat-defined HCDRs are italicized; mutations from the human germline sequence are bolded and underlined.

| | |
|---|---|
| | FR1 CDR1 |
| Human VH1-02 | QVQLVQSGAEVKKPGASVKVSCKASGYTFT----- |
| VHhu1E4_ver. 1 | QVQLVQSGAEVKKPGASVKVSCKASGYTFTNYLIE |
| VHhu1E4_ver. 2 | QVQLVQSGAEVKKPGASVKVSCKASGYTFTNYLIE |
| VHhu1E4_ver. 3 | QVQLVQSGAEVKKPGASVKVSCKASGYAFTNYLIE |
| VHhu1E4_ver. 4 | QVQLVQSGAEVVKPGASVKVSCKASGYAFTNYLIE |
| VHhu1E4_ver. 5 | QVQLVQSGAEVKKPGASVKVSCKASGYAFTNYLIE |
| VHhu1E4_ver. 6 | QVQLVQSGAEVKKPGASVKVSCKASGYAFTNYLIE |
| VHhu1E4_ver. 7 | QVQLVQSGAEVKKPGASVKVSCKASGYAFTNYLIE |
| VHhu1E4_ver. 8 | QVQLVQSGAEVKKPGASVKVSCKASGYTFTNYLIE |
| VHhu1E4_ver. 9 | QVQLVQSGAEVKKPGASVKVSCKASGYTFTNYLIE |
| | FR2 CDR2 |
| Human VH1-02 | WVRQAPGQGLEWMG----------------- |
| VHhu1E4_ver.1 | WVRQAPGQGLEWMGVINPGSGGTNYNEKFKG |
| VHhu1E4_ver.2 | WVRQAPGQGLEWIGVINPGSGGTNYNEKFKG |
| VHhu1E4_ver.3 | WVKQAPGQGLEWIGVINPGSGGTNYNEKFKG |
| VHhu1E4_ver.4 | WVKQRPGQGLEWIGVINPGSGGTNYNEKFKG |
| VHhu1E4_ver.5 | WVKQAPGQGLEWIGVINPGSGGTNYNEKFKG |
| VHhu1E4_ver.6 | WVKQAPGQGLEWIGVINPGSGGTNYNEKFKG |
| VHhu1E4_ver.7 | WVKQAPGQGLEWMGVINPGSGGTNYNEKFKG |
| VHhu1E4_ver.8 | WVRQAPGQGLEWMGVINPGSGGTNYNEKFKG |
| VHhu1E4_ver.9 | WVRQAPGQGLEWIGVINPGSGGTNYNEKFKG |
| | FR3 CDR3 FR4 |
| Human VH1-02 | RVTMTRDTSISTAYMELSRLRSDDTAVYYCAR----------------- |
| VHhu1E4_ver.1 | RVTMTRDTSISTAYMELSRLRSDDTAVYYCASGNYDTYWGQGTLVTVSS (1) |
| VHhu1E4_ver.2 | KVTLTADKSISTAYMELSRLRSDDTAVYYCASGNYDTYWGQGTLVTVSS (2) |
| VHhu1E4_ver.3 | KVTLTADKSISTAYMELSRLRSDDTAVYYCASGNYDTYWGQGTLVTVSS (3) |
| VHhu1E4_ver.4 | KATLTADKSISTAYMELSRLRSDDTAVYYCASGNYDTYWGQGTLVTVSS (4) |
| VHhu1E4_ver.5 | RATLTRDTSISTAYMELSRLRSDDTAVYFCASGNYDTYWGQGTLVTVSS (5) |
| VHhu1E4_ver.6 | RATLTADTSISTAYMELSRLRSDDTAVYFCASGNYDTYWGQGTLVTVSS (6) |
| VHhu1E4_ver.7 | KATLTADKSISTAYMELSRLRSDDTAVYYCASGNYDTYWGQGTLVTVSS (7) |
| VHhu1E4_ver.8 | RVTMTADKSISTAYMELSRLRSDDTAVYYCASGNYDTYWGQGTLVTVSS (8) |
| VHhu1E4_ver.9 | RATLTADKSISTAYMELSRLRSDDTAVYYCASGNYDTYWGQGTLVTVSS (9) |

In the above sequences, the human VH1-02 FR1, FR2, and FR3 sequences are designated SEQ ID NOs:61-63, respectively. The sequences for VHhu1E4_ver.1 through ver.9 (also termed hu1E4V1 through V9) are designated SEQ ID NOs:1-9, respectively, as indicated by the number in the parenthesis at the end of each sequence. The Kabat-defined HCDR1-3 sequences are designated SEQ ID NOs:33-35, respectively.

In some embodiments, the α-syn-binding proteins such as hu1E4 antibodies or antigen-binding fragments herein comprise the HCDR1-3 sequences set forth in SEQ ID NOs:33-35. In further embodiments, the humanized antibodies or antigen-binding fragments comprise the HCDR1-3 sequences set forth in SEQ ID NOs:33-35 as well as one or more (e.g., two or three) FR1-3 derived from human VH1-02. In certain embodiments, the antibodies or fragments comprise a heavy chain FR having no more than six mutations (e.g., 0, 1, 2, 3, 4, 5, or 6 mutations) relative to the corresponding FR encoded by the human germline VH1-02 gene. In certain embodiments, an antibody or antigen-binding fragment herein comprises a heavy chain FR1 having no more than two mutations, a heavy FR2 having no more than two mutations, and/or a heavy chain FR3 having no more than six mutations, relative to the corresponding FR encoded by the germline human VH1-02 gene. In some embodiments, the humanized antibody or antigen-binding fragment comprises an FR4 derived from human JH1, JH4, or JH5 gene.

In some embodiments, a humanized 1E4 antibody or antigen-binding fragment herein comprises the IMGT-defined HCDR1-3 of hu1E4V1. The IMGT-defined CDRs are italicized and underlined in the VH sequence below:

(SEQ ID NO: 1)
QVQLVQSGAEVKKPGASVKVSCKASGYTFTNYLIEWVRQAPGQGLEWMGV
INPGSGGTNYNEKFKGRVTMTRDTSISTAYMELSRLRSDDTAVYYCASGN
YDTYWGQGTLVTVSS

The IMGT-defined HCDR1-3 sequences are designated SEQ ID NOs:64-66, respectively.

In some embodiments, the α-syn-binding proteins such as humanized antibodies or antigen-binding fragments thereof comprise one or more (e.g., two or three) of the LCDRs of one of the following humanized VL sequences. Kabat-defined LCDRs are italicized; differences from LCHu1E4_VL1 are bolded and underlined.

| | |
|---|---|
| | FR1 CDR1 |
| LCHu1E4_VL1 | DIVMTQTPLSLSVTPGQPASISCRSSQSLVHSNGNTYLH |
| LChu1E4_VL2 | DVVMTQTPLSLSVTPGQPASISCRSSQSLVHSNGNTYLH |
| LChu1E4_VL3 | DVVMTQSPLSLPVTLGQPASISCRSSQSLVHSNGNTYLH |
| LChu1E4_VL4 | DVVMTQSPLSLPVTLGQPASISCRSSQSLVHSNGNTYLH |
| LChu1E4_VL5 | DVVMTQSPLSLPVTLGQPASISCRSSQSLVHSNGNTYLH |
| | FR2 CDR2 |
| LCHu1E4_VL1 | WYLQKPGQSPQLLIYKVSNRFS |
| LChu1E4_VL2 | WYLQKPGQSPQLLIYKVSNRFS |
| LChu1E4_VL3 | WFQQRPGQSPRRLIYKVSNRFS |
| LChu1E4_VL4 | WYQQRPGQSPRLLIYKVSNRFS |
| LChu1E4_VL5 | WYQQRPGQSPRLLIYKVSNRFS |
| | FR3 CDR3 FR4 |
| LCHu1E4_VL1 | GVPDRFSGSGSGTDFTLKISRVEAEDVGVYYCSQSTHVPRTFGQGTKLEIK (11) |
| LChu1E4_VL2 | GVPDRFSGSGSGTDFTLKISRVEAEDVGVYYCSQSTHVPRTFGQGTKLEIK (12) |
| LChu1E4_VL3 | GVPDRFSGSGSGTDFTLKISRVEAEDVGVYYCSQSTHVPRTFGGGTKVEIK (13) |
| LChu1E4_VL4 | GVPDRFSGSGSGTDFTLKISRVEAEDVGVYYCSQSTHVPRTFGGGTKVEIK (14) |
| LChu1E4_VL5 | GVPDRFSGSGSGTDFTLKISRVEAEDVGVYFCSQSTHVPRTFGGGTKVEIK (15) |

The sequences for LChu1E4_V1 through V5 (also termed hu1E4_VL1 through VL5) are designated SEQ ID NOs:11-15, respectively, as indicated by the number in the parenthesis at the end of each sequence. The Kabat-defined LCDR1-3 sequences are designated SEQ ID NOs:36-38, respectively.

In some embodiments, the humanized antibody or antigen-binding fragment herein comprises the IMGT-defined LCDR1-3 of hu1E4_VL1. The IMGT-defined CDRs are italicized and underlined in the VL1 sequence below:

(SEQ ID NO: 11)
DIVMTQTPLSLSVTPGQPASISCRSSQSLVHSNGNTYLHWYLQKPGQSPQ
LLIYKVSNRFSGVPDRFSGSGSGTDFTLKISRVEAEDVGVYYCSQSTHVP
RTFGQGTKLEIK

The IMGT-defined LCDR1-3 sequences are designated SEQ ID NOs:67-69. The parental mouse 1E7 CDRs (e.g., IMGT- or Kabat-defined) may be incorporated into an acceptor human kappa or lambda light chain. In some embodiments, the acceptor light chain is derived from a human kappa V2-29 (*02 or *03) gene.

In some embodiments, the α-syn-binding proteins such as humanized antibodies or antigen-binding fragments herein comprise a VH and a VL, wherein the VH comprises HCDR1-3 set forth in SEQ ID NOs:33-35 and FR1-3 derived from the human VH1-02 gene (each FR with no more than six mutations from the corresponding germline FR sequence), and the VL comprises LCDR1-3 set forth in SEQ ID NOs:36-38, respectively.

In some embodiments, the α-syn-binding proteins such as humanized antibodies or antigen-binding fragments herein comprises the HCDR1-3 and LCDR1-3 set forth in SEQ ID NOs:64-69, respectively.

In some embodiments, the α-syn-binding proteins such as humanized antibodies or antigen-binding fragments herein comprise a VH selected from SEQ ID NOs:1-9 and a VL selected from SEQ ID NOs:11-15.

In some embodiments, the humanized antibody or antigen-binding fragment herein comprises a VH and a VL set forth SEQ ID NOs:1 and 11, respectively.

In some embodiments, the humanized antibody or antigen-binding fragment herein comprises a VH and a VL set forth SEQ ID NOs:2 and 12, respectively.

In some embodiments, the humanized antibody or antigen-binding fragment herein comprises a VH and a VL set forth SEQ ID NOs:3 and 12, respectively.

In some embodiments, the humanized antibody or antigen-binding fragment herein comprises a VH and a VL set forth SEQ ID NOs:4 and 12, respectively.

In some embodiments, the humanized antibody or antigen-binding fragment herein comprises a VH and a VL set forth SEQ ID NOs:7 and 12, respectively.

In some embodiments, the humanized antibody or antigen-binding fragment herein comprises a VH and a VL set forth SEQ ID NOs:5 and 13, respectively.

In some embodiments, the humanized antibody or antigen-binding fragment herein comprises a VH and a VL set forth SEQ ID NOs:5 and 15, respectively.

In some embodiments, the humanized antibody or antigen-binding fragment herein comprises a VH and a VL set forth SEQ ID NOs:6 and 13, respectively.

In some embodiments, the humanized antibody or antigen-binding fragment herein comprises a VH and a VL set forth SEQ ID NOs:6 and 14, respectively.

In some embodiments, the humanized antibody or antigen-binding fragment herein comprises a VH and a VL set forth SEQ ID NOs:5 and 14, respectively.

In some embodiments, the humanized antibody or antigen-binding fragment herein comprises a VH and a VL set forth SEQ ID NOs:8 and 14, respectively.

In some embodiments, the humanized antibody or antigen-binding fragment herein comprises a VH and a VL set forth SEQ ID NOs:9 and 14, respectively.

In some embodiments, the humanized antibody or antigen-binding fragment herein comprise a VH and a VL, wherein the VH comprises HCDR1-3 set forth in SEQ ID NOs:33-35 and FR1-3 derived from the human VH1-02 gene (each FR with no more than six mutations from the corresponding germline FR sequence), and the VL comprises LCDR1-3 set forth in SEQ ID NOs:36-38, respectively; and wherein the VH is at least 90% (e.g., at least 91, 92, 93, 94, 95, 96, 97, 98, or 99%) homologous or identical to one of SEQ ID NOs:1-9, and/or the VL is at least 90% (e.g., at least 91, 92, 93, 94, 95, 96, 97, 98, or 99%) homologous or identical to one of SEQ ID NOs:11-15.

In some embodiments, the α-syn-binding proteins such as humanized antibodies or antigen-binding fragments herein comprises the HCDR1-3 and LCDR1-3 set forth in SEQ ID NOs:64-69, respectively; and wherein the VH is at least 90% (e.g., at least 91, 92, 93, 94, 95, 96, 97, 98, or 99%) homologous or identical to one of SEQ ID NOs:1-9, and/or the VL is at least 90% (e.g., at least 91, 92, 93, 94, 95, 96, 97, 98, or 99%) homologous or identical to one of SEQ ID NOs:11-15.

Percent (%) sequence identity or homology with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways using available computer software. Appropriate parameters for aligning sequences are able to be determined, including algorithms needed to achieve maximal alignment over the full length of the sequences being compared. In some embodiments, the query sequence has at least 70% (e.g., at least 75, 80, 85, 90, or 95%) of the length of the reference sequence. For purposes herein, sequence homology or identity may be identified by BLAST, a bioinformatics program available at the server of the United States National Center for Biotechnology Information, using default parameters.

The α-syn-binding proteins of the present disclosure, including monospecific and bispecific hu1E4 antibodies, bind specifically to disease-associated forms of α-syn, e.g., aggregated, protofibril, pre-formed fibril (PFF), or oligomeric form, with high affinity. The binding proteins may also specifically bind to disease-associated phosphorylated α-syn (e.g., α-syn phosphorylated at amino acid residue 129 (p-129 α-syn)). By binding “specifically” in the present disclosure is meant that the equilibrium dissociation constant (K_{D}) of the binding is no more than 100 nM, 50 nM, 40 nM, 30 nM, 20 nM, 10 nM, 5 nM, 2 nM, 1 nM, 0.5 nM, 0.1 nM, 0.05 nM, or 0.01 nM. K_{D} can be measured by any suitable assay. In certain embodiments, K_{D} can be measured using surface plasmon resonance (SPR) assays (e.g., using Biacore® or Octet® equipment). Specific binding of an antibody can also be demonstrated by an enzyme-linked immunosorbent assay (ELISA) using an isotype control antibody.

In some embodiments, the binding proteins such as hu1E4 antibodies or related fragments bind disease-associated forms of α-syn with a K_{D} of no more than 200 pM, 100 pM, 50 pM, 30 pM, 20 pM or 10 pM, and optionally does not bind to monomeric alpha-synuclein. In certain embodiments, the monospecific or bispecific antibodies herein bind aggregated or oligomeric α-syn with a K_{D} of 50 pM or less. In certain embodiments, the monospecific or bispecific antibodies bind aggregated or oligomeric α-syn with a K_{D} of 20 pM or less. In certain embodiments, the monospecific or bispecific antibodies bind aggregated or oligomeric α-syn with a K_{D} of 10 pM or less. In certain embodiments, the monospecific or bispecific antibodies bind aggregated or oligomeric α-syn with a K_{D} of 5 pM or less. In certain embodiments, the monospecific or bispecific antibodies bind aggregated or oligomeric α-syn with a K_{D} of 4 pM or less. In certain embodiments, the monospecific or bispecific antibodies bind aggregated or oligomeric α-syn with a K_{D} of 3 pM or less. In certain embodiments, the monospecific or bispecific antibodies bind aggregated or oligomeric α-syn with a K_{D} of 2 pM. The assay for determining the binding K_{D} can be an SPR assay performed as described in detail in Example 6 below.

Further, the binding proteins may be highly effective in promoting phagocytosis of aggregated or oligomeric α-syn by microglial cells and/or monocytes/macrophages, promoting removal of disease-associated forms (e.g., aggregated, oligomeric, and phosphorylated forms) of α-syn, and/or inhibiting propagation of diseased forms of α-syn between neurons (e.g., dopaminergic neurons).

### II. Bispecific Binding Proteins

The present disclosure also provides α-syn-binding proteins such as hu1E4 antibodies that are multispecific, e.g., bispecific. These multispecific or bispecific binding proteins are specific for both α-syn and IGF1R. The IGF1R-binding moiety allows for efficient binding of the binding protein to IGF1R and shuttling of the binding protein across the blood-brain barrier (BBB), while the α-syn-binding moiety allows for binding and clearance of aggregated or oligomeric α-syn from the brain. Thus, such multispecific, e.g., bispecific, binding proteins (e.g., antibodies) are particularly useful for the treatment of α-synucleinopathies.

In certain embodiments, the bispecific hu1E4 antibody herein crosses the BBB at a rate that is at least 2-fold, 3-fold, 4-fold, 5-fold, or 6-fold higher than a monospecific hu1E4 antibody or a chimeric 1E4 antibody, or a prior anti-α-syn antibody. In some embodiments, the bispecific hu1E4 antibody reaches the cerebral cortex, the hippocampus, and/or the substantia nigra at a rate that is at least 2-fold, 3-fold, 4-fold, 5-fold, or 6-fold higher than a monospecific hu1E4 antibody or a chimeric 1E4 antibody, or a prior anti-α-syn antibody.

The IGF1R-binding moiety may be fused to a hu1E4 antibody through a peptide linker. The IGF1R-binding moiety may, for example, fused to the N-terminus and/or C-terminus of one or both heavy chains of the hu1E4 antibody, and/or one or both light chains of the hu1E4 antibody, through a peptide linker. In some embodiments, the peptide linker may predominantly include the following amino acid residues: Gly, Ser, Ala, or Thr. The peptide linker may have a length that is adequate to link two molecules in such a way that they assume the correct conformation relative to one another so that they retain their respective desired activity. In some embodiments, the linker is 1 to 50 (e.g., 1 to 30 or 1 to 20) amino acids in length. Useful linkers include glycine-serine polymers, including for example, (GS)n, (GSGGS)n (SEQ ID NO:70), (GGGGS)n (SEQ ID NO:71), and (GGGS)n (SEQ ID NO:72), where n is an integer of at least one; glycine-alanine polymers; alanine-serine polymers; XTEN linkers; and other flexible linkers. Additional exemplary linkers for linking antibody fragments or single-chain variable fragments can include AAEPKSS (SEQ ID NO:73), AAEPKSSDKTHTCPPCP (SEQ ID NO:74), GGGG (SEQ ID NO:75), or GGGGDKTHTCPPCP (SEQ ID NO:76).

In some embodiments, the IGF1R-binding moiety comprises an antibody or an antigen-binding fragment thereof comprising HCDR1-3 set forth in SEQ ID NOs:51-53, respectively, and LCDR1-3 set forth in SEQ ID NOs:46-48, respectively.

In further embodiments, the IGF1R-binding moiety comprises an anti-IGF1R scFv comprising HCDR1-3 set forth in SEQ ID NOs:51-53, respectively, and LCDR1-3 set forth in SEQ ID NOs:46-48, respectively.

In some embodiments, the IGF1R-binding moiety comprises an antibody or an antigen-binding fragment thereof comprising a VH and a VL set forth in SEQ ID NOs:50 and 45, respectively, linked by a peptide linker, such as one as described above. In some embodiments, the peptide linker comprises SEQ ID NO:49. In certain embodiments, the moiety comprises SEQ ID NO:54. In certain embodiments, the moiety comprises a VH at least 90% homologous or identical (e.g., at least 91, 92, 93, 94, 95, 96, 97, 98, or 99%) to SEQ ID NO:50 and/or a VL at least 90% homologous or identical (e.g., at least 91, 92, 93, 94, 95, 96, 97, 98, or 99%) to SEQ ID NO:45.

In further embodiments, the IGF1R-binding moiety comprises an anti-IGF1R scFv anti-IGF1R scFv comprising a VH and a VL set forth in SEQ ID NOs:50 and 45, respectively, linked by a peptide linker, such as one as described above. In some embodiments, the peptide linker comprises SEQ ID NO:49. In certain embodiments, the scFv comprises SEQ ID NO:54. In certain embodiments, the scFv comprises a VH at least 90% homologous or identical (e.g., at least 91, 92, 93, 94, 95, 96, 97, 98, or 99%) to SEQ ID NO:50 and/or a VL at least 90% homologous or identical (e.g., at least 91, 92, 93, 94, 95, 96, 97, 98, or 99%) to SEQ ID NO:45. In certain embodiments, the scFv comprises a sequence that is at least 90% homologous or identical (e.g., at least 91, 92, 93, 94, 95, 96, 97, 98, or 99%) to SEQ ID NO:54.

In further embodiments, the multispecific binding protein is a bispecific hu1E4 antibody, with the above-described anti-IGF1R scFv fused, optionally through a peptide linker, to the C-terminus of both of its heavy chains.

In further embodiments, the multispecific binding protein is a bispecific hu1E4 antibody, with the above-described anti-IGF1R scFv fused, optionally through a peptide linker, to the C-terminus of only one of its heavy chains. In these embodiments, the bispecific antibody has two different heavy chains (one with the anti-IGF1R scFv and the other without). Thus, to promote heterodimerization of the two different heavy chains during manufacturing, mutations may be introduced to the heavy chains to physically (e.g., steric hinderance, “knobs” into “holes”) or biochemically (e.g., electrostatic interactions) deter coupling of heavy chains of the same type. For example, knobs-in-holes (KIH) mutations can be introduced to create a “knob” heavy chain” and a “hole” heavy chain that preferentially pair with each other. Exemplary KIH mutations comprise T366W in one heavy chain and T366S/L368A/Y407V in the other heavy chain (all Eu numbering). See also WO 2009/089004 and U.S. Pat. No. 8,642,745; and Brinkmann and Kontermann, MAbs. (2017) 9(2):182-212.

In some embodiments, the hu1E4 antibody is of human IgG1 isotype and contains KIH mutations, for example, having a hole heavy chain constant region comprising SEQ ID NO:55 (including the anti-IGF1R scFv sequence) and a knob heavy chain constant region comprising SEQ ID NO:39.

In some embodiments, the hu1E4 antibody is of human IgG1 isotype and contains KIH mutations and an M428L mutation, for example, having a hole heavy chain constant region comprising SEQ ID NO:56 (including the anti-IGF1R scFv sequence) and a knob heavy chain constant region comprising SEQ ID NO:41.

In particular embodiments, the hu1E4 antibody is of human IgG1 isotype and comprise a hole heavy chain comprising SEQ ID NO:57 and a knob heavy chain comprising SEQ ID NO:58, and two lights each comprising SED ID NO:59.

In particular embodiments, the hu1E4 antibody is of human IgG1 isotype and comprise a hole heavy chain comprising a sequence that is at least 90% homologous or identical (e.g., at least 91, 92, 93, 94, 95, 96, 97, 98, or 99%) to SEQ ID NO:57 and a knob heavy chain comprising a sequence that is at least 90% homologous or identical (e.g., at least 91, 92, 93, 94, 95, 96, 97, 98, or 99%) to SEQ ID NO:58, and two lights each comprising a sequence that is at least 90% homologous or identical (e.g., at least 91, 92, 93, 94, 95, 96, 97, 98, or 99%) to SED ID NO:59.

### III. Making of α-Syn-Binding Proteins

The binding proteins may be produced recombinantly using isolated nucleic acid molecules such as expression constructs encoding each chain of the proteins. Biomolecules (e.g., nucleic acid or polypeptide) molecules referred to herein as “isolated” or “purified” are those that (1) have been separated away from the biomolecules (e.g., nucleic acids of the genomic DNA or cellular RNA, or polypeptides, of their source of origin; and/or (2) do not occur in nature. The encoding sequences for each polypeptide chain may be cloned into a single vector or cloned into separate vectors.

Methods of producing proteins such as antibodies are well known. The present binding proteins such as antibodies may be produced in, e.g., mammalian host cells, using appropriate expression constructs. Mammalian cell lines available as hosts for expression include many immortalized cell lines available from the American Type Culture Collection (ATCC). These include, inter alia, Chinese hamster ovary (CHO) cells, NS0 cells, SP2 cells, HEK-293T cells, 293 Freestyle cells (Invitrogen), NIH-3T3 cells, HeLa cells, baby hamster kidney (BHK) cells, African green monkey kidney cells (COS), human hepatocellular carcinoma cells (e.g., Hep G2), A549 cells, and a number of other cell lines. Other cell lines that may be used are insect cell lines, such as Sf9 or Sf21 cells, and yeast cell lines. Cell lines may be selected based on their expression levels. The binding proteins may be isolated and purified from the host cell culture using well known methods, such as centrifugation, ultracentrifugation, protein A, protein G, protein A/G, or protein L purification, and/or ion exchange chromatography.

### IV. Pharmaceutical Compositions and Use

The present disclosure also provides pharmaceutical compositions comprising the monospecific and multispecific binding proteins herein. The pharmaceutical compositions may comprise one or more pharmaceutically acceptable excipients, carriers, or diluents. As used herein, “pharmaceutically acceptable” with reference to a carrier,” “excipient,” or “diluent” includes appropriate solvents, dispersion media, antibacterial and antifungal agents, isotonic agents, and the like. In some embodiments, the pharmaceutical composition is a sterile aqueous solution, and may comprise a buffer; a surfactant; a polyol; an antioxidant; and/or a chelating agent. In some embodiments, the pharmaceutical composition is provided in a lyophilized form and is reconstituted before administration. In certain embodiments, lyophilized antibody formulations may comprise a bulking agent.

The pharmaceutical composition may be administered to patients by parenteral administration (e.g., by injection or infusion). For example, the pharmaceutical composition may be administered by an intravenous, intracerebral, intracranial, or spinal route.

The pharmaceutical composition comprising a binding protein herein is useful in treating a human patient with, or at risk of developing, an alpha-synucleinopathy such as Parkinson's disease, dementia with Lewy bodies (DLB), multiple system atrophy (MSA), and certain forms of Alzheimer's disease (e.g., Alzheimer's disease with amygdala Lewy bodies). As used herein, the terms “treat,” “treatment,” and “treating” refers to a deliberate intervention to a physiological disease state resulting in the reduction in severity of a disease or condition; the reduction in the duration of a disease or condition; the amelioration or elimination of one or more symptoms associated with a disease or condition; or the provision of beneficial effects to a subject with a disease or condition. Treatment does not require curing the underlying disease or condition.

The pharmaceutical composition may be provided to the patient at a dosage strength and a frequency determined as appropriate by a health care provider. Therapeutically effective amounts are those sufficient to ameliorate one or more symptoms associated with the disease or affliction to be treated. A “therapeutically effective amount,” “effective dose,” “effective amount,” or “therapeutically effective dosage” of the binding protein herein protects a subject against the onset of a disease or promotes disease regression or stabilization as evidenced by a decrease in severity of disease symptoms, an increase in frequency and duration of disease symptom-free periods, or a prevention or delay of impairment or disability (e.g., cognitive ability or mobility) due to the disease affliction.

### EXAMPLES

In order that this invention may be better understood, the following examples are set forth. These examples are for purposes of illustration only and are not to be construed as limiting the scope of the invention in any manner.

### Example 1: Preparation of Chimeric Anti-α-Syn Antibody

A chimeric 1E4 (ch1E4) antibody was prepared based on the mouse anti-α-syn antibody 1E4 clone disclosed in WO 2018/128454. The amino acid sequence of the full-length human α-syn used as an antigen to obtain the 1E4 antibody is set forth in SEQ ID NO:60. The heavy chain CDR1 (HCDR1), CDR2 (HCDR2), and CDR3 (HCDR3) of mouse 1E4 are set forth in SEQ ID NOs:33 to 35 (Kabat definition), respectively, and the light chain CDR1 (LCDR1), CDR2 (LCDR2), and CDR3 (LCDR3) of mouse 1E4 are set forth in SEQ ID NOs:36 to 38, respectively.

To prepare ch1E4, the VH and VL of mouse 1E4 and human IgG1 and kappa constant regions were cloned into mammalian expression vectors (pcDNA3.4). The coding sequence for each variable region was synthesized in the form of a gBlock (M.Biotech), which was a CHO codon-optimized short nucleotide fragment. gBlocks were synthesized to overlap with the vector fragment by approximately 20 bp, and cloning was performed using the Gibson assembly method.

Next, ExpiCHO™ cells were transfected with the expression constructs using an ExpiFectamine™ CHO transfection kit (Thermo Fisher). The transfection was performed with 200 μg of DNA/200 mL ExpiCHO™ cells/1 L Erlenmeyer flask. Then, the cells were incubated for 12 days to scale up antibody production. The resulting cell culture was centrifuged and the supernatant was filtered by using a 0.2 μm pore size filter to remove suspended matter.

The filtered supernatant was purified using HiTrap™ MabSelect SuRe™ (GE Healthcare, #11-0034-94). Secondary purification was performed, when necessary, by passing the purified fraction through a HiLoad® 26/600 Superdex® 200 column (Cytiva). Mass spectroscopy was used to confirm the amino acid sequences of purified chimeric antibody.

Ch1E4 is an IgG-type monovalent antibody that includes a VH with an amino acid sequence set forth in SEQ ID NO:10 (ch1E4-VH) and a VL with an amino acid sequence set forth in SEQ ID NO:16 (ch1E4-VL).

### Example 2: Preparation of Humanized Anti-α-Syn Antibodies

Humanized versions of mouse 1E4 were also prepared. First, homology-based molecular modeling was performed based on the gene sequences of VH and VL of mouse 1E4. From this modeling, a human framework having high homology with the mouse framework was selected as an acceptor. Mouse CDRs were engrafted into the selected human frameworks to prepare a library of humanized antibodies.

To prevent loss of antigen-binding affinity during the humanization process, back mutations in the human framework may be made to introduce mouse sequences at certain positions considered to be important for antigen-binding affinity. Such back mutations may reduce the “humanness” of the humanized antibody. To evaluate the humanness of the humanized VHs and VLs generated, IMGT (%) score was calculated based on human germline sequences and a higher IMGT score means higher humanness. The results for nine versions (ver.1 through ver.9) of humanized 1E4 VH (SEQ ID NOs:1-9) and five versions (VL1 through VL5) of humanized 1E4 VL (SEQ ID NOs:11-15) are shown in Tables 1 and 2, respectively (BM: back mutation).

TABLE 1
In silico Analysis of Humanness of Humanized VHs

| Clone Name | Version name | Number of BM | IMGT Score (%) |
|---|---|---|---|
| Ch1E4 | | N/A | 70.1 |
| g0-2 | Ver. 1 | 1 | 90.7 |
| g1-2 | Ver. 2 | 5 | 85.6 |
| g2-2 | Ver. 3 | 7 | 83.5 |
| g3-2 | Ver. 7 | 7 | 83.5 |
| g4-2 | Ver. 4 | 9 | 80.4 |
| g1-g | Ver. 8 | 3 | 88.7 |
| g2-g | Ver. 9 | 6 | 85.6 |
| VH3 | Ver. 5 | 6 | 84.5 |
| VH4 | Ver. 6 | 7 | 83.5 |

TABLE 2
In silico Analysis of Humanness of Humanized VLs

| Clone Name | Version name | Number of BM | IMGT Score (%) |
|---|---|---|---|
| CP-Final-LC | | N/A | 83 |
| VL g1-1(BM2)* | VL4 | 2 | 92 |
| 2g0-2(BM0) | VL1 | 0 | 90 |
| 2g1-2(BM1) | VL2 | 1 | 89 |
| VL1(BM0) | VL3 | 0 | 94 |
| VL2(BM2)* | VL4 | 2 | 92 |
| VL4(BM3) | VL5 | 3 | 91 |

*VL g1-1 and VL2 have the same sequence.

Based on the in silico analysis, it was considered that the framework regions of Ver.1 (HC) and VL1 (LC) were the most human, due to few required back mutations and high IMGT scores.

Then, various combinations of the humanized VHs and VLs were fused to human IgG1 and kappa constant regions, respectively, producing twelve humanized 1E4 (hu1E4) antibody clones, referred to as “hu1E4(V#_VL#)” (or as “hu1E4(Ver.#_VL#)”). Table 3 lists these twelve humanized 1E4 antibodies, with the SEQ ID NO for the VH and VL shown in parentheses. The humanized antibodies were formatted as human IgG1 antibodies.

TABLE 3
Humanized 1E4 Antibodies

| Clone Name | VH (SEQ ID NO) | VL (SEQ ID NO) |
|---|---|---|
| hu1E4(V1_VL1) | Ver. 1 (1) | VL1 (11) |
| hu1E4(V2_VL2) | Ver. 2 (2) | VL2 (12) |
| hu1E4(V3_VL2) | Ver. 3 (3) | VL2 (12) |
| hu1E4(V4_VL2) | Ver. 4 (4) | VL2 (12) |
| hu1E4(V7_VL2) | Ver. 7 (7) | VL2 (12) |
| hu1E4(V5_VL3) | Ver. 5 (5) | VL3 (13) |
| hu1E4(V5_VL5) | Ver. 5 (5) | VL5 (15) |
| hu1E4(V6_VL3) | Ver. 6 (6) | VL3 (13) |
| hu1E4(V6_VL4) | Ver. 6 (6) | VL4 (14) |
| hu1E4(V5_VL4) | Ver. 5 (5) | VL4 (14) |
| hu1E4(V8_VL4) | Ver. 8 (8) | VL4 (14) |
| hu1E4(V9_VL4) | Ver. 9 (9) | VL4 (14) |

### Example 3: Preparation of Anti-α-Syn/IGF1R Bispecific Antibodies

Next, bispecific antibodies against α-syn and IGF1R were generated. The IGF1R-binding portion promotes receptor-mediated transcytosis through the blood-brain barrier and can be fused to the C-terminus of one or both heavy chains of the anti-α-syn antibody. In this Example, each bispecific antibody had a structure in which the N-terminal VL of an IGF1R-binding scFv was fused to an anti-α-syn antibody with knob-in-hole mutations in the Fc domain. The anti-IGF1R scFv was fused only to the heavy chain containing the “hole” mutations (“hole heavy chain”), via a peptide linker.

The anti-IGF1R scFv was based on an F06(de2)(StoP) clone described in WO 2020/251316. The monovalent, conjugated F06(de2)(StoP) in scFv form is referred to as “Grabody B” herein (SEQ ID NO:54). Grabody B is comprised of, from N-terminus to C-terminus, a VL (SEQ ID NO:45), a (G₄S)₄ linker (SEQ ID NO:49), and a VH (SEQ ID NO:50). The LCDR1-3 of Grabody B are set forth in SEQ ID NOs:46-48, respectively, and its HCDR1-3 are set forth in SEQ ID NOs:51-53, respectively.

The anti-α-syn/IGF1R bispecific antibodies were asymmetric structurally. They each included one anti-α-syn IgG1 heavy chain linked at its C-terminus to the N-terminus of Grabody B through a peptide linker (G₄S)₃ (SEQ ID NO:44), and one anti-α-syn IgG1 heavy chain not linked to Grabody B. To promote this heterodimeric heavy chain combination during antibody production, knob-in-hole (KIH) mutations were introduced into the IgG1 heavy chain Fc domain. The “hole” mutations were T366S, L368A, and Y406V in the CH3 domain, and the “knob” mutation was replaced with T366W in the CH3 domain (all per Eu numbering). The sequence of the IgG1 heavy chain constant region into which the knob mutations have been introduced is set forth in SEQ ID NO:39; the sequence of the IgG1 heavy chain constant region into which the hole mutations have been introduced is set forth in SEQ ID NO:40. The sequence of the “knob” IgG1 heavy chain constant region into which an M428L mutation has been further introduced is set forth in SEQ ID NO:41; and the sequence of the “hole” IgG1 heavy chain constant region into which an M428L mutation has been further introduced is set forth in SEQ ID NO:42. The M428L mutation was introduced to increase serum half-life of the antibody via increasing its affinity for FcRn and therefore recycling in vivo.

Unless otherwise indicated, the hIgG1 heavy chain constant region (knob) (M428L) (SEQ ID NO:41) and the hIgG1 heavy chain constant region (hole) (M428L) (SEQ ID NO:42) were used to construct the bispecific antibodies. A human kappa light chain (SEQ ID NO:43) was used as the source for the light chain.

The hIgG1 heavy chain constant region linked to the anti-IGF1R scFv (hIgG1 (constant)-Grabody B (hole)) has a sequence set forth in SEQ ID NO:55. The hIgG1 heavy chain constant region not linked to the anti-IGF1R scFv (hIgG1 (constant) (knob)) has a sequence set forth in SEQ ID NO:39. The hIgG1 heavy chain constant regions—hIgG1 (constant, M428L)-Grabody B (hole) and hIgG1 (constant, M428L) (knob) have sequences set forth in SEQ ID NOs:56 and 41, respectively, where both heavy chains contain the M428L mutation.

The bispecific antibodies were produced by transfecting ExpiCHO™ cells (Gibco) with expression vectors encoding the hole heavy chain, the knob heavy chain, and the light chain at a ratio of 0.5:0.5:1. One day before transfection, the CHO cells were seeded in an ExpiCHO™ expression medium (Gibco) at a concentration of 3×10⁶ to 4×10⁶ viable cells/mL, and then cultured at 8% CO₂, 37° C., and 120 rpm for one day.

On the day of transfection, the cells were diluted with fresh medium from a concentration of 7×10⁶ to 10×10⁶ viable cells/mL with a 95% or higher viability, to a concentration of 6×10⁶ viable cells/mL.

For transfection of the parental cells, an ExpiFectamine™ CHO Transfection Kit (Gibco) was used. Plasmid DNA and ExpiFectamine™ CHO Reagent were mixed to create an ExpiFectamine™ CHO/plasmid DNA complex. The complex was seeded in cold OptiPRO™ SFM medium (Gibco). The resulting mixture was kept at room temperature for 5 minutes and added to the parental cells. One day after transfection, ExpiFectamine™ CHO Enhancer and ExpiCHO™ Feed were added to the transfected cells. Five days after transfection, a second volume of the Feed was added to the cell culture. After culturing for ten days under conditions of 8% CO₂, 37° C., and 120 rpm, the cell culture was transferred to a centrifuge bottle, centrifuged at 4° C. and 6,500 rpm for 30 minutes. The supernatant was filtered through a 0.2 μm filter to remove suspended matter and then further purified to obtain the bispecific antibody.

### Example 4: Sandwich ELISA Test for Evaluation of Antibody Affinity for α-Syn Pre-Formed Fibrils

Sandwich ELISA was to analyze the binding affinities of 1E4 chimeric and humanized antibodies to α-syn pre-formed fibrils (PFF), using human α-syn PFF from StressMarq (Human Alpha Synuclein Pre-formed Fibrils Type II, SPR-317). Specifically, the antibodies were diluted with PBS by six-fold serial dilution from 400 nM to 0.009 nM. The diluted antibodies were added to a 96-well plate at 100 μL/well for coating. The plate was sealed and incubated at 4° C. for 16 hours. After washing with phosphate-buffered saline with Tween® (PBS-T; 0.05% Tween® 20) five times, the plate was blocked with 5% BSA in PBS 200 μL/well at 37° C. for 2 hours. After washing with PBS-T five times, α-syn PFF (2 μg/mL, 100 μL/well in 2% BSA in PBS) was added to the plate and incubated at 37° C. for 2 hours. After washing with PBS-T five times, 7B7-biotin antibody (anti-α-syn antibody; 1 μg/mL/100 μL/well, in 2% BSA in PBS) was added and incubated at 37° C. for 2 hours. After washing with PBS-T five times, the plate was incubated with horseradish peroxidase (HRP)-bound streptavidin in PBS with 2% BSA (1:5000, 1 mg/mL stock, 20 ng/well, 100 μL/well) at 37° C. for 1 hour. After washing with PBS-T five times, 3,3′,5,5′-tetramethylbenzidine (TMB) (100 μL/well) was added and incubated at room temperature for 5 minutes. The reaction was stopped by the addition of 0.5 N H₂SO₄ (50 μL/well). Optical absorbance at 450 nm was measured by using a plate reader, with absorbance at 650 nm subtracted from the reading.

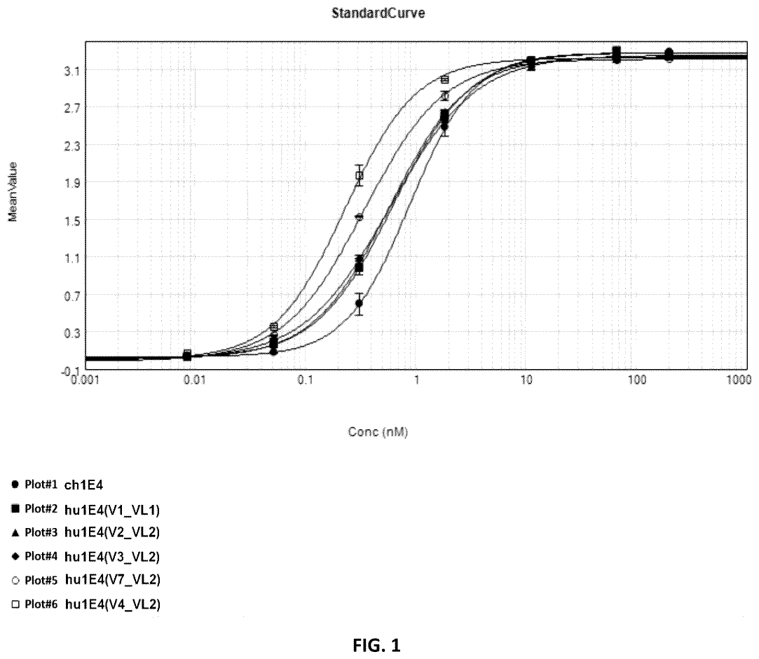

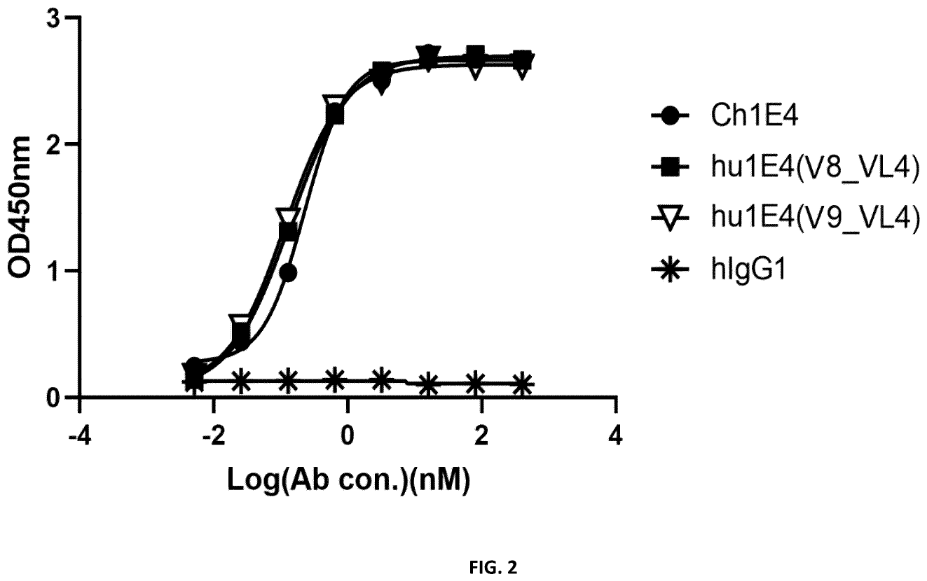

FIG. 1 and FIG. 2 show the results of binding of chimeric and monospecific humanized 1E4 antibodies to the PFF. Table 4 and Table 5 below show the EC₅₀ values of the chimeric 1E4 antibody and select monospecific humanized 1E4 (hu1E4) antibodies from the ELISA.

TABLE 4
EC₅₀ of Binding of Chimeric and Humanized
1E4 Antibodies to α-syn PFF

| Clone Name | EC₅₀ (nM) |
|---|---|
| ch1E4 | 0.876 |
| hu1E4(V1 VL1) | 0.619 |
| hu1E4(V2 VL2) | 0.570 |
| hu1E4(V3 VL2) | 0.577 |
| hu1E4(V7 VL2) | 0.339 |
| hu1E4(V4 VL2) | 0.228 |

TABLE 5
EC₅₀ of Binding of Additional Humanized
1E4 Antibodies to α-syn PFF

| Clone Name | EC₅₀ (nM) |
|---|---|
| ch1E4 | 0.209 |
| hu1E4(V8 VL4) | 0.139 |
| hu1E4(V9 VL4) | 0.114 |
| hIgG1 | Not measurable |

These data show that all of the tested chimeric and humanized 1E4 antibodies exhibited high binding affinity for α-syn PFF, and the humanized antibodies exhibited higher binding affinity than the chimeric antibody. Since the chimeric antibody contains the VH and VL of the parental mouse 1E4 antibody, these results indicate that the humanization process improved the antigen-binding affinity of the 1E4 antibody.

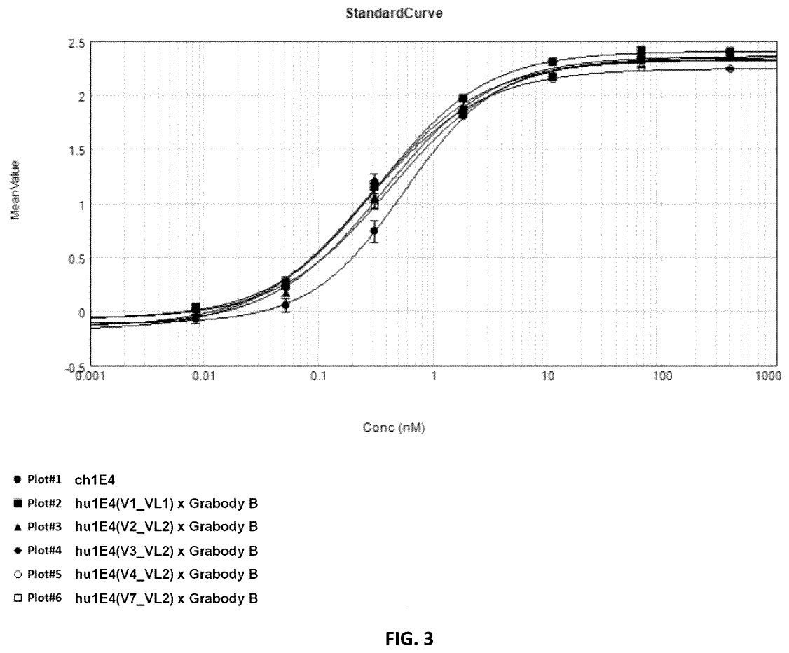

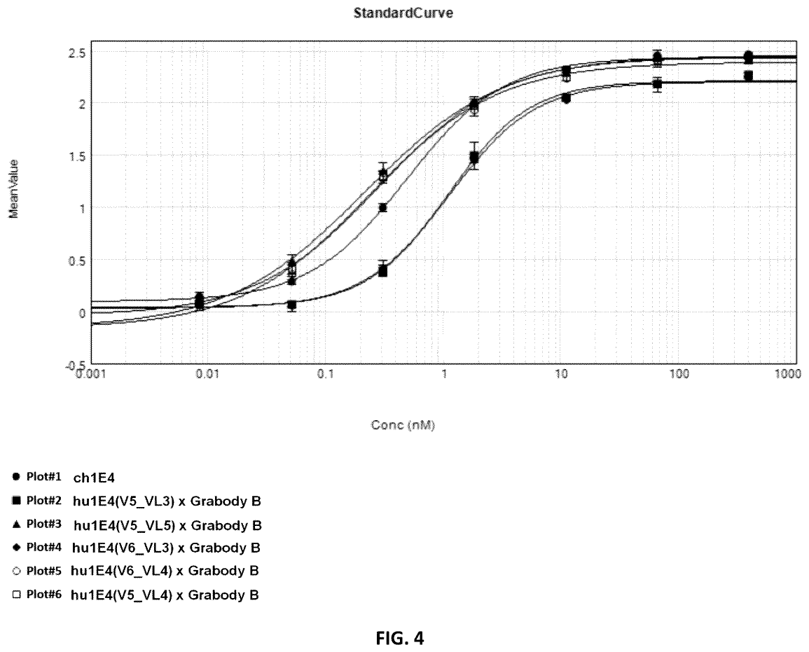

FIG. 3 and FIG. 4 show the ELISA results of chimeric and bispecific 1E4 antibodies. Table 6 and Table 7 below show the EC₅₀ values of these antibodies from the sandwich ELISA.

TABLE 6
EC₅₀ of Binding of Bispecific Anti-α-syn/IGF1R
Antibodies to α-Syn PFF

| Antibody Clone Name | EC₅₀ (nM) |
|---|---|
| ch1E4 | 0.554 |
| hu1E4(V1 VL1) × Grabody B | 0.334 |
| hu1E4(V2 VL2) × Grabody B | 0.364 |
| hu1E4(V3 VL2) × Grabody B | 0.285 |
| hu1E4(V4 VL2) × Grabody B | 0.264 |
| hu1E4(V7 VL2) × Grabody B | 0.418 |

TABLE 7
EC₅₀ of Binding of Additional Bispecific Antibodies
to α-Syn PFF

| Antibody Clone Name | EC₅₀ (nM) |
|---|---|
| ch1E4 | 0.484 |
| hu1E4(V5 VL3) × Grabody B | 1.098 |
| hu1E4(V5 VL5) × Grabody B | 0.215 |
| hu1E4(V6 VL3) × Grabody B | 1.118 |
| hu1E4(V6 VL4) × Grabody B | 0.233 |
| hu1E4(V5 VL4) × Grabody B | 0.284 |

The above data show that all of the tested bispecific antibodies exhibited high binding affinity for α-syn PFF, with some exhibiting higher binding affinity than the chimeric antibody.

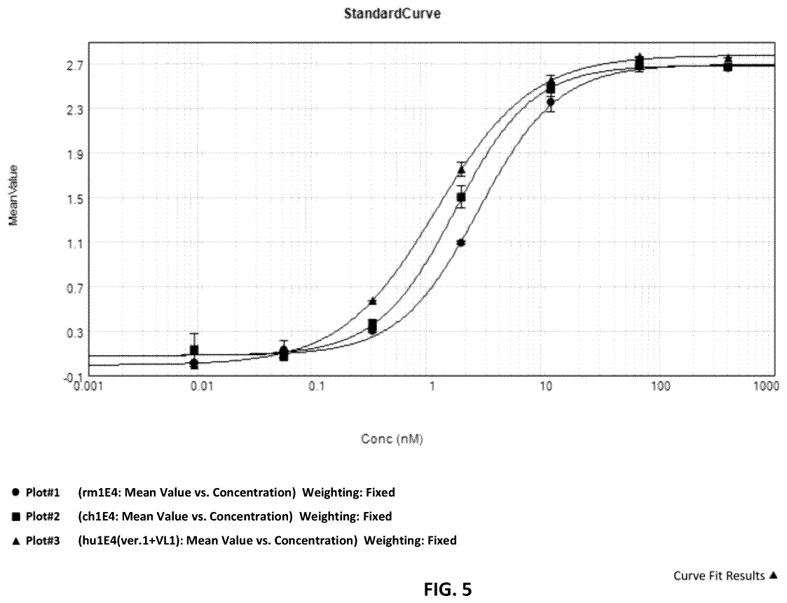

Next, the binding affinity for α-syn PFF was evaluated using sandwich ELISA and directly compared among mouse 1E4 (rm1E4), chimeric 1E4 (ch1E4), and monospecific humanized 1E4(V1_VL1), as described above. As shown in FIG. 5 and Table 8 below, the hu1E4 antibody exhibited higher affinity for α-syn PFF compared to mouse or chimeric 1E4.

TABLE 8
Comparison of Mouse, Chimeric, and
Humanized 1E4 Antibodies

| Antibody Clone | EC₅₀ (nM) |
|---|---|
| rm1E4 | 2.588 |
| ch1E4 | 1.609 |
| hu1E4(V1_VL1) | 1.098 |

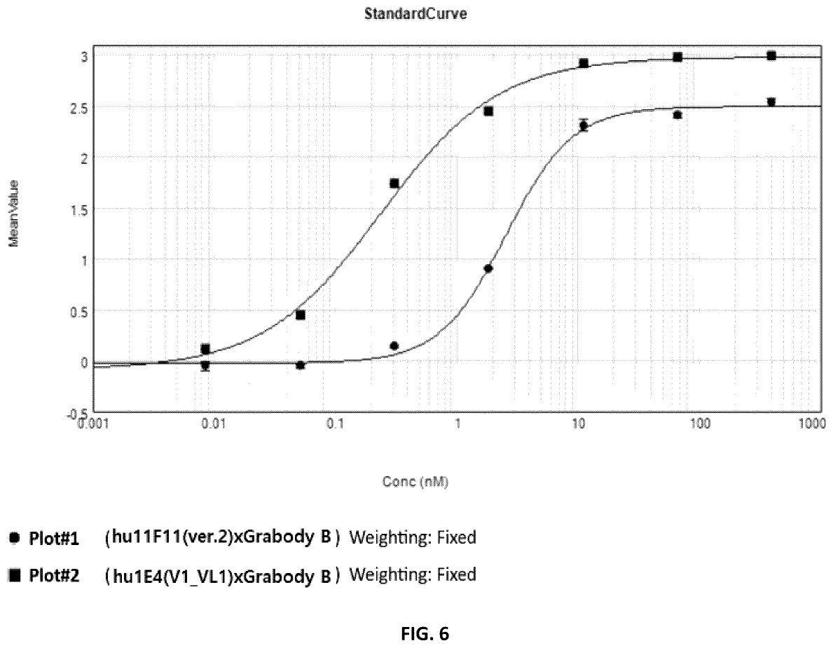

The binding affinity for α-syn PFF was also evaluated by sandwich ELISA and compared between hu1E4(V1_VL1) and a humanized version of another mouse anti-α-syn antibody, hu11F11(ver.2) (see WO 2019/098763). Both antibodies were configured to include a fused Grabody B as described in Example 3. As shown in FIG. 6 and Table 9, the bispecific “hu1E4(V1_VL1)×Grabody B” antibody exhibited much higher affinity for α-syn PFF than the bispecific “hu11F11(ver.2)×Grabody B” antibody.

TABLE 9
Comparison of Bispecific Hu1E4 and Hu11F11 Antibodies

| Antibody Clone Name | EC₅₀ (nM) |
|---|---|
| hu11F11(ver. 2) × Grabody B | 2.537 |
| hu1E4(V1_VL1) × Grabody B | 0.236 |

### Example 5: Sandwich ELISA for Evaluation of Antibody Affinity for Oligomeric α-Syn

Unlike α-syn PFF, dopamine HCl-stabilized α-syn oligomers have few β-sheets and are considered to be a different type of aggregate than PFF because the oligomers have a small and globular shape. Antibodies that bind α-syn PFF and dopamine HCl-stabilized α-syn oligomers may act widely on various types of aggregates present in the brains of patients at various stages of synucleinopathies such as Parkinson's disease.

To evaluate the binding affinity of hu1E4 antibodies for oligomeric α-syn and compare them to existing anti-α-syn antibodies, a sandwich ELISA was performed as described above, using monospecific hu1E4(V1_VL1), bispecific hu1E4 (ver.1_VL1)×Grabody B, monospecific hu11F11(ver.2), the chimeric 9E4 antibody (Roche), the NI-202 antibody (Biogen), and hy1E4, which is a mouse 1E4 antibody. Human α-syn oligomers from StressMarq (Dopamine HCL Stabilized Human Recombinant Alpha Synuclein Oligomers, SPR-466) was used as the oligomeric α-syn in the assay.

In this sandwich ELISA, test antibodies were diluted with PBS by five-fold serial dilution from 80 nM to 0.005 nM. The serially diluted antibodies were added to a 96-well plate at 100 μL/well for coating. The plate was sealed and incubated at 4° C. for 16 hours. After washing with PBS-T five times, the plate was blocked with 5% BSA in PBS 200 μL/well at 37° C. for 2 hours. After washing with PBS-T five times, oligomeric α-syn (2 μg/mL, 100 μL/well in 2% BSA in PBS) was added and incubated at 37° C. for 2 hours. After washing with PBS-T five times, 7B7-biotin antibody (1 μg/mL, 100 μL/well, in 2% BSA in PBS) were added and incubated at 37° C. for 2 hours. After washing with PBS-T five times, the plate was incubated with Streptavidin-HRP in 2% BSA in PBS (1:5000, 1 mg/mL stock, 20 ng/well, 100 μL/well) at 37° C. for 1 hour. After washing with PBS-T five times, TMB (100 μL/well) was added and incubated at RT for 5 minutes. The reaction was stopped by the addition of 0.5 N H₂SO₄ (50 μL/well). Optical absorbance at 450 nM was measured by using a plate reader, with absorbance at 650 nm subtracted from the reading.

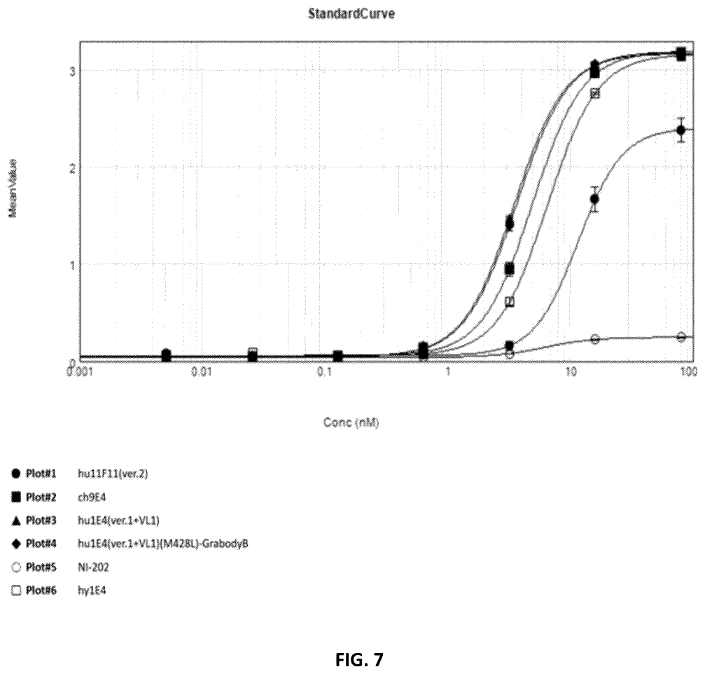

As shown in FIG. 7 and Table 10 below, hu1E4(V1_VL1) and its bispecific version bound α-syn oligomers better than all other comparators tested.

TABLE 10
Comparison of Binding Affinity for Oligomeric α-Syn

| Antibody Clone Name | Source | EC₅₀ (nM) | Saturation point |
|---|---|---|---|
| hu11F11 | Applicant | 14.11 | 2.821 |
| ch9E4 | Roche | 4.925 | 3.196 |
| hu1E4(V1_VL1) | Applicant | 3.455 | 3.159 |
| hu1E4(V1_VL1) × | | 3.624 | 3.181 |
| Grabody B | | | |
| NI-202 | Biogen | n/m* | n/m |
| hy1E4 | Applicant | 6.559 | 3.175 |

*n/m: not measurable.

These results suggest that both monospecific and bispecific forms of a humanized 1E4 antibody of the present invention exhibited superior binding affinity as compared to existing commercialized anti-α-syn antibodies.

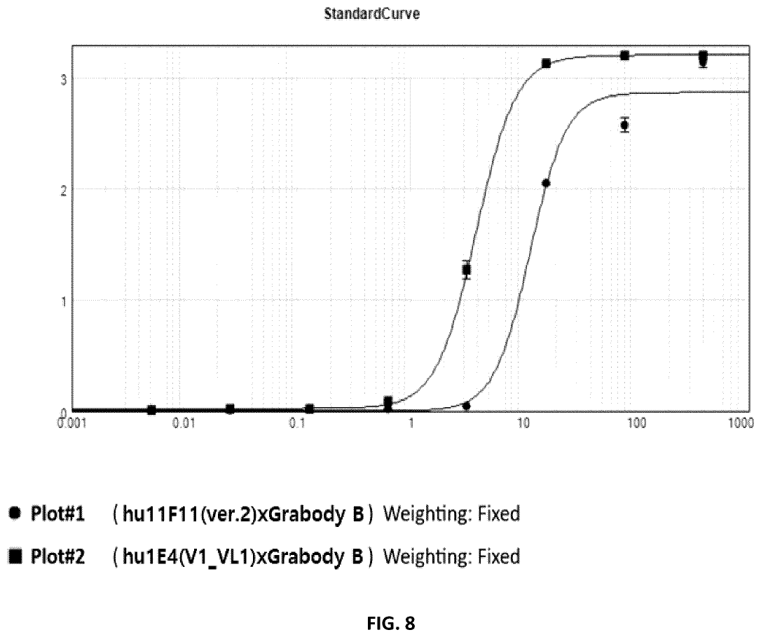

Next, the binding affinity for oligomeric α-syn was evaluated and compared between hu1E4(V1_VL1) and hu11F11(ver.2) in a bispecific antibody format with Grabody B. As shown in FIG. 8 and Table 11, the “hu1E4(V1_VL1)×Grabody B” bispecific antibody exhibited higher binding for oligomeric α-syn than the “hu11F11(ver.2)×Grabody B” bispecific antibody.

TABLE 11
Comparison of Bispecific Antibodies for Binding to Oligomeric α-Syn

| Antibody | EC₅₀ (nM) |
|---|---|
| hu11F11(ver. 2) × Grabody B | 11.51 |
| hu1E4(V1_VL1) × Grabody B | 3.82 |

### Example 6: Biacore® Evaluation of Antibody Binding Affinity for α-Syn PFF

Surface plasmon resonance (SPR) analysis was performed to quantitatively analyze the binding affinities of the chimeric and humanized anti-α-syn 1E4 antibodies. The equipment used was Biacore® (GE Healthcare, Model T200). Protein A (GE Healthcare, Cat: 29-1275-56) was used as a chip, 10 mM glycine-HCl pH 1.5 (GE Healthcare, Cat: BR-1003-54) was used as a regeneration buffer, and HBS-EP was used as a running buffer and an analyte dilution and sample dilution buffer. The antibody was diluted with 1X HBS-EP, and α-syn fibril protein solutions (PFF, 2.6 mg/mL) (analyte) were serially diluted two-fold to analyze six samples at concentrations of 0, 0.15625, 0.3125, 0.625, 1.25, and 2.5 nM. A capture phase was carried out at a contact time of 60 seconds and a flow rate of 30 μL/min was used for a stabilization period of 60 seconds, with the target resonance units (RU) of fibril set to 10 (theoretical). In an association phase, the association time was set to 60 seconds and a flow rate was set to 60 μL/min. In a dissociation phase, the dissociation time was set to 180 seconds, and a flow rate was set to 60 μL/min. In a regeneration phase, the flow rate was set to 30 μL/min, and the regeneration time was set to 60 seconds. A 1:1 binding model was used for fitting, and the Biacore® T200 Evaluation software (GE Healthcare) was used for the evaluation.

As shown in Table 12, some humanized 1E4 clones exhibited a level of binding affinity comparable to the chimeric 1E4, and some clones such as hu1E4(V1_VL1) even exhibited superior binding affinity compared to the chimeric 1E4. Hu1E4(V1_VL1) has a better k_{d} value than ch1E4, which means that once hu1E4(V1_VL1) binds to α-syn, it maintains the binding and does not dissociate easily. This can be also evidenced by high affinity (low K_{D} value). Because it is usually difficult to maintain the initial binding affinities of the mouse and chimeric antibodies during humanization, these results are surprising.

TABLE 12
Binding Affinities and Kinetics of Chimeric and Humanized 1E4 Antibodies

| Clone | Run | kₐ | k_{d} | K_{D} | Rₘₐₓ | Chi² |
|---|---|---|---|---|---|---|
| Name | (N) | (1/Ms, ×10⁷) | (1/s, ×10⁻⁴) | (M, ×10⁻¹¹) | (RU) | (Min~Max) |
| ch1E4 | 1 | 1.042 | 5.403 | 5.188 | 11.39 | 0.026 |
| hu1E4 | 2 | 1.591 ± 0.037 | 4.391 ± 0.426 | 2.763 ± 0.332 | 25.20 ± 1.59 | 0.367~0.466 |
| (V1_VL1) | | | | | | |
| hu1E4 | 2 | 1.677 ± 0.001 | 8.543 ± 0.259 | 5.095 ± 0.149 | 23.36 ± 0.63 | 0.351~0.391 |
| (V8_VL4) | | | | | | |
| hu1E4 | 2 | 1.424 ± 0.011 | 13.665 ± 0.389 | 9.603 ± 0.347 | 24.91 ± 0.52 | 0.266~0.285 |
| (V9_VL4) | | | | | | |

The hu1E4(V1_VL1) clone was formatted into an anti-α-syn/IGF1R bispecific antibody with Grabody B. Then, the binding affinities of the antibodies for α-syn PFF were tested as described above. As shown in Table 13, it was confirmed that the hu1E4(V1_VL1) monospecific antibody and bispecific antibody (BsAb) had similar K_{D} values in a 1:1 binding model. These data suggest that the Grabody B moiety did not have a negative effect on the binding affinity of hu1E4 for α-syn PFF when it was used as a bispecific antibody partner.

TABLE 13
Comparison of Monospecific and Bispecific hu1E4 Antibodies

| Clone | | Run | kₐ | k_{d} | K_{D} | Rₘₐₓ | Chi² |
|---|---|---|---|---|---|---|---|
| Name | Format | (N) | (1/Ms, ×10⁷) | (1/s, ×10⁻⁴) | (M, ×10⁻¹¹) | (RU) | (Min~Max) |
| hu1E4 | mAb | 2 | 1.376 ± | 4.151 ± | 3.021 ± | 20.85 ± 0.75 | 0.165~0.187 |
| (V1_VL1) | | | 0.066 | 0.083 | 0.206 | | |
| hu1E4 | BsAb | 2 | 1.448 ± | 4.231 ± | 2.927 ± | 19.72 ± 0.60 | 0.143~0.185 |
| (V1_VL1) × | | | 0.073 | 0.064 | 0.191 | | |
| Grabody | | | | | | | |

Various humanized 1E4 clones were prepared as anti-α-syn/IGF1R BsAbs with Grabody B, and their affinity for PFF was tested as described above and compared with the SPR results of ch1E4. As shown in Table 14, the bispecific humanized 1E4 antibodies also exhibited binding affinities comparable to the chimeric 1E4 in a 1:1 binding model. Some clones, such as hu1E4(V1_VL1), exhibited superior binding affinity in the BsAb format, compared to the monospecific chimeric 1E4 antibody.

TABLE 14
Binding Affinities and Kinetics of Chimeric 1E4 and Hu1E4 BsAbs

| | Run | kₐ | k_{d} | K_{D} | Rₘₐₓ | |
|---|---|---|---|---|---|---|
| Antibody Name | (N) | (1/Ms) | (1/s) | (M) | (RU) | Chi² |
| ch1E4 | 1 | 1.042 × 10⁷ | 5.403 × 10⁻⁴ | 5.188 × 10⁻¹¹ | 11.39 | 0.026 |
| hu1E4(V1_VL1) × | 1 | 1.143 × 10⁷ | 3.218 × 10⁻⁴ | 2.814 × 10⁻¹¹ | 15.58 | 0.065 |
| Grabody B | | | | | | |
| hu1E4(V3_VL2) × | 1 | 1.010 × 10⁷ | 9.918 × 10⁻⁴ | 9.815 × 10⁻¹¹ | 15.79 | 0.066 |
| Grabody B | | | | | | |
| hu1E4(V7_VL2) × | 1 | 1.049 × 10⁷ | 1.498 × 10⁻³ | 1.427 × 10⁻¹⁰ | 12.50 | 0.069 |
| Grabody B | | | | | | |
| hu1E4(V5_VL4) × | 1 | 9.959 × 10⁶ | 1.599 × 10⁻³ | 1.666 × 10⁻¹⁰ | 11.26 | 0.150 |
| Grabody B | | | | | | |
| hu1E4(V5_VL5) × | 1 | 1.135 × 10⁷ | 6.700 × 10⁻⁴ | 5.904 × 10⁻¹¹ | 13.59 | 0.077 |
| Grabody B | | | | | | |
| hu1E4(V6_VL4) × | 1 | 4.987 × 10⁶ | 9.324 × 10⁻⁴ | 1.870 × 10⁻¹⁰ | 15.85 | 0.139 |
| Grabody B | | | | | | |

To confirm the superiority of hu1E4(V1_VL1) over the previous anti-α-syn humanized antibody hu11F11, SPR analysis was used to quantitatively analyze the binding affinities of hu1E4(V1_VL1)×Grabody B and hu11F11(ver.2)×Grabody B for α-syn PFF. The equipment used was Biacore®. Anti-hFab (Sigma, 15260-1ML) diluted in acetate buffer (pH 5.0) was immobilized on the surface of an CM3 chip (GE Healthcare) by amine coupling. Then, test articles were diluted with HBS-EP buffer and the diluted samples were captured at about 10 RU (for hu1E4(V1_VL1)×Grabody B) or 50 RU (for hu11F11(ver.2)×Grabody B) under 30 μL/min on the anti-hFab chip surface. α-syn PFF was diluted with HBS-EP buffer by 3-fold serial dilution at concentrations of 0, 0.012, 0.037, 0.111, 0.333, and 1 nM for hu1E4(V1_VL1)×Grabody B, or 0, 0.111, 0.333, 1, 3, and 9 nM for hu11F11(ver.2)×Grabody B. Each diluted α-syn PFF solution was injected at 60 μL/min onto the antibody-captured chip surface for 60 sec, followed by washing with HBS-EP (1X) buffer on the surface for 180 sec. All procedures in association and dissociation phases were done in single-cycle kinetics. A 1:1 binding model was used for fitting, and the Biacore® T200 Evaluation software was used for the evaluation.

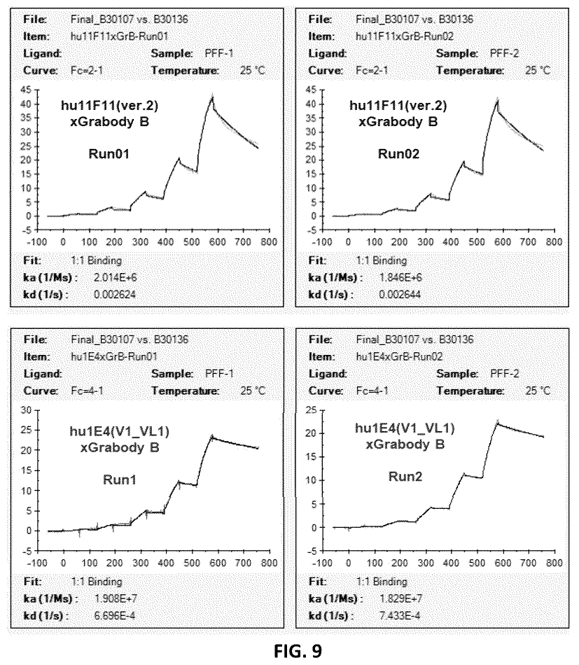

The results shown in Table 15 and FIG. 9 indicate that hu1E4(V1_VL1) BsAb had much better α-syn PFF binding affinity than hu11F11(ver.2) bispecific antibody.

TABLE 15
Comparison of Hu1E4 and Hu11F11 BsAbs

| | | | | | Rₘₐₓ | Chi² |
|---|---|---|---|---|---|---|
| BsAb Name | N | kₐ (1/Ms) | k_{d} (1/s) | K_{D} (M) | (RU) | (Min~Max) |
| hu11F11(ver.2) × | 2 | (1.930 ± 0.119) × | (2.634 ± 0.014) × | (1.368 ± 0.092) × | 54.62 ± 0.42 | 0.499~0.570 |
| Grabody B) | | 10⁶ | 10⁻³ | 10⁻⁹ | | |
| hu1E4(V1_VL1) × | 2 | (1.869 ± 0.056) × | (7.065 ± 0.521) × | (3.786 ± 0.392) × | 28.73 ± 0.38 | 0.027~0.096 |
| Grabody B | | 10⁷ | 10⁻⁴ | 10⁻¹¹ | | |

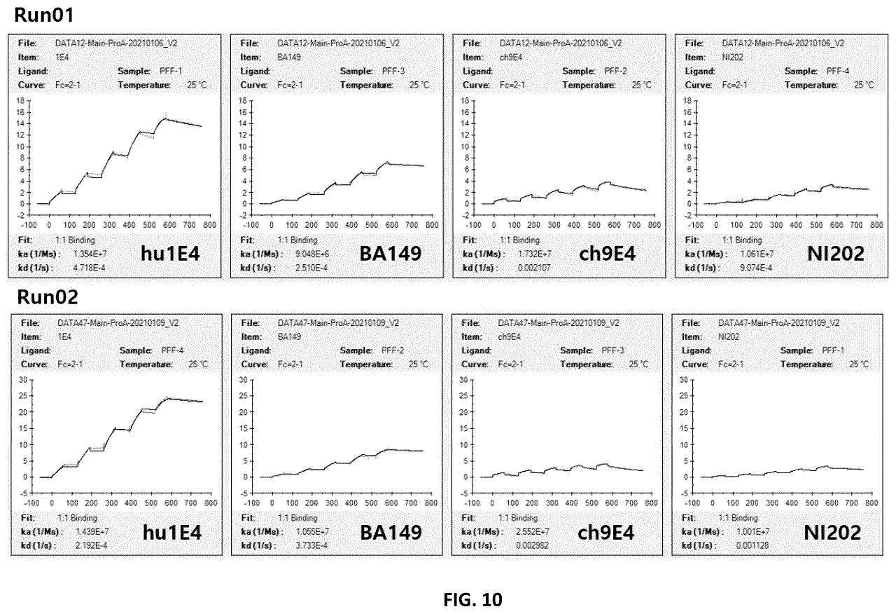

The binding affinity for α-syn PFF was evaluated and compared between hu1E4(V1_VL1)×Grabody B and other commercially available anti-α-syn antibodies through SPR analysis as described above. The data in Table 16 and FIG. 10 show that hu1E4(V1_VL1)×Grabody B had an equilibrium dissociation constant (K_{D}) of 2.5×10⁻¹¹ M for α-syn PFF, thus exhibiting superior α-syn PFF binding affinity as compared to existing anti-α-syn antibodies.

TABLE 16
Comparison of Hu1E4 BsAb and Commercially Available Antibodies

| | | kₐ | k_{d} | K_{D} | Rₘₐₓ | Chi² |
|---|---|---|---|---|---|---|
| Antibody | N | (1/Ms, ×10⁷) | (1/s, ×10⁻⁴) | (M, ×10⁻¹¹) | (RU) | (Min~Max) |
| Hu1E4(V1_VL1) × | 2 | 1.397 ± | 3.455 ± 1.786 | 2.505 ± 1.387 | 19.990 ± 6.661 | 0.098~0.288 |
| Grabody B | | 0.060 | | | | |
| BA149 | 2 | 0.980 ± | 3.122 ± 0.865 | 3.156 ± 0.540 | 8.375 ± 1.098 | 0.022~0.096 |
| (BioArctic) | | 0.106 | | | | |
| ch9E4 | 2 | 2.142 ± | 25.445 ± 6.187 | 11.920 ± 0.339 | 3.559 ± 0.033 | 0.025~0.032 |
| (Roche/Prothena) | | 0.580 | | | | |
| NI202 (Biogen) | 2 | 1.031 ± | 10.177 ± 1.560 | 9.913 ± 1.919 | 3.244 ± 0.006 | 0.010~0.011 |
| | | 0.042 | | | | |

These data demonstrate that even in a BsAb form, the humanized antibodies of the present disclosure have superior α-syn PFF binding affinity as compared to existing anti-α-syn monospecific antibodies.

### Example 7: Biacore® Evaluation of Antibody Binding Affinity for α-Syn Monomers

The binding affinities of hu1E4(V1_VL1) and hu1E4(V8_VL4) for monomeric α-syn were evaluated and compared with the existing anti-α-syn antibody ch9E4 (Roche/Prothena; prasinezumab). An unrelated human IgG1 (hIgG1) was used as a negative control. SPR analysis was used to quantitatively analyze the binding affinities to monomeric α-syn (Active Human Recombinant Alpha Synuclein Protein Monomer type II, StressMarq, SPR-316). The equipment used was Biacore® T200. The anti-α-syn monospecific antibody or the anti-α-syn/IGF1R bispecific antibody was diluted with 1X HBS-EPT. The diluted antibody samples were captured at about 800 RU under 30 μL/min on the surface of a Protein A chip (GE Healthcare, Cat: 29-1275-56). α-synuclein monomer was diluted with 1X HBS-EP buffer from 50 nM to 3.125 nM by two-fold serial-dilution. Each diluted monomer sample was injected at 60 μL/min onto the Protein A chip surface for 60 seconds, followed by washing with 1X HBS-EP buffer. The dissociation rate was evaluated for 180 seconds. All procedures in association/dissociation phases were done in single cycle kinetics. The chip surface was regenerated by injecting 10 mM glycine-HCl pH 1.5 (GE Healthcare) onto the chip surface for 60 seconds at 30 μL/min, removing residual α-syn monomer/antibody complex. A 1:1 binding model was used for fitting, and the Biacore® T200 Evaluation software was used for evaluation.

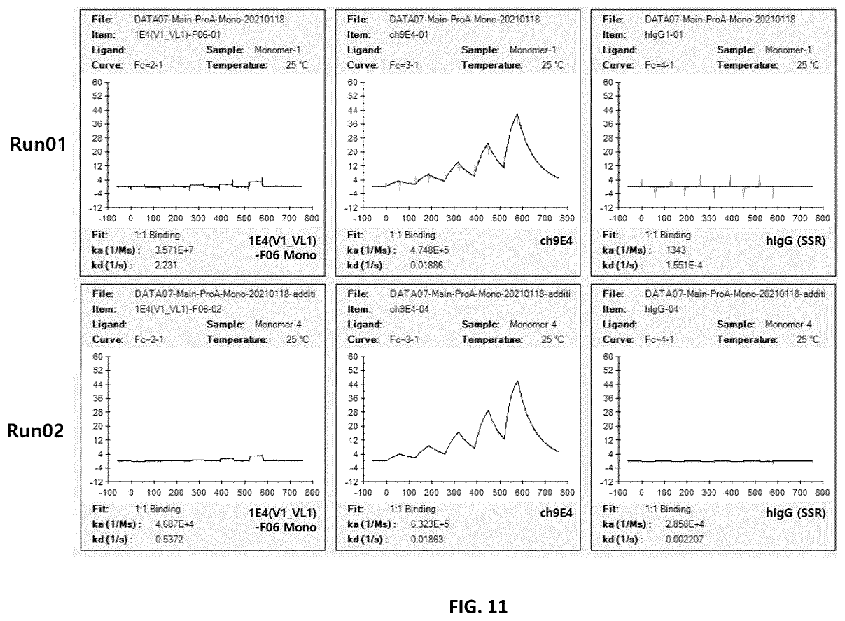

The data in Tables 17-19 and FIG. 11 show that the monospecific and bispecific hu1E4 antibodies of the present disclosure exhibited very low binding affinity for monomeric α-syn. By contrast, the ch9E4 antibody bound with strong affinity for the monomer.

TABLE 17
Comparison of Ch9E4 and Monospecific Hu1E4(V1_VL1) Antibodies

| | Run | kₐ | k_{d} | K_{D} | Rₘₐₓ | Chi² |
|---|---|---|---|---|---|---|
| Ab Name | (N) | (1/Ms, ×10⁵) | (1/s, ×10⁻²) | (M, ×10⁻⁸) | (RU) | (Min~Max) |
| 1E4(V1_VL1) | 2 | No-binding | — | — | — | — |
| ch9E4 | 2 | 5.097 ± | 1.594 ± | 3.262 ± | 80.27 ± 3.49 | 0.076~0.325 |
| | | 2.343 | 0.465 | 0.587 | | |
| hIgG1 (SSR) | 2 | No-binding | — | — | — | — |

TABLE 18
Comparison of Ch9E4 and Bispecific Hu1E4 Antibodies

| | Run | kₐ | k_{d} | K_{D} | Rₘₐₓ | Chi² |
|---|---|---|---|---|---|---|
| BsAb Name | (N) | (1/Ms, ×10⁵) | (1/s, ×10⁻²) | (M, ×10⁻⁸) | (RU) | (Min~Max) |
| 1E4(Ver.1_VL1) × | 2 | No-binding | — | — | — | — |
| Grabody B | | | | | | |
| ch9E4 | 2 | 5.536 ± | 1.875 ± | 3.460 ± | 83.14 ± 4.09 | 0.038~0.717 |
| | | 1.114 | 0.016 | 0.726 | | |
| hIgG1 (SSR) | 2 | No-binding | — | — | — | — |

TABLE 19
Comparison of Ch9E4 and Monospecific Hu1E4(V8_VL4) Antibodies

| | Run | kₐ | k_{d} | K_{D} | Rₘₐₓ | Chi² |
|---|---|---|---|---|---|---|
| Ab Name | (N) | (1/Ms, ×10⁵) | (1/s, ×10⁻²) | (M, ×10⁻⁸) | (RU) | (Min~Max) |
| 1E4(V8_VL4) | 2 | No-binding | — | — | — | — |
| ch9E4 | 2 | 6.713 ± | 1.857 ± | 2.777 ± | 77.01 ± 2.69 | 0.112~0.286 |
| | | 0.713 | 0.045 | 0.229 | | |
| hIgG1 (SSR) | 2 | No-binding | — | — | — | — |

These results demonstrate that the anti-α-syn antibodies of the present disclosure bind preferentially to α-syn aggregates and have no detectable binding affinity for α-syn monomers.

### Example 8: Evaluation of Antibody Yields

Expression constructs encoding mouse, chimeric, and humanized 1E4 antibodies were used to transfect host cells as described above. Antibodies produced in the cells were isolated and purified using MabSelect™ SuRe™ (MSS) resin (GE Healthcare). Antibody yields were measured by using a Thermo Scientific™ NanoDrop™ spectrophotometer. First, 1X PBS was loaded to confirm the zero point. Then, a predetermined amount of the purified antibody sample was loaded, and the protein quantity of each test sample was measured at a UV wavelength of 280 nm and was calculated by multiplying the protein concentration determined by NanoDrop™ with the total volume of the test sample. The purity of the purified antibody sample was analyzed by high performance liquid chromatography using an HLC-001 (Agilent 1200 series) system, and a ratio of the main peaks was quantified.

Table 20 below shows the productivity values (in mass of elute/harvested cell culture fluid (HCCF) volume of select antibodies.

TABLE 20
Productivity of Selected Antibodies

| | | | MSS eluate (mg)/ |
|---|---|---|---|
| | | | HCCF vol. (L) |
| Antibodies | Format | Origin | (mg/L) |
| Mouse 1E4 | Monospecific | Hybridoma | 16.2 |
| | antibody | | |
| ch1E4 | Monospecific | CHO | 200.0 |
| | antibody | | |
| ch1E4 | Monospecific | CHO | 521.9 |
| | antibody | | |
| ch1E4 × | Bispecific antibody | CHO | 75.1 |
| Grabody B | | | |
| ch1E4 × | Bispecific antibody | CHO | 93.1 |
| Grabody B | | | |
| hu1E4(V7_VL2) × | Bispecific antibody | CHO | 192.0 |
| Grabody B | | | |
| hu1E4(V5_VL5) × | Bispecific antibody | CHO | 164.0 |
| Grabody B | | | |

The above data show that the yields of the chimeric 1E4 antibody, humanized 1E4 antibodies, and bispecific antibodies thereof were markedly higher than the yield of the mouse 1E4 antibody.

To compare the productivities between the chimeric and humanized 1E4 monospecific antibody clones, 30 mL of a culture broth was purified through an MSS column, and the antibody amount in the purified product was analyzed using Nanodrop™. As shown in Table 21, all the humanized 1E4 monospecific antibody clones had superior productivity as compared to the chimeric 1E4 clone.

TABLE 21
Productivity of Select 1E4 Antibodies

| Antibody Clone Name | Productivity (mg) |
|---|---|
| Mouse 1E4 | N/A |
| ch1E4 | 0.611 or 0.358 |
| hu1E4(V1_VL1) | 2.33 |
| hu1E4(V2_VL2) | 2.16 |
| hu1E4(V3_VL2) | N/A |
| hu1E4(V4_VL2) | 1.942 |
| hu1E4(V7_VL2) | 2.267 |
| hu1E4(V8_VL4) | 2.05 |
| hu1E4(V9_VL4) | 2.23 |

### Example 9: Evaluation of Immunogenicity of Humanized Antibodies

The immunogenicity of hu1E4(V1_VL1) was evaluated by in silico analysis using Immune Epitope Database (IEDB) T-cell epitope prediction software, and compared to the immunogenicity of ch1E4.

Individual peptides with 15 residues spanning the entire antibody sequence of the Fv region were scored based on their ability to bind to MHC II complexes. The selected MHC II alleles were 11 human HLA DRB1 alleles and 4 human HLA DRB3/4/5 alleles. Non-redundant peptides binding to 12-15 (score 8 to 10) or 6-11 (score 4 to 7) MHC II molecules were marked as promiscuous high and promiscuous moderate, respectively, indicating the possibility to be potential T-cell epitopes

The data in Table 22 below show that the number of non-redundant peptides binding to 6-11 MHC II molecules (marked as promiscuous moderate) in the light chain are three (3) in ch1E4, while zero in hu1E4 VL1. The higher number of non-redundant peptides binding to MHC II molecules can be interpreted as the possibility of ch1E4 having higher immunogenicity than hu1E4.

TABLE 22
Immunogenicity of Select 1E4 Heavy and Light Chains

| | Promiscuous Moderate | Promiscuous High |
|---|---|---|
| | (Number of | (Number of |
| Chain | non-redundant peptides) | non-redundant peptides) |
| ch1E4_HC | 2 | 0 |
| ch1E4_LC | 3 | 1 |
| hu1E4(V1) HC | 2 | 0 |
| hu1E4(VL1) LC | 0 | 1 |

### Example 10: Hu1E4 Antibodies Show Superior Efficacy in Promoting Phagocytosis of α-Syn PFF

ch1E4 and hu1E4 antibodies (either in a monospecific format or a bispecific format with Grabody B) were evaluated for their microglial phagocytosis-promoting activity or monocyte phagocytosis-promoting activity. Comparisons were made among ch1E4, hu1E4, the existing anti-α-syn antibody ch9E4, the previous humanized anti-α-syn antibody 11F11(ver.2), and an hIgG1 control.

BV-2 cells (a microglia cell line) were grown in RPMI1640 supplemented with 10% fetal bovine serum and added to 96 well plates at 2.5×10⁵ cells/well. In a separate tube, α-syn PFF and the test antibody at various concentrations were mixed and incubated for 20 min. The concentrations of the antibody are shown in Table 23.

TABLE 23
Antibody Concentrations Used in α-Syn PFF Phagocytosis Assays
Antibody Concentrations (μg/mL) (nM)

| | |
|---|---|
| 0.03125 | (0.208125) |
| 0.0625 | (0.41625) |
| 0.125 | (0.8325) |
| 0.25 | (1.665) |
| 0.5 | (3.33) |
| 1 | (6.66) |
| 2 | (13.32) |
| 4 | (26.64) |

The mixture was added to the BV-2 cells and incubated at 37° C. for 20 minutes. After incubation, the plates were centrifuged and the supernatant was discarded. The cells were washed with PBS (adjusted to pH 2.5), resuspended and centrifuged, and then the supernatant was discarded to eliminate non-internalized PFF-Ab complex at the cell surface. This washing and centrifugation were repeated twice.

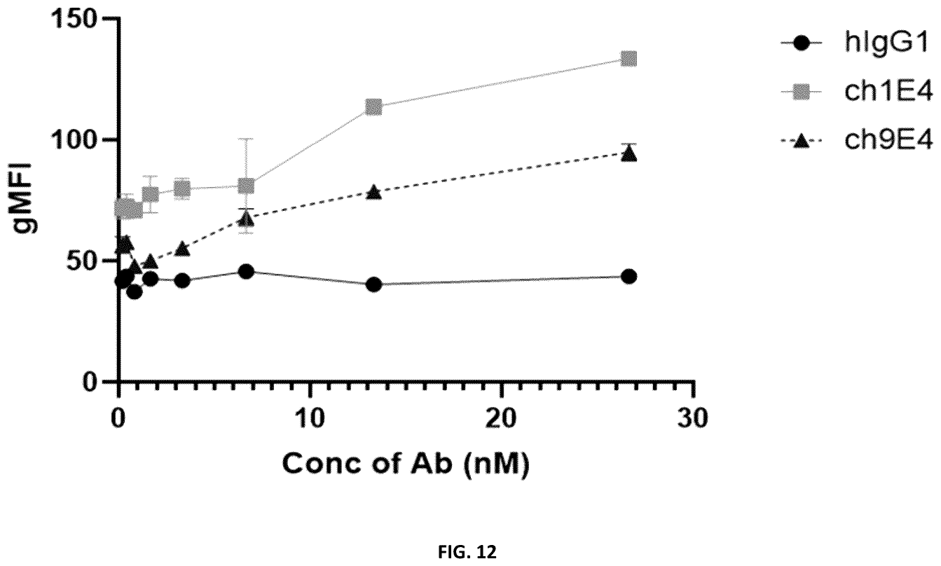

The cells were fixed with 4% paraformaldehyde for 30 minutes, and then washed with PBS. Then, 100 μL of 0.5% Triton® X-100 solution was added to the cells, which were further incubated for 20 min. The cells were washed with 1X PBS (pH 7.4) twice, and then 100 μL of 1% bovine serum albumin in PBS was added for blocking. The cells were washed with 1X PBS (pH 7.4) twice, and then incubated with 100 μL of the primary antibody (anti-α-syn, Santacruz, Cat: SC-10717, 1:1000) at room temperature for 1 hr. Then, 100 μL of PBS (with 0.05% Tween™ 20) was added, and the solution was resuspended and centrifuged (2000 rpm, 3 min, 4° C.); the supernatant was discarded. The above washing step was repeated before the secondary Ab (anti-rabbit-alexa488, Cell Signal, Cat: 4412S, 1:1000) was added to the cells and incubated in the dark for 1 hr. Again, 100 μL of PBS (with 0.05% Tween™ 20) was added, and the solution was resuspended and centrifuged (2000 rpm, 3 min, 4° C.). The supernatant was discarded, and the washing step was repeated. The cells were then resuspended with PBS and underwent FACS analysis, the results of which are quantified as the geometric mean fluorescence intensity (gMFI) across the tested antibody concentrations, and are shown in FIG. 12.

The data in FIG. 12 show that in BV-2 cells, ch 1E4 promoted phagocytosis of α-syn PFF to a much greater extent than the hIgG1 control and at a two-fold higher rate than ch9E4 (Roche/Prothena). These results indicate that ch 1E4 facilitates microglial removal of α-syn aggregates, which have been associated with neurotoxicity, inflammation, and neurodegeneration in neurons, as well as the etiology of Parkinson's disease. Hence, these results indicate that ch1E4 has stronger therapeutic potential than ch9E4.

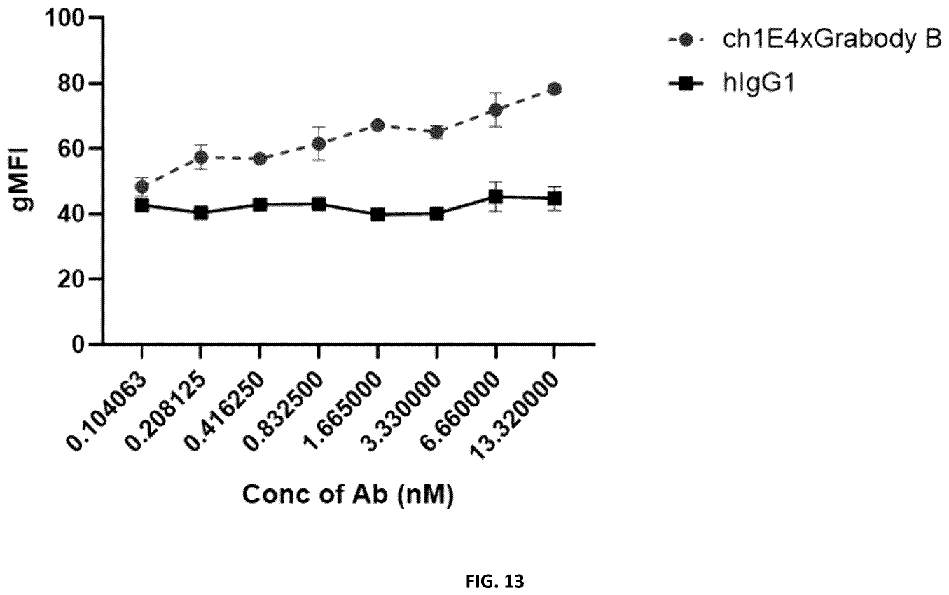

Next, monocyte phagocytosis-promoting activity of the ch 1E4×Grabody B antibody was compared with that of hIgG1. The assay was performed as described above, except that THP-1 cells were used instead of BV-2 cells. THP-1 is a human monocyte leukemia cell with a phagocytic function. The data in FIG. 13 show that in THP-1 cells, ch1E4×Grabody B promoted phagocytosis of α-syn PFF to a greater extent than the hIgG1 control. Considering that THP-1 cells express cell surface markers similar to those on microglia and phagocytose in a similar manner, these results suggest that the ch 1E4 and ch1E4×Grabody B BsAb both promote phagocytosis of extracellular α-syn PFF by human microglia.

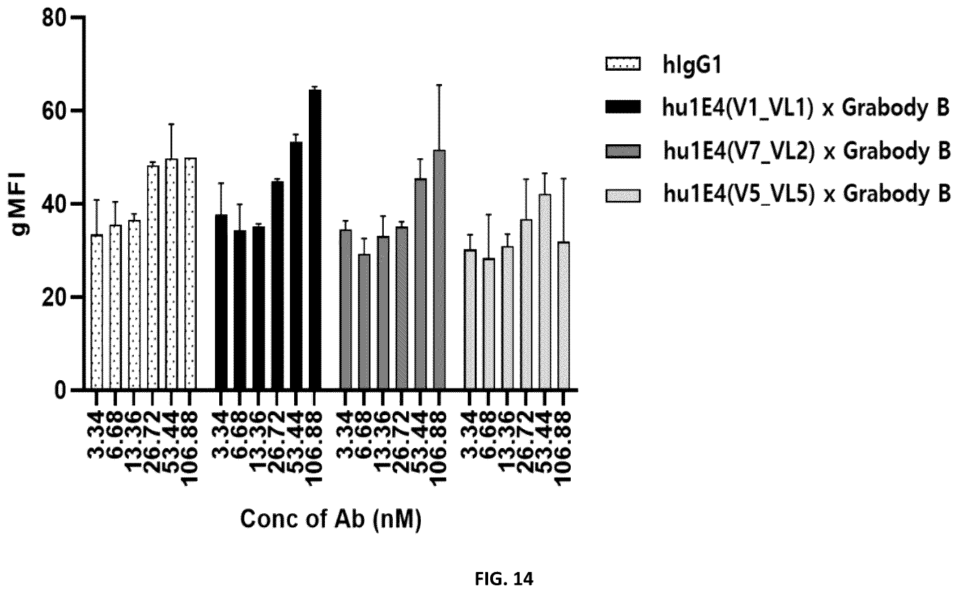

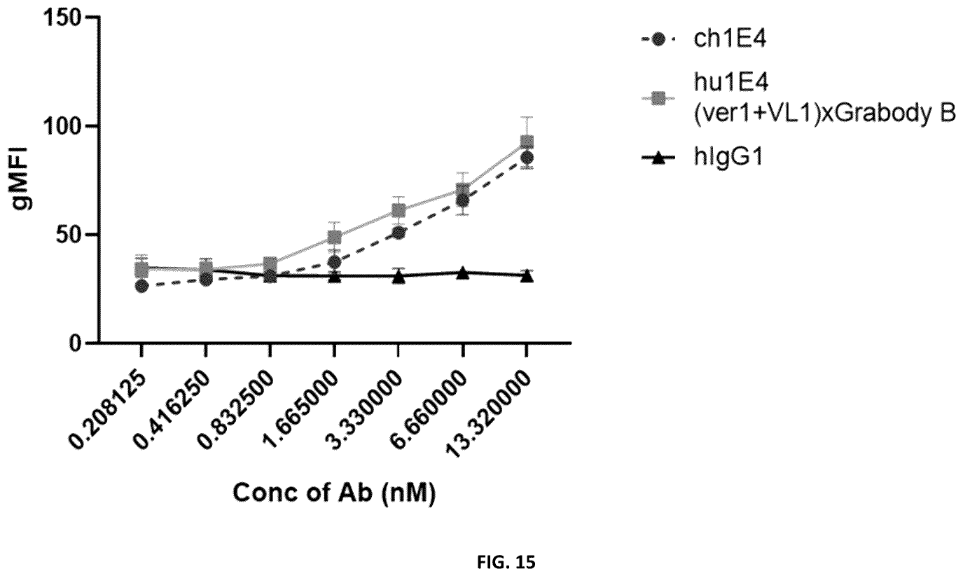

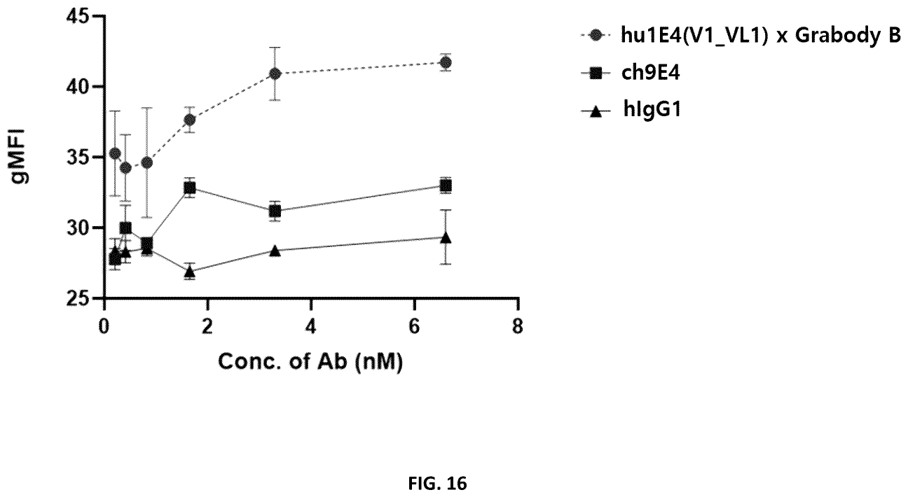

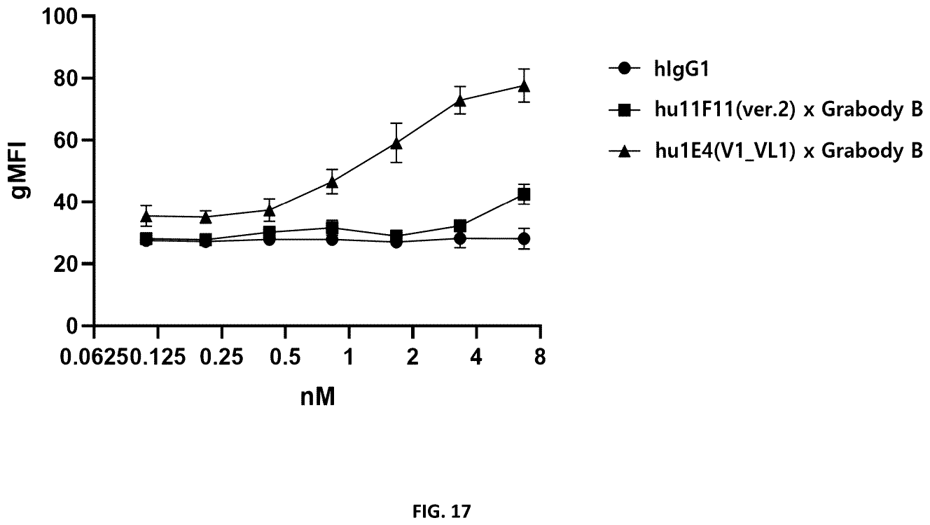

Additional hu1E4 BsAbs were compared for their activities in promoting microglial phagocytosis. The data in FIG. 14 show that among the tested BsAbs, hu1E4(V1_VL1)×Grabody B exhibited the highest activity in promoting phagocytosis. FIG. 15 compares the phagocytosis-promoting activity of ch1E4 and hu1E4(V1_VL1)×Grabody B. hu1E4(V1_VL1) showed phagocytosis-promoting activity as high as ch1E4, even slightly higher, despite its combination with Grabody B. FIG. 16 compares the phagocytosis-promoting activity of hu1E4(V1_VL1)×Grabody B and ch9E4. FIG. 17 compares the phagocytosis-promoting activity of hu1E4(V1_VL1)×Grabody B and hu11F11(ver.2)×Grabody B. The data in these figures demonstrate that hu1E4(V1_VL1)×Grabody B exhibited superior phagocytosis-promoting activity compared to hIgG1, ch9E4, hu11F11(ver.2), and ch1E4. Thus, the higher PFF-binding affinity of hu1E4(V1_VL1) correlates with a greater therapeutic potential.

### Example 11: Ch1E4 Shows Superior Ability to Bind Human α-Syn in mThy-1 Mice

Ch1E4 and other α-syn antibodies were tested for their ability to recognize α-syn aggregates in the mThy-1 mouse (UC San Diego), a transgenic mouse model that overexpresses human α-syn.

An 11-month-old mThy-1 female mouse was perfused intracardially with 1X PBS. Thereafter, the brain was fixed by immersion in a 4% PFA/1X PBS solution. After 12 hours of fixation, the brain was transferred to a 30% sucrose/1X PBS solution and stored for 3 days. The fixed brain was then sectioned into 40 μm thick pieces using a cryostat microtome. The sections were washed twice with 1X PBS and blocked with 3% hydrogen peroxide. All of the anti-α-syn antibodies used as the primary antibodies were diluted to 1 mg/mL with 1X PBS, added to the blocked sections at a ratio of 1:1000, and then incubated at 4° C. for 12 hours. After the blocked sections were sufficiently washed with 1X PBS, an anti-human IgG antibody was added at a ratio of 1:200, and the blocked sections were stained by 3, 3′-diaminobenzidine (DAB). The stained sections were imaged and analyzed by using a bright-field microscope.

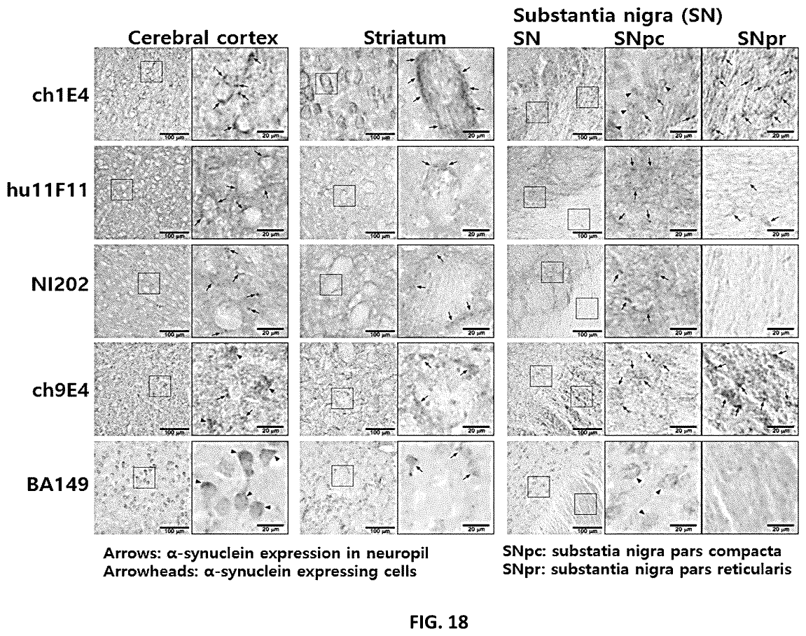

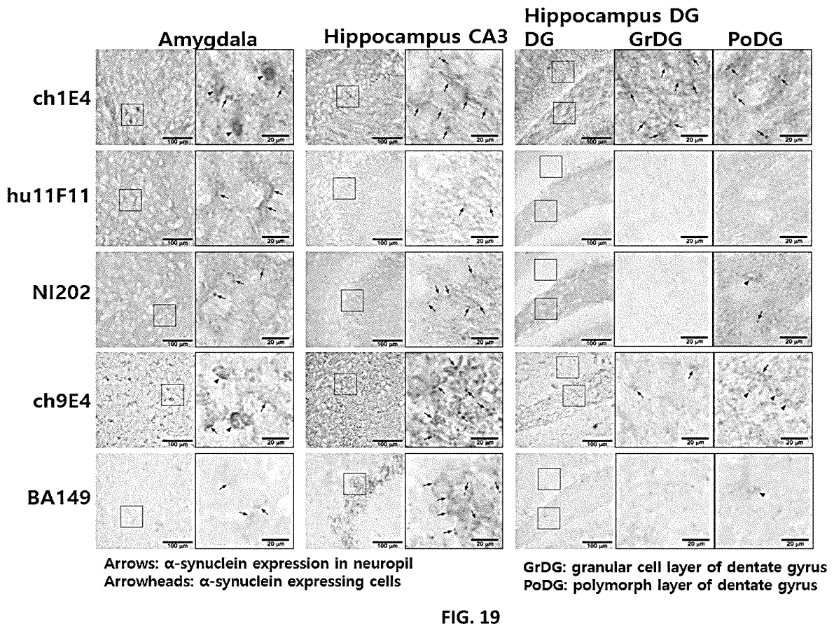

As shown in FIG. 18 and FIG. 19, ch1E4 recognized neuropil and α-syn aggregates in all parts of the analyzed brain. The commercially available antibodies (e.g., NI202 and BA149) exhibited very weak recognition of the α-syn aggregates. Ch9E4 exhibited recognition ability, but to a poor degree in the amygdala and the hippocampus, and showed an irregular and dirty staining pattern. BA149 did not recognize neuropil α-syn aggregates well and exhibited recognition ability only in some areas of the brain.

### Example 12: Staining of Postmortem Human Brain with Parkinson's Disease Using Ch1E4

To verify that 1E4 recognizes aggregated α-syn in the brain tissue of patients with synucleinopathies, ch 1E4 was used to stain the postmortem brain tissue of a 74-year-old male patient diagnosed with Parkinson's with dementia 4.5 years prior to death. A paraffin-embedded brain section from the deceased patient was further sectioned into 4 μm thick pieces. These brain sections were mounted onto slides, deparaffinized with xylene, and then rehydrated with a graded ethanol solution. After antigen retrieval, the brain sections were quenched by using 3% hydrogen peroxide with 0.005% Triton® X-100 in PBS. The brain sections were incubated with the primary antibody—the commercially available antibody Syn303 (BioLegend, Cat#: 824301) against phosphorylated α-syn, or ch1E4—at 4° C. for 12 hours. After the sections were washed with 1X PBS, a DAB reaction was carried out according to the manufacturer's manual. The stained sections were put on slides and their images were taken by analyzed by using a bright-field microscope.

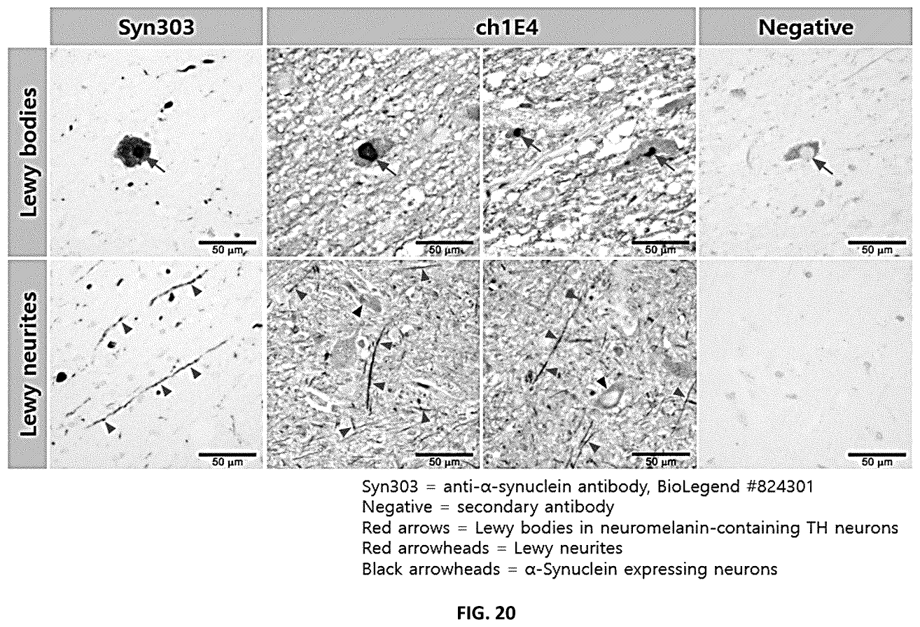

Phosphorylated α-syn is known to be major component of Lewy bodies and Lewy neurites, which contribute to neurodegeneration and brain dysfunction in Parkinson's disease. As shown in FIG. 20, ch1E4 recognized phosphorylated α-syn with similar sensitivity to Syn303, which was used in adjacent sections. The recognized pattern of phosphorylated α-syn was similar to those of Lewy bodies and Lewy neurites reported in the literature. These results demonstrate that ch1E4 recognized Lewy bodies and Lewy neurites in the brain tissue of the Parkinson's patient.

### Example 13: Brain Exposure to Ch1E4 Antibodies

The exposure to ch 1E4 and ch1E4×Grabody B in mThy-1 transgenic mice was analyzed. Each of the monospecific or bispecific ch 1E4 antibodies was intraperitoneally administered at 50 mg/kg once every three days for 11 days (at 0, 72, 144, and 216 hours). Seven-month-old male mThy-1 mice were divided into a monospecific antibody test group (n=3) and a bispecific antibody test group (n=2). Plasma was collected at 0, 72, and 264 hours. The animals were anesthetized with chloral hydrate at 264 hours according to the humane regulations for pathological analysis of the brain. Then, the animals were intracardially perfused with 0.9% physiological saline.

Next, one half of the perfused brain (sagittal section) was stored in 4% paraformaldehyde (pH 7.4, 4° C.) in a phosphate buffer until analysis. The other half of the brain was immediately stored in a frozen state (−70° C.).

The pathological analysis was carried out as follows. The half of the brain fixed in paraformaldehyde was cut into serial sections with 40 μm thickness in a free-floating manner using a vibratome. To determine the expression level of phosphorylated α-syn in the brain in each animal group, a section including the cortex, the hippocampus, and the striatum was incubated overnight at 4° C. with a phosphorylated α-syn antibody. The markers used for aggregated α-syn included a phosphorylated-129 α-syn antibody (Abcam, #ab59264) or a full-length α-syn antibody (Cell Signaling Technology, #2642). The markers were then stained using DAB, according to the manufacturer's manual. The immunostained tissue sections were imaged under a bright-field microscope.

In the adjacent sections, the level of human IgG was analyzed through immunofluorescence staining. The sections of various brain areas were reacted at 4° C. for 12 hours with an anti-human IgG antibody, to which the fluorescent dye Alexa488 was conjugated. The brain sections were then washed with 1X PBS. To localize neurons, the sections were treated with an antibody against the neuronal marker NeuN (Chemicon, #MAB377). The sections were analyzed under a confocal microscope to measure the optical density of the cells.

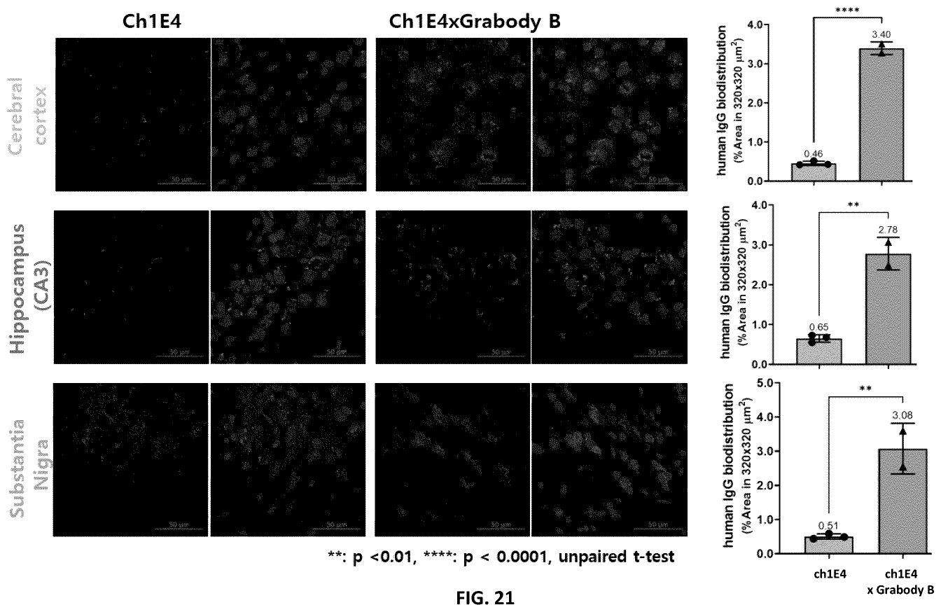

The data in FIG. 21 show that the ch1E4 BsAb had a higher level of exposure in the brain, compared to the monospecific ch1E4. Specifically, the bispecific antibody had a level of exposure 7.39-fold, 4.28-fold, and 6.04-fold higher in the cortex, the hippocampus, and the substantia nigra, respectively, than the monospecific antibody. The results indicate that Grabody B significantly improves blood-brain barrier (BBB) crossing ability of 1E4 in various brain areas.

### Example 14: Reduction of Phosphorylated α-Syn in vivo

In this Example, the experiment was performed in a manner similar to that of Example 13, except that the cortex, the amygdala, and the hippocampus in the mouse brain tissue were stained with an antibody against p-129 α-Syn after the test 1E4 antibodies were administered into the mice. P-129 α-syn is a form of α-syn in which the serine residue at position 129 is phosphorylated and has been implicated in the pathogenesis of Parkinson's disease. In the present experiment, this form of α-syn was observed in the form of dark brown dots or aggregates in the tissue stained with the anti-p-129 α-Syn antibody.

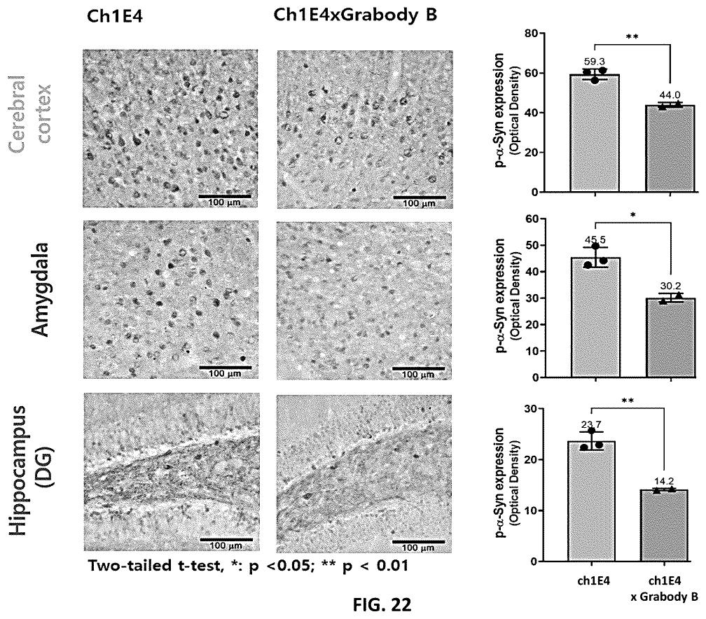

The data in FIG. 22 show the ability of both monospecific and bispecific ch1E4 to remove α-syn aggregates in vivo. The data also show that bispecific ch1E4 with Grabody B had an increased ability to reduce p-129 α-syn in the brain, including in the cerebral cortex, hippocampus, and amygdala, compared to monospecific ch1E4. Specifically, the bispecific antibody had the superior effect of reducing p-129 α-syn by 26%, 34%, and 40% in the cortex, amygdala, and hippocampus, respectively. Therefore, compared to the monospecific format, ch 1E4×Grabody B reduced the levels of p-129 α-syn and its aggregates much more effectively in an animal model of Parkinson's disease. These results are consistent with those in Example 13 and confirm that a larger amount of 1E4 BsAb can reach the brain due to the improved BBB penetration ability as compared to monospecific anti-α-syn antibodies, making the bispecific antibody an improved therapeutic tool for treating synucleinopathies.

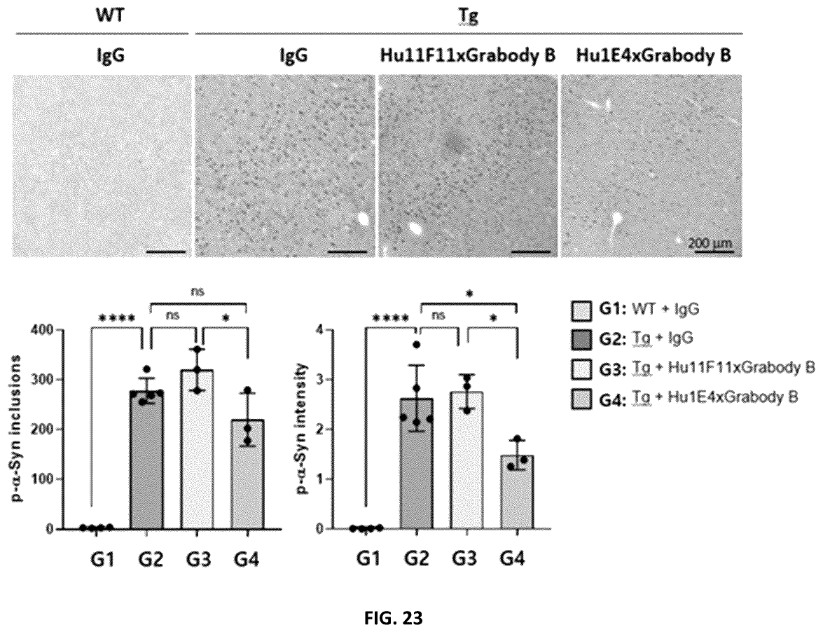

A similar experiment was performed with Grabody B-based bispecific humanized anti-α-syn antibodies. The data in FIG. 23 show that hu1E4(V1_VL1)×Grabody B reduced p-129 α-syn levels in mThy-1 mice to a greater degree than hu11F11×Grabody B, suggesting that hu1E4 is more effective in inhibiting α-syn transmission between neurons.

In summary, the results here demonstrate that bispecific hu1E4, such as hu1E4(V1_VL1)×Grabody B, is highly effective in removing p-129 α-syn from the brain in vivo.

### Example 15: Hu1E4 Reduces α-Syn Propagation in Dopaminergic Neurons

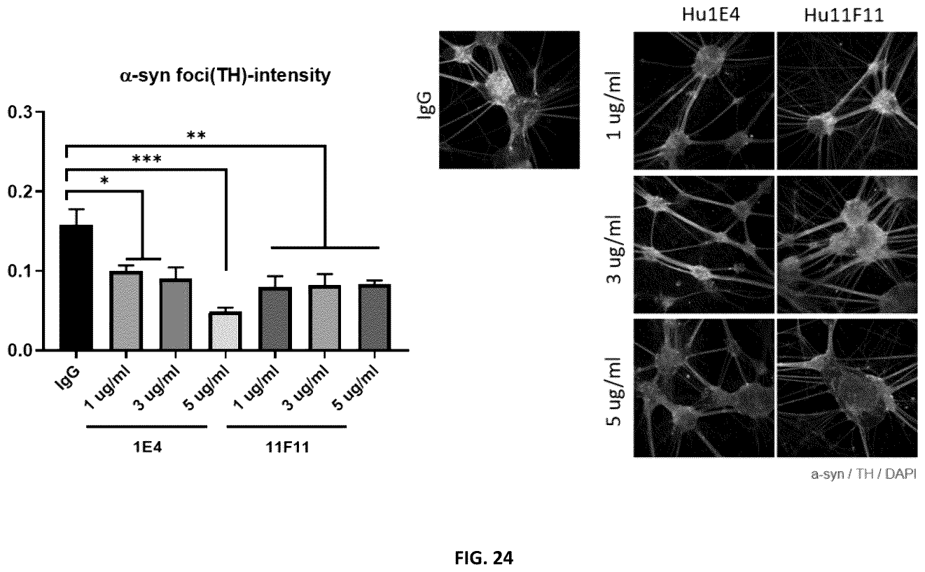

Hu1E4 (V1_VL1) and hu11F11 were tested for their ability to suppress α-syn propagation in dopaminergic neurons derived from induced pluripotent stem cells (iPSC) originating from Parkinson's disease patients. As shown in FIG. 24, at 5 μg/ml, hu1E4(V1_VL1) ameliorated α-syn propagation in these neurons to a larger extent than hu11F11. The intensity of colocalized α-syn staining with a dopaminergic neuronal cell marker, tyrosine hydroxylase, was significantly and dose-dependently reduced in the dopaminergic neurons after hu1E4 mAb treatment for 30 days. In the case of hu11F11 mAb, the reduction of colocalized intensity was less significant than hu1E4 mAb. These results suggest that hu1E4 is more effective in inhibiting α-syn transmission between neurons and therefore may be more effective for the treatment of Parkinson's disease and other alpha-synucleinopathies.

Unless otherwise defined herein, scientific and technical terms used in connection with the present disclosure shall have the meanings that are commonly understood by those of ordinary skill in the art. Exemplary methods and materials are described here, although methods and materials similar or equivalent to those described herein can also be used in the practice or testing of the present disclosure. All publications, patent applications, issued patents, and other documents referred to in this specification are herein incorporated by reference as if each individual publication, patent application, issued patent, or other document was specifically and individually indicated to be incorporated by reference in its entirety. Although a number of documents are cited herein, this citation does not constitute an admission that any of these documents forms part of the common general knowledge in the art. In case of conflict, the present specification, including definitions, will control. Further, unless otherwise required by context, singular terms shall include pluralities and plural terms shall include the singular. Throughout this specification and embodiments, the words “have” and “comprise,” or variations such as “has,” “having,” “comprises,” or “comprising,” will be understood to imply the inclusion of a stated integer or group of integers but not the exclusion of any other integer or group of integers. As used herein the term “about” refers to an amount that is near the stated amount by 10% or less. Further, headings provided herein are for convenience only and do not interpret the scope or meaning of the claimed embodiments.

### SEQUENCES

Amino acid and nucleotide (nt) sequences provided in the present disclosure are listed below (SEQ: SEQ ID NO). All sequences are amino acid sequences unless otherwise indicated by “nt.”

| Description | | SEQ | Sequences |
|---|---|---|---|
| VH | hu1E4 | 1 | QVQLVQSGAEVKKPGASVKVSCKASGYTFTNYLIEWVRQAPG |
| | (ver.1) | | QGLEWMGVINPGSGGTNYNEKFKGRVTMTRDTSISTAYMELS |
| | | | RLRSDDTAVYYCASGNYDTYWGQGTLVTVSS |
| | hu1E4 | 2 | QVQLVQSGAEVKKPGASVKVSCKASGYTFTNYLIEWVRQAPG |
| | (ver.2) | | QGLEWIGVINPGSGGTNYNEKFKGKVTLTADKSISTAYMELS |
| | | | RLRSDDTAVYYCASGNYDTYWGQGTLVTVSS |
| | hu1E4 | 3 | QVQLVQSGAEVKKPGASVKVSCKASGYAFTNYLIEWVKQAPG |
| | (ver.3) | | QGLEWIGVINPGSGGTNYNEKFKGKVTLTADKSISTAYMELS |
| | | | RLRSDDTAVYYCASGNYDTYWGQGTLVTVSS |
| | hu1E4 | 4 | QVQLVOSGAEVVKPGASVKVSCKASGYAFTNYLIEWVKQRPG |
| | (ver.4) | | QGLEWIGVINPGSGGTNYNEKFKGKATLTADKSISTAYMELS |
| | | | RLRSDDTAVYYCASGNYDTYWGQGTLVTVSS |
| | hu1E4 | 5 | QVQLVQSGAEVKKPGASVKVSCKASGYAFTNYLIEWVKQAPG |
| | (ver.5) | | QGLEWIGVINPGSGGTNYNEKFKGRATLTRDTSISTAYMELS |
| | | | RLRSDDTAVYFCASGNYDTYWGQGTLVTVSS |
| | hu1E4 | 6 | QVQLVQSGAEVKKPGASVKVSCKASGYAFTNYLIEWVKQAPG |
| | (ver.6) | | QGLEWIGVINPGSGGTNYNEKFKGRATLTADTSISTAYMELS |
| | | | RLRSDDTAVYFCASGNYDTYWGQGTLVTVSS |
| | hu1E4 | 7 | QVQLVQSGAEVKKPGASVKVSCKASGYAFTNYLIEWVKQAPG |
| | (ver.7) | | QGLEWMGVINPGSGGTNYNEKFKGKATLTADKSISTAYMELS |
| | | | RLRSDDTAVYYCASGNYDTYWGQGTLVTVSS |
| | hu1E4 | 8 | QVQLVQSGAEVKKPGASVKVSCKASGYTFTNYLIEWVRQAPG |
| | (ver.8) | | QGLEWMGVINPGSGGTNYNEKFKGRVTMTADKSISTAYMELS |
| | | | RLRSDDTAVYYCASGNYDTYWGQGTLVTVSS |
| | hu1E4 | 9 | QVQLVQSGAEVKKPGASVKVSCKASGYTFTNYLIEWVRQAPG |
| | (ver.9) | | QGLEWIGVINPGSGGTNYNEKFKGRATLTADKSISTAYMELS |
| | | | RLRSDDTAVYYCASGNYDTYWGQGTLVTVSS |
| | ch1E4_VH | 10 | QVQLQQSGAELVRPGTSVKVSCKASGYAFTNYLIEWVKQRPG |
| | | | QGLEWIGVINPGSGGTNYNEKFKGKATLTADKSSSTAYMQLS |
| | | | SLTSDDSAVYFCASGNYDTYWGQGTLVTVSA |
| VL | hu1E4 | 11 | DIVMTQTPLSLSVTPGQPASISCRSSQSLVHSNGNTYLHWYL |
| | (VL1) | | QKPGQSPQLLIYKVSNRFSGVPDRFSGSGSGTDFTLKISRVE |
| | | | AEDVGVYYCSQSTHVPRTFGQGTKLEIK |
| | hu1E4 | 12 | DVVMTQTPLSLSVTPGQPASISCRSSQSLVHSNGNTYLHWYL |
| | (VL2) | | QKPGQSPQLLIYKVSNRFSGVPDRFSGSGSGTDFTLKISRVE |
| | | | AEDVGVYYCSQSTHVPRTFGQGTKLEIK |
| | hu1E4 | 13 | DVVMTQSPLSLPVTLGQPASISCRSSQSLVHSNGNTYLHWFQ |
| | (VL3) | | QRPGQSPRRLIYKVSNRFSGVPDRFSGSGSGTDFTLKISRVE |
| | | | AEDVGVYYCSQSTHVPRTFGGGTKVEIK |
| | hu1E4 | 14 | DVVMTQSPLSLPVTLGQPASISCRSSQSLVHSNGNTYLHWYQ |
| | (VL4) | | QRPGQSPRLLIYKVSNRFSGVPDRFSGSGSGTDFTLKISRVE |
| | | | AEDVGVYYCSQSTHVPRTFGGGTKVEIK |
| | hu1E4 | 15 | DVVMTQSPLSLPVTLGQPASISCRSSQSLVHSNGNTYLHWYQ |
| | (VL5) | | QRPGQSPRLLIYKVSNRFSGVPDRFSGSGSGTDFTLKISRVE |
| | | | AEDVGVYFCSQSTHVPRTFGGGTKVEIK |
| | ch1E4_VL | 16 | DVVMTQTPLSLPVSLGDQASISCRSSQSLVHSNGNTYLHWYL |
| | | | QKPGQSPKLLIYKVSNRFSGVPDRFSGSGSGTDFTLKISRVE |
| | | | AEDLGVYFCSQSTHVPRTFGGGTKLEIK |
| VH | hu1E4 | 17 | CAGGTGCAACTGGTCCAATCTGGTGCCGAAGTGAAAAAGCCC |
| (nt) | (ver.1) | | GGTGCAAGTGTTAAAGTTTCCTGCAAAGCATCTGGCTATACT |
| | | | TTTACTAATTATCTGATAGAATGGGTACGACAAGCTCCAGGT |
| | | | CAAGGTCTTGAATGGATGGGCGTGATTAACCCAGGGAGTGGA |
| | | | GGAACTAATTACAATGAGAAGTTCAAAGGACGAGTGACAATG |
| | | | ACCCGTGATACCAGTATTAGCACCGCATACATGGAGTTGAGC |
| | | | AGGCTTCGTAGCGACGACACCGCAGTATATTATTGTGCTTCC |
| | | | GGCAATTATGATACCTATTGGGGGCAGGGGACTCTCGTAACC |
| | | | GTCTCCAGT |
| | hu1E4 | 18 | CAAGTTCAGCTTGTGCAAAGCGGAGCTGAGGTCAAAAAACCA |
| | (ver.2) | | GGAGCATCTGTGAAAGTGTCATGCAAAGCATCTGGTTACACT |
| | | | TTTACTAACTATCTCATTGAGTGGGTCAGACAGGCTCCAGGA |
| | | | CAAGGGTTGGAATGGATTGGGGTCATCAACCCTGGGTCAGGG |
| | | | GGGACAAATTACAATGAAAAATTTAAAGGGAAGGTGACACTC |
| | | | ACAGCAGATAAGTCTATTTCAACCGCTTATATGGAACTTTCC |
| | | | CGCCTTAGGAGCGATGATACAGCAGTCTATTATTGCGCTTCA |
| | | | GGGAACTACGACACTTACTGGGGGCAAGGCACACTCGTTACA |
| | | | GTGTCCTCC |
| | hu1E4 | 19 | CAAGTGCAGCTGGTGCAGTCTGGCGCAGAAGTGAAGAAACCT |
| | (ver.3) | | GGGGCTTCTGTGAAGGTCAGCTGTAAAGCTTCTGGGTATGCT |
| | | | TTTACAAACTATCTCATAGAATGGGTCAAACAAGCTCCAGGT |
| | | | CAGGGCCTGGAGTGGATCGGTGTAATCAACCCTGGTAGTGGC |
| | | | GGAACAAACTACAATGAGAAGTTCAAAGGCAAAGTGACTCTT |
| | | | ACCGCCGACAAAAGCATTTCTACAGCTTATATGGAGTTGTCA |
| | | | AGGTTGAGAAGCGACGACACAGCAGTTTACTATTGCGCTTCA |
| | | | GGAAATTATGACACATACTGGGGTCAGGGTACACTGGTGACT |
| | | | GTGTCCTCT |
| | hu1E4 | 20 | CAAGTCCAATTGGTCCAGAGTGGAGCTGAAGTTGTTAAACCA |
| | (ver.4) | | GGAGCAAGCGTTAAAGTTAGTTGTAAGGCATCAGGTTACGCC |
| | | | TTCACCAACTACTTGATTGAATGGGTAAAGCAACGTCCCGGT |
| | | | CAGGGCCTTGAGTGGATTGGGGTTATAAATCCTGGTTCTGGG |
| | | | GGAACTAATTACAACGAGAAGTTCAAGGGCAAGGCCACCCTT |
| | | | ACTGCCGACAAGTCCATAAGCACCGCCTATATGGAATTGAGC |
| | | | AGATTGCGAAGTGATGACACCGCCGTCTACTATTGTGCTTCA |
| | | | GGTAATTATGATACCTATTGGGGGCAAGGAACACTGGTGACC |
| | | | GTATCTTCC |
| | hu1E4 | 21 | CAAGTCCAACTGGTTCAGTCTGGAGCTGAAGTAAAAAAGCCT |
| | (ver.5) | | GGGGCCAGTGTGAAAGTATCCTGCAAAGCCAGCGGCTACGCA |
| | | | TTTACTAATTATCTGATTGAATGGGTCAAGCAGGCACCAGGA |
| | | | CAGGGTCTGGAATGGATTGGGGTTATAAATCCAGGAAGTGGG |
| | | | GGGACTAATTACAATGAGAAATTTAAAGGCCGAGCAACCTTG |
| | | | ACACGCGATACCTCAATAAGTACCGCATATATGGAACTCAGT |
| | | | CGTCTGCGAAGTGATGATACCGCTGTTTATTTCTGTGCTAGT |
| | | | GGCAACTATGACACATACTGGGGACAGGGTACATTGGTAACA |
| | | | GTGAGTAGC |
| | hu1E4 | 22 | CAAGTGCAGCTGGTACAGTCCGGAGCTGAAGTCAAGAAACCA |
| | (ver.6) | | GGGGCCTCAGTGAAAGTTTCATGTAAGGCTTCTGGATATGCC |
| | | | TTTACCAACTACCTTATCGAGTGGGTTAAACAGGCCCCAGGG |
| | | | CAAGGATTGGAGTGGATCGGGGTCATCAATCCAGGATCAGGT |
| | | | GGTACAAACTATAATGAGAAATTCAAGGGTAGAGCTACTCTG |
| | | | ACAGCCGATACAAGTATCAGCACTGCATATATGGAGTTGAGC |
| | | | CGTCTCCGCAGCGACGACACAGCCGTATATTTCTGTGCATCT |
| | | | GGCAATTATGACACATACTGGGGCCAGGGAACTCTTGTCACC |
| | | | GTCTCCTCA |
| | hu1E4 | 23 | CAGGTCCAATTGGTGCAATCAGGGGCCGAGGTCAAAAAACCT |
| | (ver.7) | | GGAGCAAGTGTTAAAGTGAGCTGTAAGGCCTCTGGCTACGCC |
| | | | TTTACAAATTATCTCATCGAATGGGTAAAGCAAGCACCAGGG |
| | | | CAAGGACTCGAATGGATGGGCGTGATTAACCCAGGATCAGGG |
| | | | GGAACCAATTACAACGAAAAGTTTAAGGGGAAGGCTACACTG |
| | | | ACTGCTGATAAATCCATATCTACTGCATACATGGAACTGAGC |
| | | | CGGCTCCGTTCTGACGACACTGCTGTCTATTATTGCGCTTCT |
| | | | GGCAATTATGATACTTATTGGGGGCAAGGCACTCTGGTTACA |
| | | | GTCAGCTCC |
| | hu1E4 | 24 | CAAGTCCAGCTCGTACAAAGTGGTGCCGAGGTTAAGAAACCT |
| | (ver.8) | | GGTGCTAGTGTGAAAGTCTCATGCAAGGCTAGTGGCTACACT |
| | | | TTTACCAATTACCTGATTGAATGGGTCCGACAAGCCCCAGGT |
| | | | CAGGGATTGGAGTGGATGGGAGTCATTAACCCTGGCTCAGGT |
| | | | GGCACTAATTATAACGAAAAGTTTAAAGGCCGTGTGACTATG |
| | | | ACCGCCGACAAGAGCATCTCTACTGCATACATGGAACTCAGC |
| | | | CGTCTGCGTTCTGATGATACTGCTGTGTATTATTGTGCATCT |
| | | | GGGAACTACGACACTTATTGGGGACAGGGCACTCTTGTAACC |
| | | | GTATCCTCT |
| | hu1E4 | 25 | CAAGTGCAGTTGGTGCAAAGTGGCGCTGAAGTTAAGAAGCCT |
| | (ver.9) | | GGTGCTTCAGTCAAAGTATCTTGCAAAGCTAGTGGTTACACC |
| | | | TTTACCAACTACCTGATCGAATGGGTCAGGCAGGCACCCGGA |
| | | | CAAGGTTTGGAATGGATTGGAGTCATAAACCCCGGCTCCGGA |
| | | | GGAACTAACTACAACGAGAAATTCAAAGGCAGGGCCACTCTC |
| | | | ACCGCTGATAAAAGTATTTCAACTGCATATATGGAACTTTCT |
| | | | AGGCTTCGGTCAGATGACACTGCTGTCTACTATTGTGCATCC |
| | | | GGCAATTACGATACTTATTGGGGCCAGGGGACATTGGTCACA |
| | | | GTGTCTTCT |
| | ch1E4_VH | 26 | CAAGTCCAACTCCAACAAAGTGGAGCAGAGTTGGTGCGCCCA |
| | | | GGCACAAGTGTCAAAGTTAGCTGTAAGGCTTCAGGCTATGCA |
| | | | TTTACAAATTACTTGATTGAGTGGGTGAAACAGCGACCCGGA |
| | | | CAAGGCTTGGAATGGATCGGTGTCATCAATCCAGGATCTGGG |
| | | | GGAACAAATTATAACGAAAAATTCAAGGGGAAAGCCACACTC |
| | | | ACCGCCGATAAAAGTTCATCCACCGCATATATGCAGCTTAGC |
| | | | TCTCTCACATCTGACGATAGCGCAGTGTATTTCTGCGCATCC |
| | | | GGTAATTACGATACTTATTGGGGCCAAGGTACTCTGGTGACC |
| | | | GTTAGCGCC |
| VL | hu1E4 | 27 | GACATTGTGATGACTCAGACTCCCCTTAGTCTTAGTGTTACC |
| (nt) | (VL1) | | CCCGGGCAACCAGCATCAATATCATGCCGCAGCTCACAGAGT |
| | | | CTGGTTCACTCTAATGGCAACACTTATCTTCACTGGTATCTC |
| | | | CAGAAACCCGGTCAATCTCCACAACTGTTGATCTACAAAGTG |
| | | | TCCAATCGCTTTTCAGGCGTGCCCGACCGATTTAGTGGTAGC |
| | | | GGGTCAGGGACCGACTTTACTCTGAAAATTTCAAGAGTAGAG |
| | | | GCTGAAGATGTAGGAGTCTACTACTGCTCTCAAAGTACCCAT |
| | | | GTTCCCAGAACATTCGGCCAGGGCACCAAACTGGAGATTAAG |
| | hu1E4 | 28 | GATGTAGTGATGACACAAACCCCTTTGTCATTGAGCGTCACC |
| | (VL2) | | CCCGGACAGCCCGCTTCCATTAGTTGCCGAAGCTCACAGAGT |
| | | | CTCGTCCACTCCAACGGTAACACATACCTCCACTGGTACTTG |
| | | | CAGAAGCCTGGACAGAGCCCCCAATTGCTGATATATAAAGTG |
| | | | TCAAACAGATTTAGCGGGGTGCCCGATAGATTCTCCGGTAGC |
| | | | GGCTCAGGCACCGACTTTACATTGAAGATCAGTCGCGTCGAA |
| | | | GCAGAGGATGTTGGCGTGTATTATTGCAGTCAAAGCACACAT |
| | | | GTTCCTAGGACTTTTGGGCAGGGTACTAAGTTGGAAATAAAG |
| | hu1E4 | 29 | GATGTAGTAATGACTCAATCTCCACTTAGCCTCCCCGTGACA |
| | (VL3) | | CTCGGCCAACCAGCAAGCATATCCTGTCGTTCCAGCCAGTCC |
| | | | CTCGTGCATTCTAACGGCAACACCTACTTGCATTGGTTCCAA |
| | | | CAGCGTCCAGGACAGAGTCCTCGGCGTTTGATATATAAGGTG |
| | | | TCCAACCGATTCAGCGGGGTTCCTGATAGGTTTTCTGGATCT |
| | | | GGTTCAGGCACCGATTTCACTTTGAAAATATCTCGCGTTGAG |
| | | | GCCGAAGACGTAGGTGTGTATTACTGTTCCCAGTCCACCCAT |
| | | | GTGCCTCGAACCTTCGGGGGAGGTACAAAAGTGGAAATCAAA |
| | hu1E4 | 30 | GATGTGGTAATGACACAGTCCCCTCTCTCATTGCCCGTGACA |
| | (VL4) | | CTGGGGCAGCCCGCTTCCATTAGCTGTCGAAGTAGTCAGAGC |
| | | | TTGGTTCATAGCAATGGTAATACATATTTGCACTGGTATCAA |
| | | | CAACGTCCAGGCCAATCACCACGACTCCTTATTTACAAAGTC |
| | | | TCTAACCGGTTTAGCGGTGTACCAGACCGTTTCTCTGGTTCA |
| | | | GGGAGTGGGACCGATTTCACTCTTAAAATTAGTCGGGTCGAA |
| | | | GCCGAAGACGTTGGCGTCTACTATTGCAGCCAGTCAACTCAT |
| | | | GTACCACGAACTTTCGGCGGCGGTACCAAGGTTGAGATTAAG |
| | hu1E4 | 31 | GATGTTGTGATGACACAGTCCCCACTCTCTCTGCCCGTCACA |
| | (VL5) | | TTGGGTCAACCCGCAAGCATCTCCTGTCGTTCCAGCCAGAGT |
| | | | CTTGTACATTCCAATGGCAATACTTACCTCCATTGGTACCAA |
| | | | CAGAGACCCGGTCAAAGCCCTCGTCTCTTGATATATAAGGTT |
| | | | TCAAACAGGTTCTCAGGGGTACCCGACCGATTCAGCGGTTCA |
| | | | GGGTCCGGGACAGACTTCACCCTCAAAATTAGTCGTGTGGAG |
| | | | GCTGAGGACGTGGGTGTATATTTCTGTAGCCAGAGTACCCAT |
| | | | GTACCTCGCACCTTTGGGGGAGGCACCAAGGTTGAAATTAAA |
| | ch1E4_VL | 32 | GACGTTGTAATGACTCAAACACCACTTTCTTTGCCAGTATCA |
| | | | CTTGGAGATCAAGCTTCTATCTCATGTAGGAGCTCCCAGTCA |
| | | | CTGGTGCATTCAAACGGAAACACATACCTTCACTGGTATCTG |
| | | | CAAAAACCAGGCCAATCACCAAAGCTTTTGATATACAAAGTG |
| | | | TCAAATCGTTTTAGCGGAGTACCAGACCGATTCAGCGGGAGC |
| | | | GGAAGCGGGACTGACTTCACACTTAAGATTAGCAGAGTGGAA |
| | | | GCCGAAGACCTGGGTGTCTATTTTTGCAGCCAGAGCACCCAT |
| | | | GTCCCCCGCACATTCGGAGGTGGAACCAAGCTGGAGATAAAA |
| 1E4 | HCDR1 | 33 | NYLIE |
| | HCDR2 | 34 | VINPGSGGTNYNEKFKG |
| | HCDR3 | 35 | GNYDTY |
| 1E4 | LCDR1 | 36 | RSSQSLVHSNGNTYLH |
| | LCDR2 | 37 | KVSNRFS |
| | LCDR3 | 38 | SQSTHVPRT |
| hu1E4 hIgG1 | | 39 | ASTKGPSVFPLAPSSKSTSGGTAALGCLVKDYFPEPVTVSWN |
| heavy chain | | | SGALTSGVHTFPAVLQSSGLYSLSSVVTVPSSSLGTQTYICN |
| constant region | | | VNHKPSNTKVDKKVEPKSCDKTHTCPPCPAPELLGGPSVFLF |
| (knob) | | | PPKPKDTLMISRTPEVTCVVVDVSHEDPEVKFNWYVDGVEVH |
| | | | NAKTKPREEQYNSTYRVVSVLTVLHQDWLNGKEYKCKVSNKA |
| | | | LPAPIEKTISKAKGQPREPQVYTLPPSRDELTKNQVSLWCLV |
| | | | KGFYPSDIAVEWESNGQPENNYKTTPPVLDSDGSFFLYSKLT |
| | | | VDKSRWQQGNVFSCSVMHEALHNHYTQKSLSLSPGK |
| hu1E4 hIgG1 | | 40 | ASTKGPSVFPLAPSSKSTSGGTAALGCLVKDYFPEPVTVSWN |
| heavy chain | | | SGALTSGVHTFPAVLQSSGLYSLSSVVTVPSSSLGTQTYICN |
| constant region | | | VNHKPSNTKVDKKVEPKSCDKTHTCPPCPAPELLGGPSVFLF |
| (hole) | | | PPKPKDTLMISRTPEVTCVVVDVSHEDPEVKFNWYVDGVEVH |
| | | | NAKTKPREEQYNSTYRVVSVLTVLHQDWLNGKEYKCKVSNKA |
| | | | LPAPIEKTISKAKGQPREPQVYTLPPSRDELTKNQVSLSCAV |
| | | | KGFYPSDIAVEWESNGQPENNYKTTPPVLDSDGSFFLVSKLT |
| | | | VDKSRWQQGNVFSCSVMHEALHNHYTQKSLSLSPGK |
| hu1E4 hIgG1 | | 41 | ASTKGPSVFPLAPSSKSTSGGTAALGCLVKDYFPEPVTVSWN |
| heavy chain | | | SGALTSGVHTFPAVLQSSGLYSLSSVVTVPSSSLGTQTYICN |
| constant region | | | VNHKPSNTKVDKKVEPKSCDKTHTCPPCPAPELLGGPSVFLF |
| (knob) (M428L) | | | PPKPKDTLMISRTPEVTCVVVDVSHEDPEVKFNWYVDGVEVH |
| | | | NAKTKPREEQYNSTYRWSVLTVLHQDWLNGKEYKCKVSNKA |
| | | | LPAPIEKTISKAKGQPREPQVYTLPPSRDELTKNQVSLWCLV |
| | | | KGFYPSDIAVEWESNGQPENNYKTTPPVLDSDGSFFLYSKLT |
| | | | VDKSRWQQGNVFSCSVLHEALHNHYTQKSLSLSPGK |
| hu1E4 hIgG1 | | 42 | ASTKGPSVFPLAPSSKSTSGGTAALGCLVKDYFPEPVTVSWN |
| heavy chain | | | SGALTSGVHTFPAVLQSSGLYSLSSVVTVPSSSLGTQTYICN |
| constant region | | | VNHKPSNTKVDKKVEPKSCDKTHTCPPCPAPELLGGPSVFLF |
| (hole) (M428L) | | | PPKPKDTLMISRTPEVTCVVVDVSHEDPEVKFNWYVDGVEVH |
| | | | NAKTKPREEQYNSTYRVVSVLTVLHQDWLNGKEYKCKVSNKA |
| | | | LPAPIEKTISKAKGQPREPQVYTLPPSRDELTKNQVSLSCAV |
| | | | KGFYPSDIAVEWESNGQPENNYKTTPPVLDSDGSFFLVSKLT |
| | | | VDKSRWQQGNVFSCSVLHEALHNHYTQKSLSLSPGK |
| hu1E4 light chain | | 43 | RTVAAPSVFIFPPSDEQLKSGTASVVCLLNNFYPREAKVQWK |
| (human kappa) | | | VDNALQSGNSQESVTEQDSKDSTYSLSSTLTLSKADYEKHKV |
| constant region | | | YACEVTHQGLSSPVTKSFNRGEC |
| (G₄S)₃ Linker | | 44 | GGGGSGGGGSGGGGS |
| Grabody B | VL | 45 | QSVLTQPPSASGTPGQRVTISCTGSSSNIGSNDVSWYQQLPG |
| (anti- | | | TAPKLLIYANVNRPSGVPDRFSGSKSGTSASLAISGLRSEDE |
| IGF1R | | | ADYYCGTWAGSLNAYVFGCGTKLTVL |
| scFv) | | | |
| | LCDR1 | 46 | TGSSSNIGSNDVS |
| | LCDR2 | 47 | ANVNRPS |
| | LCDR3 | 48 | GTWAGSLNAYV |
| | (G₄S)₄ | 49 | GGGGSGGGGSGGGGSGGGGS |
| | Linker | | |
| | VH | 50 | EVQLLESGGGLVQPGGSLRLSCAASGFTFSSYDMSWVRQAPG |
| | | | KCLEWVSAISYDNANTYYADSVKGRFTISRDNSKNTLYLQMN |
| | | | SLRAEDTAVYYCAKGVLTTLMNWFDYWGQGTLVTVSS |
| | HCDR1 | 51 | GFTFSSYDMS |
| | HCDR2 | 52 | AISYDNANTYYADSVKG |
| | HCDR3 | 53 | GVLTTLMNWFDY |
| | Full | 54 | QSVLTQPPSASGTPGQRVTISCTGSSSNIGSNDVSWYQQLPG |
| | scFv | | TAPKLLIYANVNRPSGVPDRFSGSKSGTSASLAISGLRSEDE |
| | sequence | | ADYYCGTWAGSLNAYVFGCGTKLTVLGGGGSGGGGSGGGGSG |
| | | | GGGSEVQLLESGGGLVQPGGSLRLSCAASGFTFSSYDMSWVR |
| | | | QAPGKCLEWVSAISYDNANTYYADSVKGRFTISRDNSKNTLY |
| | | | LQMNSLRAEDTAVYYCAKGVLTTLMNWFDYWGQGTLVTVSS |

| | | |
|---|---|---|
| hIgG1-Grabody B | 55 | ASTKGPSVFPLAPSSKSTSGGTAALGCLVKDYFPEPVTVSWN |
| constant region, | | SGALTSGVHTFPAVLQSSGLYSLSSVVTVPSSSLGTQTYICN |
| incl. Grabody B | | VNHKPSNTKVDKKVEPKSCDKTHTCPPCPAPELLGGPSVFLF |
| (hole) | | PPKPKDTLMISRTPEVTCVVVDVSHEDPEVKFNWYVDGVEVH |
| | | NAKTKPREEQYNSTYRVVSVLTVLHQDWLNGKEYKCKVSNKA |
| | | LPAPIEKTISKAKGQPREPQVYTLPPSRDELTKNQVSLSCAV |
| | | KGFYPSDIAVEWESNGQPENNYKTTPPVLDSDGSFFLVSKLT |
| | | VDKSRWQQGNVFSCSVMHEALHNHYTQKSLSLSPGKGGGGSG |
| | | GGGSGGGGSQSVLTQPPSASGTPGQRVTISCTGSSSNIGSND |
| | | VSWYQQLPGTAPKLLIYANVNRPSGVPDRFSGSKSGTSASLA |
| | | ISGLRSEDEADYYCGTWAGSLNAYVFGCGTKLTVLGGGGSGG |
| | | GGSGGGGSGGGGSEVQLLESGGGLVQPGGSLRLSCAASGFTF |
| | | SSYDMSWVRQAPGKCLEWVSAISYDNANTYYADSVKGRFTIS |
| | | RDNSKNTLYLQMNSLRAEDTAVYYCAKGVLTTLMNWFDYWGQ |
| | | GTLVTVSS |
| hIgG1-Grabody B | 39 | ASTKGPSVFPLAPSSKSTSGGTAALGCLVKDYFPEPVTVSWN |
| constant region | | SGALTSGVHTFPAVLQSSGLYSLSSVVTVPSSSLGTQTYICN |
| (knob) | | VNHKPSNTKVDKKVEPKSCDKTHTCPPCPAPELLGGPSVFLF |
| | | PPKPKDTLMISRTPEVTCVVVDVSHEDPEVKFNWYVDGVEVH |
| | | NAKTKPREEQYNSTYRVVSVLTVLHQDWLNGKEYKCKVSNKA |
| | | LPAPIEKTISKAKGQPREPQVYTLPPSRDELTKNQVSLWCLV |
| | | KGFYPSDIAVEWESNGQPENNYKTTPPVLDSDGSFFLYSKLT |
| | | VDKSRWQQGNVFSCSVMHEALHNHYTQKSLSLSPGK |
| hIgG1 (M428L)- | 56 | ASTKGPSVFPLAPSSKSTSGGTAALGCLVKDYFPEPVTVSWN |
| Grabody B | | SGALTSGVHTFPAVLQSSGLYSLSSVVTVPSSSLGTQTYICN |
| constant region, | | VNHKPSNTKVDKKVEPKSCDKTHTCPPCPAPELLGGPSVFLF |
| incl. Grabody B | | PPKPKDTLMISRTPEVTCVVVDVSHEDPEVKFNWYVDGVEVH |
| (hole) | | NAKTKPREEQYNSTYRVVSVLTVLHQDWLNGKEYKCKVSNKA |
| | | LPAPIEKTISKAKGQPREPQVYTLPPSRDELTKNQVSLSCAV |
| | | KGFYPSDIAVEWESNGQPENNYKTTPPVLDSDGSFFLVSKLT |
| | | VDKSRWQQGNVFSCSVLHEALHNHYTQKSLSLSPGKGGGGSG |
| | | GGGSGGGGSQSVLTQPPSASGTPGQRVTISCTGSSSNIGSND |
| | | VSWYQQLPGTAPKLLIYANVNRPSGVPDRFSGSKSGTSASLA |
| | | ISGLRSEDEADYYCGTWAGSLNAYVFGCGTKLTVLGGGGSGG |
| | | GGSGGGGSGGGGSEVQLLESGGGLVQPGGSLRLSCAASGFTF |
| | | SSYDMSWVRQAPGKCLEWVSAISYDNANTYYADSVKGRFTIS |
| | | RDNSKNTLYLQMNSLRAEDTAVYYCAKGVLTTLMNWFDYWGQ |
| | | GTLVTVSS |
| hIgG1 (M428L)- | 41 | ASTKGPSVFPLAPSSKSTSGGTAALGCLVKDYFPEPVTVSWN |
| Grabody B | | SGALTSGVHTFPAVLQSSGLYSLSSVVTVPSSSLGTQTYICN |
| contain region | | VNHKPSNTKVDKKVEPKSCDKTHTCPPCPAPELLGGPSVFLF |
| (knob) | | PPKPKDTLMISRTPEVTCVVVDVSHEDPEVKFNWYVDGVEVH |
| | | NAKTKPREEQYNSTYRVVSVLTVLHQDWLNGKEYKCKVSNKA |
| | | LPAPIEKTISKAKGQPREPQVYTLPPSRDELTKNQVSLWCLV |
| | | KGFYPSDIAVEWESNGQPENNYKTTPPVLDSDGSFFLYSKLT |
| | | VDKSRWQQGNVFSCSVLHEALHNHYTQKSLSLSPGK |
| hu1E4 (V1_VL1) | 57 | QVQLVQSGAEVKKPGASVKVSCKASGYTFTNYLIEWVRQAPG |
| hIgG1 (M428L) x | | QGLEWMGVINPGSGGTNYNEKFKGRVTMTRDTSISTAYMELS |
| Grabody B heavy | | RLRSDDTAVYYCASGNYDTYWGQGTLVTVSSASTKGPSVFPL |
| chain with | | APSSKSTSGGTAALGCLVKDYFPEPVTVSWNSGALTSGVHTF |
| Grabody B | | PAVLQSSGLYSLSSVVTVPSSSLGTQTYICNVNHKPSNTKVD |
| (hole) | | KKVEPKSCDKTHTCPPCPAPELLGGPSVFLFPPKPKDTLMIS |
| | | RTPEVTCVVVDVSHEDPEVKFNWYVDGVEVHNAKTKPREEQY |
| | | NSTYRVVSVLTVLHQDWLNGKEYKCKVSNKALPAPIEKTISK |
| | | AKGQPREPQVYTLPPSRDELTKNQVSLSCAVKGFYPSDIAVE |
| | | WESNGQPENNYKTTPPVLDSDGSFFLVSKLTVDKSRWQQGNV |
| | | FSCSVLHEALHNHYTQKSLSLSPGKGGGGSGGGGSGGGGSQS |
| | | VLTQPPSASGTPGQRVTISCTGSSSNIGSNDVSWYQQLPGTA |
| | | PKLLIYANVNRPSGVPDRFSGSKSGTSASLAISGLRSEDEAD |
| | | YYCGTWAGSLNAYVFGCGTKLTVLGGGGSGGGGSGGGGSGGG |
| | | GSEVQLLESGGGLVQPGGSLRLSCAASGFTFSSYDMSWVRQA |
| | | PGKCLEWVSAISYDNANTYYADSVKGRFTISRDNSKNTLYLQ |
| | | MNSLRAEDTAVYYCAKGVLTTLMNWFDYWGQGTLVTVSS |
| hu1E4 (V1_VL1) | 58 | QVQLVQSGAEVKKPGASVKVSCKASGYTFTNYLIEWVRQAPG |
| hIgG1 (M428L) x | | QGLEWMGVINPGSGGTNYNEKFKGRVTMTRDTSISTAYMELS |
| Grabody B heavy | | RLRSDDTAVYYCASGNYDTYWGQGTLVTVSSASTKGPSVFPL |
| chain without | | APSSKSTSGGTAALGCLVKDYFPEPVTVSWNSGALTSGVHTF |
| Grabody B (knob) | | PAVLQSSGLYSLSSVVTVPSSSLGTQTYICNVNHKPSNTKVD |
| | | KKVEPKSCDKTHTCPPCPAPELLGGPSVFLFPPKPKDTLMIS |
| | | RTPEVTCVVVDVSHEDPEVKFNWYVDGVEVHNAKTKPREEQY |
| | | NSTYRVVSVLTVLHQDWLNGKEYKCKVSNKALPAPIEKTISK |
| | | AKGQPREPQVYTLPPSRDELTKNQVSLWCLVKGFYPSDIAVE |
| | | WESNGQPENNYKTTPPVLDSDGSFFLYSKLTVDKSRWQQGNV |
| | | FSCSVLHEALHNHYTQKSLSLSPGK |
| hu1E4 (V1_VL1) | 59 | DIVMTQTPLSLSVTPGQPASISCRSSQSLVHSNGNTYLHWYL |
| Light Chain | | QKPGQSPQLLIYKVSNRFSGVPDRFSGSGSGTDFTLKISRVE |
| | | AEDVGVYYCSQSTHVPRTFGQGTKLEIKRTVAAPSVFIFPPS |
| | | DEQLKSGTASVVCLLNNFYPREAKVQWKVDNALQSGNSQESV |
| | | TEQDSKDSTYSLSSTLTLSKADYEKHKVYACEVTHQGLSSPV |
| | | TKSFNRGEC |
| Human alpha- | 60 | MDVFMKGLSKAKEGVVAAAEKTKQGVAEAAGKTKEGVLYVGS |
| synuclein | | KTKEGVVHGVATVAEKTKEQVTNVGGAVVTGVTAVAQKTVEG |
| | | AGSIAAATGFVKKDQLGKNEEGAPQEGILEDMPVDPDNEAYE |
| | | MPSEEGYQDYEPEA |
| Human | 61 | QVQLVQSGAEVKKPGASVKVSCKASGYTFT |
| VH1-02 FR1 | | |
| Human | 62 | WVRQAPGQGLEWMG |
| VH1-02 FR2 | | |
| Human | 63 | RVTMTRDTSISTAYMELSRLRSDDTAVYYCAR |
| VH1-02 FR3 | | |
| Hu1E4V1 | 64 | GYTFTNYL |
| HCDR1 (IMGT) | | |
| Hu1E4V1 | 65 | INPGSGGT |
| HCDR2 (IMGT) | | |
| Hu1E4V1 | 66 | ASGNYDTY |
| HCDR3 (IMGT) | | |
| Hu1E4VL1 | 67 | QSLVHSNGNTY |
| LCDR1 (IMGT) | | |
| Hu1E4VL1 | 68 | KVS |
| LCDR2 (IMGT) | | |
| Hu1E4VL1 | 69 | SQSTHVPRT |
| LCDR3 (IMGT) | | |
| Peptide linker | 70 | (GSGGS)ₙ |
| Peptide linker | 71 | (GGGGS)ₙ |
| Peptide linker | 72 | (GGGS)ₙ |
| Peptide linker | 73 | AAEPKSS |
| Peptide linker | 74 | AAEPKSSDKTHTCPPCP |
| Peptide linker | 75 | GGGG |
| Peptide linker | 76 | GGGGDKTHTCPPCP |
| Human IGF1R | 77 | MKSGSGGGSPTSLWGLLFLSAALSLWPTSGEICGPGIDIRND |
| | | YQQLKRLENCTVIEGYLHILLISKAEDYRSYRFPKLTVITEY |
| | | LLLFRVAGLESLGDLFPNLTVIRGWKLFYNYALVIFEMTNLK |
| | | DIGLYNLRNITRGAIRIEKNADLCYLSTVDWSLILDAVSNNY |
| | | IVGNKPPKECGDLCPGTMEEKPMCEKTTINNEYNYRCWTTNR |
| | | CQKMCPSTCGKRACTENNECCHPECLGSCSAPDNDTACVACR |
| | | HYYYAGVCVPACPPNTYRFEGWRCVDRDFCANILSAESSDSE |
| | | GFVIHDGECMQECPSGFIRNGSQSMYCIPCEGPCPKVCEEEK |
| | | KTKTIDSVTSAQMLQGCTIFKGNLLINIRRGNNIASELENFM |
| | | GLIEVVTGYVKIRHSHALVSLSFLKNLRLILGEEQLEGNYSF |
| | | YVLDNQNLQQLWDWDHRNLTIKAGKMYFAFNPKLCVSEIYRM |
| | | EEVTGTKGRQSKGDINTRNNGERASCESDVLHFTSTTTSKNR |
| | | IIITWHRYRPPDYRDLISFTVYYKEAPFKNVTEYDGQDACGS |
| | | NSWNMVDVDLPPNKDVEPGILLHGLKPWTQYAVYVKAVTLTM |
| | | VENDHIRGAKSEILYIRTNASVPSIPLDVLSASNSSSQLIVK |
| | | WNPPSLPNGNLSYYIVRWQRQPQDGYLYRHNYCSKDKIPIRK |
| | | YADGTIDIEEVTENPKTEVCGGEKGPCCACPKTEAEKQAEKE |
| | | EAEYRKVFENFLHNSIFVPRPERKRRDVMQVANTTMSSRSRN |
| | | TTAADTYNITDPEELETEYPFFESRVDNKERTVISNLRPFTL |
| | | YRIDIHSCNHEAEKLGCSASNFVFARTMPAEGADDIPGPVTW |
| | | EPRPENSIFLKWPEPENPNGLILMYEIKYGSQVEDQRECVSR |
| | | QEYRKYGGAKLNRLNPGNYTARIQATSLSGNGSWTDPVFFYV |
| | | QAKTGYENFIHLIIALPVAVLLIVGGLVIMLYVFHRKRNNSR |
| | | LGNGVLYASVNPEYFSAADVYVPDEWEVAREKITMSRELGQG |
| | | SFGMVYEGVAKGVVKDEPETRVAIKTVNEAASMRERIEFLNE |
| | | ASVMKEFNCHHVVRLLGVVSQGQPTLVIMELMTRGDLKSYLR |
| | | SLRPEMENNPVLAPPSLSKMIOMAGEIADGMAYLNANKFVHR |
| | | DLAARNCMVAEDFTVKIGDFGMTRDIYETDYYRKGGKGLLPV |
| | | RWMSPESLKDGVFTTYSDVWSFGVVLWEIATLAEQPYQGLSN |
| | | EQVLRFVMEGGLLDKPDNCPDMLFELMRMCWQYNPKMRPSFL |
| | | EIISSIKEEMEPGFREVSFYYSEENKLPEPEELDLEPENMES |
| | | VPLDPSASSSSLPLPDRHSGHKAENGPGPGVLVLRASFDERQ |
| | | PYAHMNGGRKNERALPLPQSSTC |

1. A method of treating an alpha-synucleinopathy in a human subject in need thereof, comprising administering a humanized antibody or antigen-binding fragment thereof that binds to human alpha-synuclein (α-syn), wherein the antibody or antigen-binding fragment comprises:
a heavy chain variable region (VH) comprising (i) heavy chain complementarity-determining regions 1-3 (HCDR1-3) set forth in SEQ ID NOs: 33-35, respectively, and (ii) heavy chain framework regions (FRs) 1, 2, and/or 3 from a human VH1-02 gene; and
a light chain variable region (VL) comprising light chain CDR1-3 (LCDR1-3) set forth in SEQ ID NOs: 36-38, respectively.

2. A method of treating an alpha-synucleinopathy in a human subject in need thereof, comprising administering a humanized antibody or antigen-binding fragment thereof that binds to human alpha-synuclein (α-syn), wherein the antibody or the antigen-binding fragment comprises:
a heavy chain variable region (VH) comprising heavy chain complementarity-determining regions 1-3 (HCDR1-3) set forth in SEQ ID NOs: 64-66, respectively; and
a light chain variable region (VL) comprising light chain CDR1-3 (LCDR1-3) set forth in SEQ ID NOs: 67-69, respectively.

3. The method of claim 1, wherein
the VH comprises any one of SEQ ID NOs: 1 to 9; and
the VL comprises any one of SEQ ID NOs: 11 to 15.

4. The method of claim 3, wherein the VH and the VL comprise:
SEQ ID NOs: 1 and 11,
SEQ ID NOs: 2 and 12,
SEQ ID NOs: 3 and 12,
SEQ ID NOs: 4 and 12,
SEQ ID NOs: 7 and 12,
SEQ ID NOs: 5 and 13,
SEQ ID NOs: 5 and 15,
SEQ ID NOs: 6 and 13,
SEQ ID NOs: 6 and 14,
SEQ ID NOs: 5 and 14,
SEQ ID NOs: 8 and 14, or
SEQ ID NOs: 9 and 14,
respectively.

5. The method of claim 1, wherein the VH comprises SEQ ID NO: 1 and the VL comprises SEQ ID NO: 11, and wherein the antibody comprises a human IgG1 constant region.

6. The method of claim 1, wherein the antibody or antigen-binding fragment
a) binds to aggregated or oligomeric alpha-synuclein,
b) does not bind to monomeric alpha-synuclein, or
c) a) and b).

7. The method of claim 1, wherein the antibody or antigen-binding fragment is bispecific.

8. The method of claim 7, wherein the bispecific antibody or antigen-binding fragment comprises a portion that binds insulin-like growth factor 1 receptor (IGF1R).

9. The method of claim 8, wherein the IGF1R-binding portion comprises a VH and a VL, wherein
the VH comprises heavy chain CDR1-3 set forth in SEQ ID NOs: 51-53, respectively, and
the VL comprises light chain CDR1-3 set forth in SEQ ID NOs: 46-48, respectively.

10. The method of claim 8, wherein the IGF1R-binding portion comprises a VH and a VL comprising SEQ ID NOs: 50 and 45, respectively.

11. The method of claim 8, wherein the IGF1R-binding portion is an scFv.

12. The method of claim 11, wherein the scFv comprises SEQ ID NO: 54.

13. The method of claim 8, wherein the IGF1R-binding portion is fused to the C-terminus of one or both heavy chains of the antibody.

14. The method of claim 8, wherein
one heavy chain of the antibody comprises one or more knob mutations, and
the other heavy chain of the antibody comprises one or more hole mutations.

15. The method of claim 14, wherein
a) the one or more knob mutations comprise T366W (Eu numbering);
b) the one or more hole mutations comprise T366S, L368A, and Y407V (Eu numbering); or
c) a) and b).

16. The method of claim 14, wherein the IGF1R-binding portion is located at the C-terminus of the knob heavy chain or at the C-terminus of the hole heavy chain.

17. The method of claim 7, wherein the bispecific antibody comprises heavy chains with an M428L mutation (Eu numbering).

18. A method of treating an alpha-synucleinopathy in a human subject in need thereof, comprising administering a bispecific antibody or antigen-binding fragment thereof that binds to human alpha-synuclein and IGF1R, wherein the antibody comprises a heavy chain comprising SEQ ID NO: 57 and a heavy chain comprising SEQ ID NO: 58; and two light chains each comprising SEQ ID NO: 59.

19. The method of claim 1, wherein the alpha-synucleinopathy is Parkinson's disease, dementia with Lewy bodies, multiple system atrophy, or Alzheimer's disease with amygdala Lewy bodies.

20. The method of claim 18, wherein the alpha-synucleinopathy is Parkinson's disease, dementia with Lewy bodies, multiple system atrophy, or Alzheimer's disease with amygdala Lewy bodies.
